(19) United States
(12) Patent Application Publication
(10) Pub. No.: US 2022/0184091 A1
(43) Pub. Date: Jun. 16, 2022

(54) Methods of Treating Cancer with Chk1 Inhibitors

(71) Applicants: Sierra Oncology, Inc., San Mateo, CA (US); The University of Texas M.D. Anderson Cancer Center, Houston, TX (US)

(72) Inventors: Christian Andrew Hassig, San Diego, CA (US); Snezana Milutinovic, San Diego, CA (US); Ryan James Hansen, San Diego, CA (US); Bryan William Strouse, Ann Arbor, MI (US); Lauren Byers, Houston, TX (US); Triparna Sen, Houston, TX (US)

(73) Assignees: Sierra Oncology, Inc., San Mateo, CA (US); The University of Texas M.D. Anderson Cancer Center, Houston, TX (US)

(21) Appl. No.: 17/441,725

(22) PCT Filed: Mar. 26, 2020
(86) PCT No.: PCT/US2020/025018

§ 371 Date: Sep. 22, 2021

Related U.S. Application Data
(60) Provisional application No. 62/825,275, filed on Mar. 28, 2019.

(51) Int. Cl. A61K 31/5377; A61P 35/00; A61K 31/7068; A61P 35/04; A61K 38/17

(52) U.S. Cl. CPC A61K 31/5377; A61P 35/00; A61K 38/177; A61P 35/04; A61K 31/7068

(57) ABSTRACT

Disclosed herein are methods of treatment by administering SRA737 as a monotherapy or in a combination therapy that is useful for treating patients with cancer.

### CROSS-REFERENCE TO RELATED APPLICATIONS

This application claims priority to U.S. Provisional Application No. 62/825,275, filed on Mar. 28, 2019, which is hereby incorporated by reference in its entirety.

### BACKGROUND OF THE INVENTION

Cells activate a signal transduction pathway when DNA is damaged. Signals activate the cell-cycle machinery to induce DNA repair and/or cell death to mitigate propagation. Checkpoint kinase 1 (Chk1) is an important bridge in cells when sensing DNA damage. See Cancer Biology & Therapy (2004) 3:3, 305-313, incorporated herein by reference. Chk1 plays a role in regulating numerous and wide-ranging cellular functions including: immune and inflammation responses, spindle formation, DNA damage signal transduction and generally, cellular apoptosis. Chk1 inhibitors abrogate DNA damage-induced cell cycle arrest in S and/or G 2/M phases. Currently, there are no Chk1 inhibitors that are approved therapies for inhibition of tumor growth. One Chk1 inhibitor in development is SRA737. SRA737 is described in international patent application number PCT/GB2013/051233, the disclosure of which is incorporated herein by reference in its entirety.

Gemcitabine-based chemotherapy has been used for treatment of pancreatic cancer, non-small cell lung cancer (NSCLC), breast cancer, ovarian cancer, and relapsed SCLC patients, but by itself has activity only in a minority of patients. Standard chemotherapies, including gemcitabine, and DNA damage repair (DDR) inhibitors may have overlapping toxicities which would limit combining these drugs at maximum doses in the clinic. Thus, low-dose gemcitabine (LDG) is currently being tested in combination with the Chk1 inhibitor, SRA737, in Phase I/II trials for multiple cancer types (NCT02797977).

### SUMMARY OF THE INVENTION

Disclosed herein is a method of treating a cancer, comprising administering to a subject with the cancer an effective amount of a SRA737 compound, wherein the effective amount is less than 2000 mg/day.

In some embodiments, the SRA737 compound is administered orally.

In some embodiments, wherein the SRA737 compound is administered daily. In some embodiments, the SRA737 compound is administered for at least 28 consecutive days. In some embodiments, the SRA737 compound is administered for at least 7 consecutive days.

In some embodiments, the SRA737 compound is administered intermittently. In some embodiments, the SRA737 compound is administered with at least ten (10) minutes, fifteen (15) minutes, twenty (20) minutes, thirty (30) minutes, forty (40) minutes, sixty (60) minutes, two (2) hours, three (3) hour, four (4) hours, six (6) hours, eight (8) hours, ten (10) hours, twelve (12) hours, fourteen (14) hours, eighteen (18) hours, twenty-four (24) hours, thirty-six (36) hours, forty-eight (48) hours, three (3) days, four (4) days, five (5) days, six (6) days, seven (7) days, eight (8) days, nine (9) days, ten (10) days, eleven (11) days, twelve (12) days, thirteen (13) days, fourteen (14) days, three (3) weeks, or four (4) weeks, delay between administrations.

In some embodiments, the SRA737 compound is administered over one or more 28 day cycles. In some embodiments, the SRA737 compound is administered on one or more days of the one or more 28 day cycles. In some embodiments, is administered on days 2, 3, 9, 10, 16, and 17 of the one or more 28 day cycles. In some embodiments, the method further comprises administering an initial dose of the SRA737 compound prior to the first of the one or more 28 day cycles. In some embodiments, the initial dose is administered 4 days, 5 days, 6 days, or 7 days prior to the first cycle of the one or more 28 day cycles. In some embodiments, the one or more 28 day cycles comprises 2, 3, 4, 5, 6 or more 28 day cycles.

In some embodiments, the SRA737 compound is administered following a dosing schedule selected from the group consisting of 5 days of dosing followed by 2 days of non-dosing each week, 1 week of daily dosing followed by 1, 2, or 3 weeks of non-dosing, 2 or 3 weeks of daily dosing followed by 1, or 2 weeks of non-dosing, and dosing on days 2 and 3 of a weekly cycle.

In some embodiments, the effective amount is administered in a single dose once a day. In some embodiments, half of the effective amount is administered twice a day.

In some embodiments, the effective amount is less than 1500 mg/day. In some embodiments, the effective amount is less than 1300 mg/day. In some embodiments, the effective amount is 1000 mg/day or less. In some embodiments, the effective amount is 900 mg/day or less. In some embodiments, the effective amount is 800 mg/day or less. In some embodiments, the effective amount is 700 mg/day or less. In some embodiments, the effective amount is 600 mg/day or less. In some embodiments, the effective amount is 500 mg/day or less. In some embodiments, the effective amount is 400 mg/day or less. In some embodiments, the effective amount is between 600 mg/day and 1300 mg/day. In some embodiments, the effective amount is between 300 mg/day and 1300 mg/day. In some embodiments, the effective amount is between 300 mg/day and 1000 mg/day. In some embodiments, the effective amount is between 300 mg/day and 800 mg/day. In some embodiments, the effective amount is between 500 mg/day and 1300 mg/day. In some embodiments, the effective amount is between 500 mg/day and 1000 mg/day. In some embodiments, the effective amount is between 500 mg/day and 800 mg/day. In some embodiments, the effective amount is selected from the group consisting of: 600 mg/day, 700 mg/day, 800 mg/day, 900 mg/day, 1000 mg/day, 1100 mg/day, and 1200 mg/day. In some embodiments, the effective amount is selected from the group consisting of: 40 mg/day, 80 mg/day, 300 mg/day, 500 mg/day, 600 mg/day, 700 mg/day, and 800 mg/day. In some embodiments, the effective amount is 300 mg/day. In some embodiments, the effective amount is 400 mg/day. In some embodiments, the effective amount is 500 mg/day. In some embodiments, the effective amount is 600 mg/day. In some embodiments, the effective amount is 700 mg/day. In some embodiments, the effective amount is 800 mg/day. In some embodiments, the effective amount is 900 mg/day. In some embodiments, the effective amount is 1000 mg/day.

In some embodiments, the cancer is metastatic cancer. In some embodiments, the cancer is a condition or disorder selected from the group consisting of: colorectal cancer, ovarian cancer, high grade serous ovarian cancer (HGSOC), non-small cell lung cancer (NSCLC), small cell lung cancer (SCLC), lung adenocarcinoma, prostate cancer, castration-resistant prostate cancer, bile duct cancer, cholangiocarcinoma, melanoma, uterine cancer, thyroid cancer, bladder cancer, breast cancer, cervical cancer, gastric cancer, endometrial cancer, hepatocellular cancer, leukemia, lymphoma, Non-Hodgkin's lymphoma, myeloma, brain cancer, neuroblastoma, squamous cell carcinoma, head and neck squamous cell carcinoma (HNSCC), and squamous cell carcinoma of the anus (SCCA), anogenital cancer, rectal cancer, pancreatic cancer, urothelial carcinoma, sarcoma and soft tissue sarcoma, metastatic colorectal cancer (CRC), platinum-resistant or intolerant HGSOC, advanced NSCLC, and metastatic castration-resistant prostate cancer (mCRPC), triple-negative breast cancer, invasive breast cancer, metastatic breast cancer, HER2 positive breast cancer and inflammatory breast cancer.

In some embodiments, the cancer is colorectal cancer. In some embodiments, the colorectal cancer is characterized as having a microsatellite instability or a deficiency in mismatch repair (MMR).

In some embodiments, the cancer is non-small cell lung cancer (NSCLC).

In some embodiments, the cancer is small cell lung cancer (SCLC).

In some embodiments, the cancer is HNSCC.

In some embodiments, the cancer is SCCA.

In some embodiments, the cancer is anogenital cancer.

In some embodiments, the cancer is prostate cancer. In some embodiments, the prostate cancer is metastatic castration-resistant prostate cancer (mCRPC).

In some embodiments, the cancer is ovarian cancer. In some embodiments, the ovarian cancer is high-grade serous ovarian cancer (HGSOC). In some embodiments, a tumor associated with the HGSOC is identified as having an increased expression of a Cyclin E1 (CCNE) gene. In some embodiments, the increased expression is a result of genetic amplification. In some embodiments, the tumor is identified as having somatic or germline BRCA1 and BRCA2 wild-type status.

In some embodiments, a tumor associated with the cancer is identified as having a gain of function mutation, amplification or overexpression of at least one oncogenic driver gene or other gene implicated in Chk1 pathway sensitivity. In some embodiments, the oncogenic driver gene is selected from the group consisting of MYC, MYCN, KRAS, and CCNE1.

In some embodiments, a tumor associated with the cancer is identified as having a loss of function or a deleterious mutation in at least one DNA damage repair (DDR) pathway gene implicated in Chk1 pathway sensitivity. In some embodiments, the DDR pathway gene is selected from the group consisting of ATM, CDK12, RAD51C. BRCA1, BRCA2, MRE11A, ATR, and an FA pathway gene. In some embodiments, the loss of function or the deleterious mutation is determined by establishing microsatellite instability or a deficiency in mismatch repair (MMR).

In some embodiments, a tumor associated with the cancer is identified as having a gain of function mutation or amplification of at least one replication stress gene implicated in Chk1 pathway sensitivity. In some embodiments, the replication stress gene is ATR or Chk1.

In some embodiments, a tumor associated with the cancer is identified as having a deleterious mutation in a tumor suppressor (TS) gene implicated in Chk1 pathway sensitivity. In some embodiments, a tumor associated with the cancer suppressor gene is selected from the group consisting of: RB1, TP53, ATM, RAD50, FBXW7 and PARK2.

In some embodiments, the subject is papillomavirus (HPV) positive.

In some embodiments, the subject is human.

In some embodiments, the method further comprises administering at least one or more additional effective amount of at least one or more further treatment, wherein the further treatment is selected from the group consisting of: a chemotherapeutic agent, an antibody or antibody fragment, a radiation treatment, an external inducer of replication stress, and a combination thereof.

In some embodiments, the further treatment is selected from the group consisting of: gemcitabine, olaparib, niraparib, rucaparib, talazoparib, cisplatin, a ribonucleotide reductase inhibitor, etoposide, SN-38/CPT-11, mitomycin C, and combinations thereof. In another embodiment, the further treatment is selected from the group consisting of ipilimumab, pembrolizumab, nivolumab, atezolumab, durvalumab, avelumab, epacadostat, indoximod, F-001287, B7-H1, and NLG919. In some embodiments, the further treatment comprises gemcitabine. In another embodiment, the further treatment comprises an immune checkpoint inhibitor. In some embodiments, the further treatment is administered daily. In some embodiments, the further treatment is administered on day 1 and the SRA737 compound is administered on days 2 and 3 of a weekly schedule. In some embodiments, the further treatment and the SRA737 compound are administered over one or more 28 day cycles. In some embodiments, the further treatment is administered on days 1, 8, and 15 of the one or more 28 day cycles, and the SRA737 compound is administered on days 2, 3, 9, 10, 16, and 17 of the one or more 28 day cycles. In some embodiments, the second effective amount of the further treatment is selected from the group consisting of: 50 mg/m²/day, 100 mg/m²/day, 150 mg/m²/day, 200 mg/m²/day, 250 mg/m²/day, and 300 mg/m²/day. In some embodiments, the second effective amount of the further treatment is 600 mg/m²/day or less. In some embodiments, the second effective amount of the further treatment is between 50 and 600 mg/m²/day. In some embodiments, the second effective amount of the further treatment is between 50 and 300 mg/m²/day. In some embodiments, the effective amount of the SRA737 compound is 80 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day. In some embodiments, the effective amount of the SRA737 compound is 150 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day. In some embodiments, the effective amount of the SRA737 compound is 300 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day. In some embodiments, the effective amount of the SRA737 compound is 500 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day. In some embodiments, the effective amount of the SRA737 compound is 600 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day. In some embodiments, the effective amount of the SRA737 compound is 700 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day. In some embodiments, the effective amount of the SRA737 compound is 800 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day. In some embodiments, the effective amount of the SRA737 compound is 900 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day. In some embodiments, the effective amount of the SRA737 compound is 1000 mg/day and the second effective amount of the further treatment is selected from the group consisting of: 50 mg/m²/day, 100 mg/m²/day, 150 mg/m²/day, 200 mg/m²/day, 250 mg/m²/day, and 300 mg/m²/day.

In another aspect, the method further comprises administering an effective amount of a third treatment, wherein the third treatment is selected from the group consisting of: a chemotherapeutic agent, an antibody or antibody fragment such as an immune checkpoint inhibitor, a radiation treatment, an external inducer of replication stress, and a combination thereof.

In some embodiments, the immune checkpoint inhibitor is selected from the group consisting of ipilimumab, pembrolizumab, nivolumab, atezolumab, durvalumab, avelumab, epacadostat, indoximod, F-001287, B7-H1, and NLG919. In some embodiments, the immune checkpoint inhibitor comprises an anti-PD-L1 agent. In some embodiments, the immune checkpoint inhibitor comprises an anti-PD1 agent. In some embodiments, the third treatment is administered daily. In some embodiments, the further treatment is administered on day 1 and the SRA737 compound is administered on days 2 and 3 of a weekly schedule. In some embodiments, the third treatment, the further treatment and the SRA737 compound are administered over one or more 28 day cycles. In some embodiments, the third treatment is administered on days 1, 8, and 15 of the one or more 28 day cycles, and the SRA737 compound is administered on days 2, 3, 9, 10, 16, and 17 of the one or more 28 day cycles.

In some embodiments, more than one therapeutic agent may be administered to a subject. For example, both SRA737 and low-dose gemcitabine (LDG) may be administered. In another example, both SRA737 and an immune checkpoint inhibitor may be administered. In another example, both LDG and an immune checkpoint inhibitor may be administered.

In some embodiments, more than two therapeutic agents may be administered to a subject. For example, SRA737, LDG, and an immune checkpoint inhibitor may be administered.

In some embodiments, more than one therapeutic agent may be administered within a set period of time. For example, more than one therapeutic agent may be administered within one day, two days, three days, four days, five days, six days, seven days, one week, two weeks or more.

In some embodiments, two or more therapeutic agents (e.g., SRA737, LDG, and an immune checkpoint inhibitor) may be administered within a set period of time. In another embodiment, the one or more therapeutic agents may be administered at the same times. In yet another embodiment, the one or more therapeutic agents may be administered at different times. For example, a first therapeutic agent may be administered at a first time and a second therapeutic agent may be administered at a second time, which the second time differs from the first time (e.g., by minutes, hours, or days). For instance, a first therapeutic agent (e.g., LDG) may be administered at a first time and a second agent (e.g., SRA737) may be administered at a second time, which the second time is at least one hour later than the first time. In another embodiment, the second therapeutic agent may be administered at least one hour, two hours, four hours, six hours, eight hours, ten hours, twelve hours, 20 hours, 24 hours, 48 hours, 72 hours, or 96 hours after the first therapeutic agent is administered. Similarly, a third therapeutic agent (e.g., an immune checkpoint inhibitor) may be administered at a third time, which third time differs from the first time. For example, the third therapeutic agent may be administered at the same time as the second therapeutic agent. Alternatively, the third therapeutic agent may be administered after the second therapeutic agent, such as at least one hour after the second therapeutic agent is administered. For example, the third therapeutic agent may be administered at least one hour, two hours, four hours, six hours, eight hours, ten hours, twelve hours, 20 hours, 24 hours, 48 hours, 72 hours, or 96 hours after the second therapeutic agent is administered. Alternatively or in addition to, the third therapeutic agent may be administered at least one hour, two hours, four hours, six hours, eight hours, ten hours, twelve hours, 20 hours, 24 hours, 48 hours, 72 hours, or 96 hours after the first therapeutic agent is administered.

In some embodiments, a second therapeutic agent (e.g., SRA737) is administered at least two hours after a first therapeutic agent (e.g., LDG) is administered, and a third therapeutic agent (e.g. an immune checkpoint inhibitor) is administered at least one hour after the second therapeutic agent is administered. For example, a second therapeutic agent (e.g., B) may be administered between 12-24 hours after a first therapeutic agent (e.g., A) is administered, and a third therapeutic agent (e.g., C) may be administered between 12-24 hours after the second therapeutic agent is administered.

Where more than one therapeutic agent is administered to the subject, the more than one therapeutic agents may be administered at the same or different doses (e.g., as described herein). For example, the first therapeutic agent may be administered at a first dose and a second therapeutic agent may be administered at a second dose, which second dose differs from the first dose. Similarly, the more than one therapeutic agents may be administered by the same or different routes. For example, the first therapeutic agent may be administered by a first administration route (e.g., via injection) and the second therapeutic agent may be administered by a second administration route that differs from the first administration route (e.g., orally).

Administration of multiple different therapeutic agents within a set period of time may constitute a treatment cycle. For example, a treatment cycle may comprise administration of first, second, and third therapeutic agents (e.g., A, B, and C) within a set period of time (e.g., within one day, two days, three days, four days, five days, six days, seven days, or more). A subject may undergo multiple treatment cycles. For example, a subject may undergo two or more treatment cycles, such as three, four, five, or more treatment cycles. In some cases, each treatment cycle may be approximately the same (e.g., the same therapeutic agents may be administered within approximately the same times). In other cases, one or more treatment cycles may differ from one another. For example, a first treatment cycle may comprise administration of first, second, and third therapeutic agents at first, second, and third times (e.g., as described herein), and a second treatment cycle may comprise administration of the first, second, and third therapeutic agents at fourth, fifth, and sixth times, where at least one of the fourth, fifth, and sixth times differs from the first, second, and third times, respectively. In another example, a first treatment cycle may comprise administration of first, second, and third therapeutic agents at first, second, and third times (e.g., as described herein), and a second treatment cycle may comprise administration of only the first and second therapeutic agents. In some cases, a subject may undergo two or more treatment cycles within a set period of time, such as within one month, two months, three months, four months, five months, six months, or more. In some cases, a subject may undergo evaluation (e.g., as described herein) after a treatment cycle to determine whether an additional treatment cycle should be administered, and/or whether an additional treatment cycle should be modified relative to the previous treatment cycle (e.g., by changing a dose of one or more therapeutic agents, the spacing between administration of one or more therapeutic agents, and/or by adding a therapeutic agent to or removing a therapeutic agent from the treatment cycle).

In some embodiments, the cancer is urothelial carcinoma. In some embodiments, the urothelial carcinoma is selected from the group consisting of: (a) unresectable urothelial carcinomas of the bladder, upper urinary tract, or urethra, and (b) metastatic urothelial carcinomas of the bladder, upper urinary tract, or urethra. In some embodiments, the cancer is HGSOC. In some embodiments, a tumor associated with the HGSOC is identified as having somatic or germline BRCA1 and BRCA2 wild-type status. In some embodiments, the cancer is small cell lung cancer. In some embodiments, the cancer is soft tissue sarcoma. In some embodiments, the soft tissue sarcoma is selected from the group consisting of: undifferentiated pleiomorphic sarcoma, malignant fibrous histiocytoma (MFH)/high-grade spindle cell sarcoma, pleomorphic liposarcomas, leiomyosarcoma, and dedifferentiated liposarcoma. In some embodiments, the cancer is cervical or anogenital cancer. In some embodiments, the cervical or anogenital cancer is selected from the group consisting of: advanced/metastatic squamous cell carcinoma of the anus, penis, vagina, and vulva.

In some embodiments, the method results in growth inhibition of a tumor associated with the cancer. In some embodiments, the growth inhibition of the tumor associated with the cancer is a minimum growth inhibition of at least 10%, at least 20%, at least 30%, at least 40%, at least 50%, at least 60%, at least 70%, at least 80%, or at least 90% relative to an untreated tumor. In some embodiments, the method results in a regression of a tumor associated with the cancer relative to a baseline measurement. In some embodiments, the regression is a 30% regression of the tumor associated with the cancer relative to the baseline measurement. In some embodiments, the regression is a complete regression of the tumor associated with the cancer relative to the baseline measurement.

In some embodiments, the method results in cytotoxicity of a tumor associated with the cancer.

In some embodiments, the method results in a partial response, a complete response, or a stable disease in the subject relative to a baseline measurement. In some embodiments, the method results in a partial response in the subject relative to a baseline measurement. In some embodiments, the method results in a complete response in the subject relative to a baseline measurement. In some embodiments, the method results in a stable disease in the subject relative to a baseline measurement.

In some embodiments, the method results in a plasma Cₘᵢₙ of at least 100 ng/ml of the SRA737 compound for at least 24 hours in the subject after administration. In some embodiments, the method results in a plasma Cₘᵢₙ of at least 100 nM of the SRA737 compound for at least 24 hours in the subject after administration.

In some embodiments, the method results in a plasma AUC₀₋₂₄ of at least 100 ng·h/mL, at least 300 ng·h/mL, at least 600 ng·h/mL, at least 800 ng·h/mL, at least 1000 ng·h/mL, at least 1600 ng·h/mL, at least 2300 ng·h/mL, at least 2500 ng·h/mL, at least 3000 ng·h/mL, at least 3500 ng·h/mL, at least 8000 ng·h/mL, at least 12000 ng·h/mL, at least 15000 ng·h/mL, at least 18000 ng·h/mL, at least 20000 ng·h/mL, at least 25000 ng·h/mL, or at least 29000 ng·h/mL of the SRA737 compound in the subject after administration. In some embodiments, the method results in a plasma AUC₀₋₁₂ of at least 400 ng·h/mL, at least 500 ng·h/mL, at least 600 ng·h/mL, at least 1600 ng·h/mL, at least 2600 ng·h/mL, at least 4500 ng·h/mL, at least 5000 ng·h/mL, at least 8000 ng·h/mL, at least 8000 ng·h/mL, at least 1000 ng·h/mL of the SRA737 compound in the subject after administration.

In some embodiments, the method results in a plasma Cₘₐₓ of at least 500 ng/mL, at least 600 ng/mL, at least 800 ng/mL, at least 100 ng/mL, at least 150 ng/mL, at least 175 ng/mL, at least 350 ng/mL, at least 990 ng/mL, at least 1980 ng/mL, at least 2000 ng/mL, or at least 3228 ng/mL of the SRA737 compound in the subject after administration. In some embodiments, wherein the method results in a plasma Cₘₐₓ of less than 500 ng/mL, less than 600 ng/mL, less than 800 ng/mL, less than 100 ng/mL, less than 150 ng/mL, less than 175 ng/mL, less than 350 ng/mL, less than 990 ng/mL, less than 1980 ng/mL, less than 2000 ng/mL, or less than 3228 ng/mL of the SRA737 compound in the subject after administration. In some embodiments, the method results in a plasma Cₘₐₓ between 500 and 3200 ng/mL of the SRA737 compound in the subject after administration. In some embodiments, the method results in a plasma Cₘₐₓ between 500 and 2400 ng/mL of the SRA737 compound in the subject after administration. In some embodiments, the method results in a plasma Cₘₐₓ between 500 and 650 ng/mL of the SRA737 compound in the subject after administration. In some embodiments, the method results in a plasma Cₘₐₓ between 500 and 550 ng/mL of the SRA737 compound in the subject after administration. In some embodiments, the method results in a plasma Cₘₐₓ between 500 and 5500 ng/mL of the SRA737 compound in the subject after administration. In some embodiments, the method results in a plasma Cₘₐₓ between 500 and 4000 ng/mL of the SRA737 compound in the subject after administration.

In some embodiments, the subject has fasted prior to administering the effective amount of the SRA737 compound. In some embodiments, the subject has fasted 30 minutes or more, 1 hour or more, 2 hours or more, 3 hours or more, or 4 hours or more fasted prior to administering the effective amount of the SRA737 compound. In some embodiments, has fasted 2 hours or more fasted prior to administering the effective amount of the SRA737 compound.

In some embodiments, the method further comprises the subject fasting following administering the effective amount of the SRA737 compound. In some embodiments, the subject fasts 30 minutes or more, 1 hour or more, 2 hours or more, 3 hours or more, or 4 hours or more fasted following administering the effective amount of the SRA737 compound. In some embodiments, the subject fasts 1 hours or more following administering the effective amount of the SRA737 compound.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 schematizes basic mechanisms of DNA damage response (DDR) therapeutics.

FIG. 2 illustrates the activation of the STING pathway or the innate immune system by DNA damage in SCLC models.

FIG. 3 shows the effect of SRA737 on gemcitabine-induced Chk1 S296 autophosphorylation in HT29 human tumor xenografts in mice as measured by western blotting.

FIG. 4 shows dose proportional concentrations of SRA737 in plasma and tumor in a HT29 xenograft model. For each dose, columns from left to right are concentrations in plasma at 6 hours, plasma at 24 hours, tumor at 6 hours, and tumor at 24 hours, respectively.

FIG. 5 illustrates clinical trial dosing regimens used to establish pharmacokinetics and safety of SRA737 combination therapy with gemcitabine.

FIG. 6 shows IC50 values of treatment with SRA737 in SCLC, NSCLC, pancreatic (SW1990), colon (SNUC1) and bladder (5637) cell lines.

FIG. 7 shows the formation of micronuclei in two cells lines, H69 and EMT-6, treated with 1 μM SRA737 for 24 hours or 48 hours

FIG. 8 shows the quantification of micronuclei-containing cells in multiple cell lines, including two human SCLC cell lines, H69 and H446, and one SCLC murine cell line, RPP; two NSCLC cells lines, Calu-1 and Calu-6; and one breast cancer cell line, EMT6/P, after treatment with SRA737 (1 μM and 5 μM respectively) for 24 hours. Data presented as mean±SD and p values by t-test ***p<0.001.

FIG. 9 shows Western blots of PD-L1 levels in SCLC cell lines treated with control or with SRA737 at 1 μM.

FIG. 10 shows the surface PD-L1 expression in SCLC cell lines as measured by flow cytometry, following SRA737 treatment for 72 hours. Data presented as mean±SD and p values by t-test ***p<0.001.

FIG. 11 shows Western blot analysis of protein markers in the STING pathway, including total and phospho STING (S366), total and phospho IRF3 (S396), in lysates collected from two SCLC cell lines treated with SRA737 (1 μM) for 24 and 72 hours respectively. “Con” indicates control.

FIG. 12 shows the quantitative polymerase chain reaction (qPCR) measurement of CCL5, CXCL10 and IFNβ mRNA expression in three SCLC cell lines treated with SRA737 for 24 and 72 hours respectively. “Con” indicates control. Data presented as mean±SD and p values by t-test ***p<0.001.

FIG. 13 shows the cell viability curves in response to SRA737 for four SCLC cell lines, namely DMS273, H128, H69, and H719. The corresponding IC50 values of SRA737 in these cell lines are illustrated in FIG. 6.

FIGS. 14A-B show the comparative analysis of protein expression between sensitive and resistant cells to SRA737, demonstrating that cMYC protein is a marker for SRA737 sensitivity, and Bcl2, cKit, and E-cadherin are markers of resistance to SRA737. Protein expression was measured by Reverse Phase Protein Array (RPPA) and only proteins with a greater than 2-fold change and p<0.05 are shown.

FIG. 15A shows tumor volume of mice with B6129F1 immunocompetent flank RPP tumor that were treated with SRA737 (100 mg/kg, 3-days-on/7-days-off or 5-days-on/7-days-off) and/or anti-PD-L1 (300 μg, 1-day-on/7-days-off) for up to 36 days.

FIG. 15B shows a survival plot with Kaplan-Meyer survival curves and p-values by Mantel-Cox test * p<0.05, **p<0.01, ***p<0.001.

FIG. 16 shows the immunoblot analysis in a cohort of tumors from each treatment group, resected at Day 21, for PD-L1 and STING pathway proteins pSTING_S366, STING, pIRF3_S396, and IRF3.

FIG. 17 shows the quantitative PCR (qPCR) measurement of IFNβ, CXCL10, and CCL5 mRNA expression in SCLC tumors resected after 21 days of treatment with SRA737 5/7 days in RPP flank model. Data presented as mean±SD and p values by t-test * p<0.05, **p<0.01, ***p<0.001.

FIG. 18 shows the tumor volumes of mice that were injected with HT29 human colon cancer cells and were treated with LDG (40 mg/kg, 1-day-on/7-days-off) plus SRA737 (100 mg/kg, 2-days-on/7-days-off or 5-days-on/7-days-off). Tumor volume changes (means+/−standard error of the mean [SEM; error bars]) are shown. P values represented as * p<0.05, **p<0.01, ***p<0.001.

FIG. 19 shows survival rate of H209, H524, and RPP cancer cells after treatments with gemcitabine.

FIGS. 20A-B illustrate the T-cell (FIG. 20A) and APC panel (FIG. 20B) markers used for FACS analyses to identify infiltrating immune sub-populations.

FIGS. 21A-D show the immune infiltrate profiles of SCLC tumors, growth of which is graphed in FIG. 25, harvested at Day 21. The immune profiles were analyzed by FACS at the endpoint with representative plots and cumulative data for all tumors shown. FACS analyses of CD3+ total T-cells, CD4+ helper T-cells, CD25+FOXP3+T-regulatory cells, and F4/80− CD11b+CD11c− are shown in FIGS. 21A-D respectively. The statistical summary is shown with ANOVA test. *, p<0.05; **, p<0.01; ***, p<0.001.

FIGS. 22A-C show the tumor volumes in immunocompetent flank tumor bearing mice that were injected with bladder cancer cells MBT-2 (FIG. 22A), colon cancer cells MC38 (FIG. 22B), and pancreatic cancer cells Pan02 (FIG. 22C) after being treated with SRA737 monotherapy (100 mg/kg, 5/7 days), anti-PD1 monotherapy twice weekly (10 mg/kg), or SRA737 and anti-PD1 combination therapy for up to 28 days. Tumor volumes presented as means+/−SEM. * p<0.05, **p<0.01, ***p<0.001.

FIGS. 23A-D show the qPCR measurement of CXCL10 and CCL5 mRNA expression in MBT-2 (FIG. 23A and FIG. 23B) and MC38 (FIG. 23C and FIG. 23D) tumors resected after 7 or 14 days of treatment with SRA737 5/7 days. Data presented as mean±SD and p values by t-test * p<0.05, **p<0.01, ***p<0.001.

FIG. 24 shows the quantification of CD4 and CD8 IHC staining in tumors resected from MC38 colon models after 14 days of treatment of SRA737 (7 days on; 7 days off) with or without anti-PD-1 (Days 3, 7 and 11). Measurements were taken post treatment on day 15.

FIG. 25A shows the tumor volumes of RPP tumor-bearing B6129F1 immunocompetent mice that were treated with anti-PD-L1 monotherapy at 300 μg (1-day-on/6-days-off), LDG monotherapy at 40 mg/kg (1-day-on/6-days-off), SRA737 monotherapy at 100 mg/kg (2-days-on/5-days-off), and their double and triple therapy combinations. Tumor volume presented as mean+/−SEM, * p<0.05, **p<0.01, ***p<0.001.

FIG. 25B shows the treatment schedule of the triple combination therapy comprising LDG, SRA737, and anti-PD-L1.

FIGS. 26A-B show the immune infiltration profiles of SCLC tumors harvested at Day 21 with fluorescence-activated cell sorting (FACS) analyses for CD3⁺CD45⁺CD8⁺ cytotoxic T-cells (FIG. 26A) and PD1⁺TIM3⁺exhausted T-cells (FIG. 26B).

FIGS. 27A-D show the immune infiltration profiles of SCLC tumors harvested at Day 21 with FACS analyses for CD86⁺ CD68⁺iNOS⁺ M1 macrophage (FIG. 27A), Arg1⁺CD68⁺M2 macrophage (FIG. 27B), F4/80⁻ MHCII⁺ GR1⁻ Dendritic Cells (FIG. 27C) and myeloid-derived suppressor cells or MDSCs (FIG. 27D). p-values by ANOVA, ns, no significance; *, p<0.05; **, p<0.01; ***, p<0.001.

FIG. 28 shows the qPCR measurement of IFNβ, CXCL10 and CCL5 mRNA expression in RPP tumors resected after 21 days of treatments of monotherapies of SRA737, LDG, and anti-PD-L1, and various double and triple combinations thereof. Data presented as mean±SD and p values by t-test * p<0.05, **p<0.01, ***p<0.001.

FIGS. 29A-D shows the immune infiltration profiles of SCLC tumors harvested at Day 21 with FACS analyses for CD3⁺ total T-cells (FIG. 29A), CD4⁺ helper T-cells (FIG. 29B), CD25⁺FOXP3⁺ T-regulatory cells (FIG. 29C), and C4/80− CD11b⁺ CD11c⁻ granulocytes (FIG. 29D). p-values by ANOVA, ns, no significance; *, p<0.05; **, p<0.01; ***, p<0.001.

### DETAILED DESCRIPTION OF THE INVENTION

Disclosed herein are methods of inhibiting tumor growth in a subject, e.g., a human, by administration of an effective amount of the Chk1 inhibitor SRA737. Also disclosed herein are methods of inhibiting tumor growth in a subject, e.g., a human, by administration of an effective amount of the Chk1 inhibitor SRA737 in a combination therapy.

### Definitions

Unless otherwise defined herein, all technical and scientific terms used herein have the meaning commonly understood by a person skilled in the art to which this invention pertains.

The practice of the present invention includes the use of conventional techniques of organic chemistry, molecular biology (including recombinant techniques), microbiology, cell biology, biochemistry and immunology, which are within the skill of the art.

In this application, reference will be made to a number of technical designations. All numerical designations, e.g., pH, temperature, time, concentration, and weight, including ranges of each thereof, are approximations that typically may be varied (+) or (−) by increments of 0.1, 1.0, or 10.0, as appropriate. All numerical designations may be understood as preceded by the term “about.” Reagents described herein are exemplary and equivalents of such may be known in the art.

Compounds utilized in the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. The compounds of the present invention may also contain unnatural proportions of atomic isotopes at one or more of the atoms that constitute such compounds. For example, the compounds may be radiolabeled with radioactive isotopes, such as for example, and without limitation, tritium (³H), iodine-125 (¹²¹I), or carbon-14 (¹⁴C). All isotopic variations of the compounds of the present invention, whether radioactive or not, are intended to be encompassed within the scope of the present invention.

The term “subject” refers to any mammal including humans, and mammals such as those animals of veterinary and research interest that are including, but not limited to: simians, cattle, horses, dogs, cats, and rodents.

The term “administering” or “administration of”” a drug and/or therapy to a subject (and grammatical equivalents of this phrase) refers to both direct or indirect administration, which may be administration to a subject by a medical professional, may be self-administration, and/or indirect administration, which may be the act of prescribing or inducing one to prescribe a drug and/or therapy to a subject.

The term “coadministration” refers to two or more compounds administered in a manner to exert their pharmacological effect during the same period of time. Such coadministration can be achieved by either simultaneous, contemporaneous, or sequential administration of the two or more compounds.

The term “treating” or “treatment of” a disorder or disease refers to taking steps to alleviate the symptoms of the disorder or disease, e.g., tumor growth or cancer, or otherwise obtain some beneficial or desired results for a subject, including clinical results. Any beneficial or desired clinical results may include, but are not limited to, alleviation or amelioration of one or more symptoms of cancer or conditional survival and reduction of tumor load or tumor volume; diminishment of the extent of the disease; delay or slowing of the tumor progression or disease progression; amelioration, palliation, or stabilization of the tumor and/or the disease state; or other beneficial results.

The term “in situ” or “in vitro” refers to processes that occur in a living cell growing separate from a living organism, e.g., growing in tissue culture.

The term “in vivo” refers to processes that occur in a living organism.

The term “Chk1” or “CHEK1” or “checkpoint kinase 1” refers to serine/threonine-protein kinase that is encoded by the CHEK1 gene.

The term “effective amount” means an amount sufficient to produce a desired effect, e.g., an amount sufficient to inhibit tumor growth.

The term “reduction” of a symptom or symptoms (and grammatical equivalents of this phrase) refers to decreasing the severity or frequency of the symptom(s), or elimination of the symptom(s).

It must be noted that, as used in the specification and the appended claims, the singular forms “a,” “an” and “the” include plural referents unless the context clearly dictates otherwise.

Methods of the Invention

Disclosed herein are methods of inhibiting tumor growth in a subject, e.g., a human, by administration of the Chk1 inhibitor SRA737. A detailed description of the compounds, kits comprising the compounds, and methods of use thereof are found below.

Tumor Inhibition

The present disclosure is directed to methods using an effective amount of the compound SRA737 to inhibit the progression of, reduce the size in aggregation of, reduce the volume of, and/or otherwise inhibit the growth of a tumor. Also provided herein are methods of treating the underlying disease, e.g., cancer, and extending the survival of the subject.

In some aspects, provided for is a method of inhibiting the growth of a tumor in a subject in need thereof, the method comprising administering to the subject an effective amount of SRA737. In some aspects, the disclosure provides for a method of administering to the subject an effective amount of SRA737 to inhibit growth of a tumor, wherein tumor growth is reduced by 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% as measured by tumor volume. In some aspects, the disclosure provides for a method of administering to the subject an effective amount of SRA737 to inhibit growth of a tumor, wherein tumor growth is reduced by 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% as measured by the absolute size of the tumor. In some aspects, the disclosure provides for a method of administering to the subject an effective amount of SRA737 to inhibit the growth of a tumor, wherein tumor growth is reduced by 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% as measured by the expression levels of tumor markers for that type of tumor.

In some aspects, provided for is a method of treating a cancer, comprising administering to a subject with the cancer an effective amount of a SRA737 compound. In some aspects, provided for is a method of treating a cancer, comprising administering to a subject with the cancer an effective amount of a SRA737 compound, wherein the method results in a regression of a tumor. The regression, in general, is determined relative to a baseline measurement. The regression can be a partial regression or a complete regression. The regression can, in general, be measured by any assay useful for quantitating size, volume, and/or growth of a tumor, e.g., medical imaging techniques known in the art. The regression can be a 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% regression as measured by tumor volume. The regression can be a 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% regression as measured by the absolute size of the tumor. The regression can be a 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% regression as measured by the expression levels of tumor markers for that type of tumor. The regression can be a 30% regression. The regression can be a 30% regression as measured by any assay useful for quantitating size, volume, and/or growth of a tumor, e.g., medical imaging techniques known in the art.

The present disclosure is also directed to methods using an effective amount of the compound SRA737 and a second effective amount of a further treatment to inhibit the progression of, reduce the size in aggregation of, reduce the volume of, and/or otherwise inhibit the growth of a tumor. Also provided herein are methods of treating the underlying disease, e.g., cancer, and extending the survival of the subject. In some aspects, provided for is a method of inhibiting the growth of a tumor in a subject in need thereof, the method comprising administering to the subject an effective amount of SRA737 and a second effective amount of a further treatment. In some aspects, the disclosure provides for a method of administering to the subject an effective amount of SRA737 and a second effective amount of a further treatment to inhibit growth of a tumor, wherein tumor growth is reduced by 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% as measured by tumor volume. In some aspects, the disclosure provides for a method of administering to the subject an effective amount of SRA737 and a second effective amount of a further treatment to inhibit growth of a tumor, wherein tumor growth is reduced by 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% as measured by the absolute size of the tumor. In some aspects, the disclosure provides for a method of administering to the subject an effective amount of SRA737 and a second effective amount of a further treatment to inhibit growth of a tumor, wherein tumor growth is reduced by 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% as measured by the expression levels of tumor markers for that type of tumor.

In some aspects, provided for is a method of treating a cancer, comprising administering to a subject with the cancer an effective amount of a SRA737 compound and a second effective amount of a further treatment. In some aspects, provided for is a method of treating a cancer, comprising administering to a subject with the cancer an effective amount of a SRA737 compound and a second effective amount of a further treatment, wherein the method results in a regression of a tumor. The regression, in general, is determined relative to a baseline measurement. The regression can be a partial regression or a complete regression. The regression can, in general, be measured by any assay useful for quantitating size, volume, and/or growth of a tumor, e.g., medical imaging techniques known in the art. The regression can be a 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% regression as measured by tumor volume. The regression can be a 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% regression as measured by the absolute size of the tumor. The regression can be a 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% regression as measured by the expression levels of tumor markers for that type of tumor. The regression can be a 30% regression. The regression can be a 30% regression as measured by any assay useful for quantitating size, volume, and/or growth of a tumor, e.g., medical imaging techniques known in the art.

Additional Methods

The present disclosure also provides for a method of inhibiting Chk1 activity in a cell, the method comprising contacting the cell first with an effective amount of SRA737 and contacting the cell's environment with a second effective amount of an immune checkpoint inhibitor.

The present disclosure also provides for a method of activating innate immune signaling pathways in a cell, the method comprising contacting the cell with a first effective amount of SRA737 and contacting the cell's environment with a second effective amount of an immune checkpoint inhibitor. In one aspect, the activation of the innate signaling pathway synergizes with the immune checkpoint inhibitor to reduce growth of a tumor. In some aspects, contacting the cell with SRA737 activates innate immune signaling pathways and results in the release of cytokines, chemokines, or both cytokines and chemokines from the cell that function to activate an immune response. In another aspect, the release of cytokines, chemokines, or both cytokines and chemokines from the cell results in increased recruitment of immune cells to the cell's environment. In a further aspect, the immune checkpoint inhibitor targets the immune cells recruited to the cell's environment resulting in an increased or synergistic anti-tumor activity. In certain aspects, the innate immune signaling pathway is the Stimulator of Interferon Genes (STING) pathway. In certain aspects, the innate immune signaling pathway is an interferon signaling pathway. In particular aspects, additional compounds are administered that modulate the STING pathway and increase SRA737-mediated activation of innate immune signaling in a cell, such as additional activating ligands of the STING pathway, including, but not limited to, cyclic dinucleotides. In other aspects, SRA737 acts to modulate the expression of immune signaling cell surface markers (e.g. PDL-1, MHC I etc.) to enhance immune cell-mediated anti-tumor activity.

The present disclosure also provides for a method of increasing the incidence of neoantigens within a cell, the method comprising contacting the cell with a first effective amount of SRA737 and contacting the cell's environment with a second effective amount of an immune checkpoint inhibitor. In one aspect, the increased incidence of the neoantigens synergizes with the immune checkpoint inhibitor to reduce growth of a tumor. In some aspects, contacting the cell with SRA737 increases the mutational burden within the cell. In one aspect, the increased mutational burden results in the increased incidence of neoantigens. In a further aspect, the increased incidence of neoantigens results in an increased stimulation of the immune system. In an additional aspect, the increased stimulation of the immune system, due to the increased incidence of neoantigens, synergizes with the immune checkpoint inhibitor resulting in an increased or synergistic anti-tumor activity.

The present disclosure also provides for a method of inhibiting an immune checkpoint pathway, the method comprising contacting the cell with a first effective amount of SRA737 and contacting the cell's environment with a second effective amount of an immune checkpoint inhibitor. In some aspects, the disclosure provides for a method of inhibiting Chk1 activity in a cell or an immune checkpoint pathway, wherein the first effective amount and second effective amount is an amount that produces an IC₅₀ value of no more than 0.001 μM, no more than 0.01 μM, no more than 0.1 μM, no more than 1 μM, no more than 2 μM, no more than 3 μM, no more than 5 μM, no more than 6 μM, no more than 8 μM, no more than 10μM, no more than 12 μM, no more than 14 μM, no more than 16 μM, no more than 18 μM, no more than 20 μM, no more than 25 μM, no more than 30 μM, no more than 35 μM, no more than 40 μM, no more than 50 μM, no more than 75 μM, or no more than 100 μM.

Types of Tumors

In some aspects, the present disclosure provides for methods of inhibiting the growth of a tumor wherein the tumor is from a cancer that is colorectal cancer, ovarian cancer, high grade serous ovarian cancer (HGSOC), non-small cell lung cancer (NSCLC), small cell lung cancer (SCLC), lung adenocarcinoma, prostate cancer, castration-resistant prostate cancer, bile duct cancer, cholangiocarcinoma, melanoma, uterine cancer, thyroid cancer, bladder cancer, breast cancer, cervical cancer, gastric cancer, endometrial cancer, hepatocellular cancer, leukemia, lymphoma, Non-Hodgkin's lymphoma, myeloma, brain cancer, neuroblastoma, squamous cell carcinoma, head and neck squamous cell carcinoma (HNSCC), and squamous cell carcinoma of the anus (SCCA), anogenital cancer (e.g., anal cancer), rectal cancer, pancreatic cancer, urothelial carcinoma, sarcoma and soft tissue sarcoma, metastatic colorectal cancer (CRC), platinum-resistant or intolerant HGSOC, advanced NSCLC, and metastatic castration-resistant prostate cancer (mCRPC), triple-negative breast cancer, invasive breast cancer, metastatic breast cancer, HER2 positive breast cancer and inflammatory breast cancer.

Accordingly, the present disclosure also provides for methods of treating a cancer in a subject in need thereof, the method comprising administering an effective amount of SRA737 to the subject. In some aspects, methods are disclosed for the treatment of cancer wherein the cancer is colorectal cancer, ovarian cancer, high grade serous ovarian cancer (HGSOC), non-small cell lung cancer (NSCLC), small cell lung cancer, lung adenocarcinoma, prostate cancer, castration-resistant prostate cancer, bile duct cancer, cholangiocarcinoma, melanoma, uterine cancer, thyroid cancer, bladder cancer, breast cancer, cervical cancer, gastric cancer, endometrial cancer, hepatocellular cancer, leukemia, lymphoma, Non-Hodgkin's lymphoma, myeloma, brain cancer, neuroblastoma, squamous cell carcinoma, head and neck squamous cell carcinoma (HNSCC), and squamous cell carcinoma of the anus (SCCA), anogenital cancer (e.g., anal cancer), rectal cancer, pancreatic cancer, urothelial carcinoma, sarcoma and soft tissue sarcoma, metastatic colorectal cancer (CRC), platinum-resistant or intolerant HGSOC, advanced NSCLC, and metastatic castration-resistant prostate cancer (mCRPC), triple-negative breast cancer, invasive breast cancer, metastatic breast cancer, HER2 positive breast cancer and inflammatory breast cancer.

Clinical Endpoints

Provided herein are methods for inhibiting the growth of a tumor in a subject and/or cell, wherein the conditions of said methods are such that the method results in a clinically relevant endpoint.

Tumor growth occurs when one or more biological cells grow and divide much more rapidly resulting in an increase in the number of cells in comparison to the normal and healthy process of cells division. This phenomenon is an indication that the cells are in a disease state such as cancer or pre-cancer. Moreover, tumor growth oftentimes comes about in discrete stages prior to the agglomerated cells forming a tumor.

There are several methods the skilled artisan can use to measure cell replication rates. The overall metabolic activity inside a cell can be measured via a labeled biological product. For example, there are several commercially available dyes (e.g. MTT) that can penetrate the cell and interact with certain enzymes and other factors to produce a detectable product. Also, cellular biomarkers can be measured in a cell. For example, a BrdU assay can incorporate a thymidine derivative into cellular DNA and be detected with an antibody. Proliferating cell nuclear antigen (PCNA) is another such biomarker for detection. Besides tagging techniques, the skilled artisan can also use for example, microscopy or flow cytometry to allow for cell counts.

In one aspect, cellular replication is measured by a clinical endpoint that includes: a quality of life (QOL) score, duration of response (DOR, clinical benefit rate (CBR), patient reported outcomes (PRO), an objective response rate (ORR) score, a disease-free survival (DFS) or progression-free survival (PFS), a time to progression (TTP), an Overall Survival (OS), a time-to-treatment failure (TTF), RECIST criteria, and/or a Complete Response. the clinical endpoints can be determined using methods well known to one of skill in the art.

In some aspects, the present disclosure provides methods wherein the growth of the tumor is reduced no more than 5, 10, 20, 40, 50, 60, 80, 90, 95, 97, 99, or 99.9% after administration of the effective amount of SRA737.

In some aspects, the present disclosure provides methods wherein the % reduction is calculated based on measurement(s) of one or more clinical endpoints.

In some aspects, the present disclosure provides methods wherein the growth of the tumor is reduced as measured by an increase or a decrease in total cell count in a MTT assay, or by change in genetic profile as measured by a ctDNA assay, by no more than or at least 5, 10, 20, 30, 40, 50, 60, 70, 80, 90, 95, 97, 99, or 99.9% after administration of the effective amount of SRA737.

In some aspects, the present disclosure provides methods wherein the growth of the tumor is reduced at least 5, 10, 20, 30, 40, 50, 60, 70, 80, 90, 95, 97, 99, or 99.9% after administration of the effective amount of SRA737. In some aspects, the present disclosure provides methods wherein the growth of the tumor is reduced as measured by an increase or a decrease in total cell count in a MTT assay, or by change in genetic profile as measured by a ctDNA assay, by at least 5, 10, 20, 30, 40, 50, 60, 70, 80, 90, 95, 97, 99, or 99.9% after administration of the effective amount of SRA737.

In some aspects, the present disclosure provides methods wherein administration results in an IC₅₀ value below 10 μM and/or a GI₅₀ value below 1 μM. In some aspects, the present disclosure provides methods wherein administration results in an IC₅₀ value below 10 μM and/or a GI₅₀ value below 1 μM at twenty-four (24) hours after administration. In some aspects, the present disclosure provides methods wherein administration results in an IC₅₀ value below 10 μM and/or a GI₅₀ value below 1 μM at forty-eight (48) hours after administration.

In some aspects, the present disclosure provides methods wherein the administration results in an AUC of at least 1, 10, 25, 50, 100, 200, 400, 600, 800, or 1000.

In some aspects, the present disclosure provides methods wherein the administration results in an IC₅₀ value of no more than 0.001, 0.005, 0.01, 0.05, 0.1, 1, 3, 5, 10, 20, 40, 50, 60, 80, 90, 100, 200, 250, 300, 350, or 400 μM.

In some aspects, the present disclosure provides methods wherein the administration results in an EC₅₀ value of at least 0.01, 0.1, 1, 3, 5, 10, 20, 40, 50, 60, 80, 90, 100, 200, 250, 300, 350, or 400 μM.

In some aspects, the present disclosure provides methods wherein the administration results in an therapeutic index (TI) value ranging from about 1.001:1 to about 50:1, about 1.1:1 to about 15:1, about 1.2:1 to about 12:1, about 1.2:1 to about 10:1, about 1.2:1 to about 5:1, or about 1.2:1 to about 3:1.

In some aspects, the present disclosure provides methods wherein the administration results in an GI₅₀ value of at least 0.1 μM, 0.3 μM, 0.5 μM, 0.7 μM, 1 μM, 1.5 μM, 2 μM, 2.5 μM, 3 μM, 4 μM, 5 μM, or 10 μM.

In some aspects, the present disclosure provides methods wherein the administration results in a Maximum Response Observed (Max Response) value of no more than 0.1, 0.5, 1, 2 μM, 2.5 μM, 3 μM, 4 μM, 5 μM, or 10 μM.

Tumor growth can be expressed in terms of total tumor volume or total tumor size. There exist formulas, both generally speaking and specific to certain tumor models, that the skilled artisan can use to calculate tumor volume based upon the assumption that solid tumors are more or less spherical. In this regard, the skilled artisan can use experimental tools such as: ultrasound imaging, manual or digital calipers, ultrasonography, computed tomographic (CT), microCT, ¹⁸F-FDG-microPET, or magnetic resonance imaging (MRI) to measure tumor volume. See for example Monga S P, Wadleigh R, Sharma A, et al. Intratumoral therapy of cisplatin/epinephrine injectable gel for palliation in patients with obstructive esophageal cancer. Am. J. Clin. Oncol. 2000; 23(4):386-392; Mary M. Tomayko C., Patrick Reynolds, 1989. Determination of subcutaneous tumor size in athymic (nude) mice. Cancer Chemotherapy and Pharmacology, Volume 24, Issue 3, pp 148-154; E Richtig, G Langmann, K Mullner, G Richtig and J Smolle, 2004. Calculated tumour volume as a prognostic parameter for survival in choroidal melanomas. Eye (2004) 18, 619-623; Jensen et al. BMC Medical Imaging 2008. 8:16; Tomayko et al. Cancer Chemotherapy and Pharmacology September 1989, Volume 24, Issue 3, pp 148-154; and Faustino-Rocha et al. Lab Anim (NY). 2013 June; 42(6):217-24, each of which are hereby incorporated by reference in their entirety. In an illustrative example, tumor growth and/or size can be measured as a sum of the diameters (longest for non-nodal lesions, short axis for nodal lesions) for all target lesions and can be, in general, calculated and reported as the baseline sum diameters. The baseline sum diameters can be, in general, used as reference to further characterize any objective tumor regression in a measurable dimension of the disease.

In some aspects, the present disclosure provides methods wherein administration results in a reduction in tumor size, of at least 5, 10, 20, 30, 40, 50, 60, 70, 80, 90, 95, 97, 99 or 99.9% after administration of the effective amount of SRA737. In some aspects, the present disclosure provides methods wherein administration results in a reduction in tumor size of at least 30% after administration of the effective amount of SRA737. In some aspects, the present disclosure provides methods wherein administration results in a reduction in tumor volume of at least 5, 10, 20, 30, 40, 50, 60, 70, 80, 90, 95, 97, 99 or 99.9% after administration of the effective amount of SRA737. In some aspects, the present disclosure provides methods wherein administration results in a reduction in tumor volume of at least 30% after administration of the effective amount of SRA737. In some aspects, the present disclosure provides methods wherein administration results in a reduction in tumor volume or tumor size after one (1), two (2), three (3), four (4), six (6), eight (8), twelve (12), sixteen (16), twenty (20), twenty four (24), thirty six (36), or fifty two (52) weeks. In some aspects, the present disclosure provides methods wherein administration results in a reduction in tumor volume or tumor size of at least 30% after one (1), two (2), three (3), four (4), six (6), eight (8), twelve (12), sixteen (16), twenty (20), twenty four (24), thirty six (36), or fifty two (52) weeks. Reductions in tumor volume or tumor size can be measured by medical imaging techniques. Reductions in tumor volume or tumor size are, in general, determined relative to a baseline measurement.

Subjects

The present disclosure provides for administering an effective amount of SRA737 to a subject that is in need thereof. The present disclosure provides for administering an effective amount of SRA737 in a combination therapy with a further treatment to a subject that is in need thereof. In some aspects, the tumor from a subject is screened with genetic testing and/sequencing prior to administration. In some aspects, the tumor from a subject is screened with genetic testing and/sequencing after administration. In some aspects, the tumor from a subject is screened both after and before administration. In some aspects, healthy cells from the subject are screened with genetic testing and/sequencing prior to administration, after administration, or both. In some aspects, the tumor from a subject is screened with other biological tests or assays to determine the level of expression of certain biomarkers. In some aspects, the tumor from a subject is screened with both genetic testing and/sequencing and other biomarker tests or assays.

In some aspects, the present disclosure provides for methods wherein the subject is a mammal. In some aspects, the present disclosure provides for methods wherein the subject is a primate.

In some aspects, the present disclosure provides for methods wherein the subject is a mouse.

In some aspects, the present disclosure provides for methods wherein the subject is a human.

In some aspects, the present disclosure provides for methods wherein the subject is a human that has a tumor having a genetic mutation in one or more of the following genes: a tumor suppressor gene, a DNA damage repair gene, a replication stress gene, or an oncogenic driver gene. In some aspects, the present disclosure provides for methods wherein the subject is suffering from cancer in which the cancer cells have a genetic mutation in one or more of the following genes: a tumor suppressor gene, a DNA damage repair gene, a replication stress gene, or an oncogenic driver gene.

In some aspects, the present disclosure provides for methods wherein the tumor is in a human suffering from cancer that is selected from the group consisting of: colorectal cancer, ovarian cancer, high grade serous ovarian cancer (HGSOC), non-small cell lung cancer (NSCLC), small cell lung cancer, lung adenocarcinoma, prostate cancer, castration-resistant prostate cancer, bile duct cancer, cholangiocarcinoma, melanoma, uterine cancer, thyroid cancer, bladder cancer, breast cancer, cervical cancer, gastric cancer, endometrial cancer, hepatocellular cancer, leukemia, lymphoma, Non-Hodgkin's lymphoma, myeloma, brain cancer, neuroblastoma, squamous cell carcinoma, head and neck squamous cell carcinoma (HNSCC), and squamous cell carcinoma of the anus (SCCA), anogenital cancer (e.g., anal cancer), rectal cancer, pancreatic cancer, urothelial carcinoma, sarcoma and soft tissue sarcoma, metastatic colorectal cancer (CRC), platinum-resistant or intolerant HGSOC, advanced NSCLC, and metastatic castration-resistant prostate cancer (mCRPC), triple-negative breast cancer, invasive breast cancer, metastatic breast cancer, HER2 positive breast cancer and inflammatory breast cancer.

In some aspects, subjects have:
a. Histologically or cytologically proven advanced malignancy of the following types, for which no other conventional therapy is considered appropriate:
   i. High-grade serous ovarian cancer (HGSOC)
      1. Histologically confirmed high-grade serous ovarian, fallopian tube, or primary peritoneal cancer
      2. Platinum-resistant or refractory disease, or if the subject is intolerant to platinum therapy
   ii. Small cell lung cancer
      1. Subjects, in general, have received at least 1 but no more than 3 prior regimens for advanced disease, unless otherwise approved by sponsor
   iii. Soft tissue sarcoma
      1. Including undifferentiated pleiomorphic sarcoma/malignant fibrous histiocytoma (MFH) (including high-grade spindle cell sarcoma /pleomorphic liposarcomas), leiomyosarcoma, and dedifferentiated liposarcomas. Other types of STS may be eligible with sponsor's approval
      2. Subjects, in general, have received at least 1 but no more than 3 prior regimens for advanced disease, unless otherwise approved by sponsor
   iv. Cervical/anogenital cancer
      1. Including all cervical carcinoma and advanced/metastatic squamous cell carcinoma of the anus, penis, vagina, and vulva
      2. Subjects, in general, have received at least 1 but no more than 3 prior regimens for advanced disease, unless otherwise approved by sponsor
   v. Urothelial Carcinoma
      1. Histologically confirmed locally advanced and unresectable or metastatic urothelial carcinoma of the bladder, upper urinary tract or urethra
      2. Subjects, in general, have received at least 1 but no more than 3 prior regimens for advanced disease.
b. Measurable disease per RECIST v1.1 (see below)
c. Subjects have predicted sensitivity to Chk1 inhibition based on factors including: genetic profiling of tumor tissue or ctDNA, HPV status, and germline BRCA1 and BRCA2 gene status. All subjects have genetic profiling from tumor tissue or ctDNA; profiling is performed prospectively if required to evaluate Chk1 sensitivity or otherwise performed retrospectively
   i. For subjects with HGSOC, documented somatic or germline BRCA1 and BRCA2 wild-type status confer eligibility without requirement for prospective genetic profiling. If documented BRCA status is not available, genetic profiling may be performed prospectively to determine eligibility.
   ii. Subjects with SCLC are eligible without requirement for prospective genetic profiling on the basis of very high prevalence of cancer related alterations in the tumor suppressor genes (eg, TP53 and RB1) in this population.
   iii. For subjects with STS, and any others for whom genetic profiling is performed prospectively, eligibility is determined by the sponsor's review of genetic abnormalities detected in genes in the following categories:
      Key tumor suppressor genes regulating G1 cell cycle progression/arrest such as RB1, TP53, etc. For relevant cancers, positive human papilloma virus (HPV) status is also considered for eligibility.
         a. The DNA damage response pathway including ATM, BRCA1, BRCA2, mismatch repair genetic alterations and/or high microsatellite instability.
         b. Genetic indicators of replicative stress such as gain of function/amplification of Chk1 or ATR or other related gene.
         c. Oncogenic drivers such as MYC, CCNE1, etc.
   iv. For subjects with anogenital cancer, known HPV positive status will confer eligibility without requirement for prospective genetic profiling. If HPV status is not known or not positive, genetic profiling (or HPV testing where appropriate) may be performed prospectively to determine eligibility. Subjects with cervical cancer or squamous cell carcinoma of the anus are eligible without requirement for prospective genetic profiling based on the very high prevalence of HPV positivity in these populations.

In some aspects, subjects have one of the histologically or cytologically proven advanced malignancies described above and tumor tissue or ctDNA evidence that their tumor harbors one or more mutations that are expected to confer sensitivity to Chk1 inhibition. Eligibility can be determined by the sponsor's review of genetic abnormalities detected in genes in the following categories:
a. Key tumor suppressor genes regulating G1 cell cycle progression/arrest such as RB1, TP53, etc. For relevant cancers, positive human papilloma virus (HPV) status is also considered for eligibility.
b. The DNA damage response pathway including ATM, BRCA1, BRCA2, mismatch repair genetic alterations and/or high microsatellite instability.
c. Genetic indicators of replicative stress such as gain of function/amplification of Chk1 or ATR or other related gene.
d. Oncogenic drivers such as MYC, KRAS, etc.

In some aspects, subjects are excluded based on the following criteria:
a. Received the following prior or current anticancer therapy in the timeframes noted prior to receiving SRA737 and have recovered from toxicity:
   i. Radiotherapy, chemotherapy, PARP inhibitors, other targeted therapies, or other IMPs within 2 weeks
   ii. Nitrosoureas or Mitomycin C within 6 weeks
   iii. Any prior treatment with a Chk1 inhibitor at any point or prior treatment with an ATR inhibitor within 6 months
b. No more than 3 previous treatment regimens for advanced disease (not applicable to HGSOC expansion cohort)
c. Other malignancy within the past 2 years, except for adequately treated tumors
d. If, in the opinion of the Investigator, the subject is highly likely to experience clinically significant myelosuppression
e. Ongoing toxic manifestations of previous treatments greater than NCI-CTCAE Grade 1
f. History of allergy to gemcitabine
g. New or progressing brain metastases. Subjects with brain metastases that have been asymptomatic and radiologically stable over an 8-week period and have not been treated with steroids during that time may be included with approval from the sponsor.
h. High medical risk because of nonmalignant systemic disease
i. Serologically positive for hepatitis B, hepatitis C or HIV
j. Serious cardiac condition, left ventricular ejection fraction <45% at baseline, history of cardiac ischemia within the past 6 months, or prior history of cardiac arrhythmia requiring treatment, unless approved by the sponsor.
k. Prior bone marrow transplant or extensive radiotherapy to greater than 25% of bone marrow within the previous 8 weeks
l. Peanut allergy
m. QTcF >450 msec in adult makes and >470 msec in adult females
n. Impairment of gastrointestinal (GI) function or GI disease that may significantly alter the absorption of SRA737
o. Inability to swallow capsules without chewing or crushing
p. Is a participant or plans to participate in another interventional clinical trial
q. Any other condition which in the Investigator's opinion would not make the subject a good candidate

Administration

As disclosed herein, the methods of the invention include administration of the effective amount of SRA737. In an embodiment, the effective amount of SRA737 is administered as a monotherapy.

Also disclosed herein, the methods of the invention include a combination therapy administering an effective amount of SRA737 and coadministering a second effective amount of a further treatment. Further treatments include, but are not limited to, administering a chemotherapeutic agent, administering an antibody or antibody fragment (such as an immune checkpoint inhibitors), administering a radiation treatment, administering an external inducer of replication stress, and administering a combination thereof. Further treatments also include, but are not limited to, administering any one of gemcitabine, olaparib, niraparib, rucaparib, talazoparib, cisplatin, a ribonucleotide reductase inhibitor, etoposide, SN-38/CPT-11, mitomycin C, and combinations thereof. Coadministered encompasses methods where SRA737 and the further treatment are given simultaneously, where SRA737 and the further treatment are given sequentially, and where either one of, or both of, SRA737 and the further treatment are given intermittently or continuously, or any combination of: simultaneously, sequentially, intermittently and/or continuously. The skilled artisan will recognize that intermittent administration is not necessarily the same as sequential because intermittent also includes a first administration of an agent and then another administration later in time of that very same agent. Moreover, the skilled artisan understands that intermittent administration also encompasses sequential administration in some aspects because intermittent administration does include interruption of the first administration of an agent with an administration of a different agent before the first agent is administered again. Further, the skilled artisan will also know that continuous administration can be accomplished by a number of routes including i.v. drip or feeding tubes, etc.

Furthermore, and in a more general way, the term “coadministered” encompasses any and all methods where the individual administration of SRA737 and the individual administration of the further treatment to a subject overlap during any timeframe.

In one aspect, the frequency of administration of SRA737 or the further treatment to a subject includes, but is not limited to, Q1d, Q2d, Q3d, Q4d, Q5d, Q6d, Q7d, Q8d, Q9d, Q10d, Q14d, Q21d, Q28d, Q30d, Q90d, Q120d, Q240d, or Q365d. The term “QnD or qnd” refers to drug administration once every “n” days. For example, QD (or qd) refers to once every day or once daily dosing, Q2D (or q2d) refers to a dosing once every two days, Q7D refers to a dosing once every 7 days or once a week, Q5D refers to dosing once every 5 days, and so on. In one aspect, SRA737 and the further treatment are administered on different schedules.

In another aspect, the frequency of administration of SRA737 or the further treatment to a subject includes, but is not limited to 5 days of dosing followed by 2 days of non-dosing each week, 1 week of daily dosing followed by 1, 2, or 3 weeks of non-dosing, 2 or 3 weeks of daily dosing followed by 1, or 2 weeks of non-dosing, twice daily dosing; or dosing on days 2 and 3 of a weekly cycle. In one aspect, SRA737 and the further treatment are administered on different schedules.

In one aspect, the present disclosure provides methods where either one of, or all of, or any combination of SRA737 and/or at least one or more further treatment are administered intermittently. By intermittently is intended “noncontinuously.”

In one aspect, the present disclosure provides for methods comprising administering either one of, or both of, or any combinations thereof, SRA737 or the further treatment, to a subject with at least ten (10) minutes, fifteen (15) minutes, twenty (20) minutes, thirty (30) minutes, forty (40) minutes, sixty (60) minutes, two (2) hours, three (3) hour, four (4) hours, six (6) hours, eight (8) hours, ten (10) hours, twelve (12) hours, fourteen (14) hours, eighteen (18) hours, twenty-four (24) hours, thirty-six (36) hours, forty-eight (48) hours, three (3) days, four (4) days, five (5) days, six (6) days, seven (7) days, eight (8) days, nine (9) days, ten (10) days, eleven (11) days, twelve (12) days, thirteen (13) days, fourteen (14) days, three (3) weeks, or four (4) weeks, delay between administrations. In such aspects, the administration with a delay follows a pattern where one of or both of or any combination thereof SRA737 and/or the further treatment are administered continuously for a given period of time from about ten (10) minutes to about three hundred and sixty-five (365) days and then is not administered for a given period of time from about ten (10) minutes to about thirty (30) days. In one aspect, the present disclosure provides for methods where either one of or any combination of SRA737 and/or the further treatment are administered intermittently while the other is given continuously.

In one aspect, the present disclosure provides for methods where the combination of the effective amount of SRA737 is administered sequentially with the second effective amount of a further treatment.

In one aspect, the present disclosure provides for methods where SRA737 and the further treatment are administered simultaneously. In one aspect, the present disclosure provides for methods where the combination of the effective amount of SRA737 is administered sequentially with the second effective amount of a further treatment. In such aspects, the combination is also said to be “coadministered” since the term includes any and all methods where the subject is exposed to both components in the combination. However, such aspects are not limited to the combination being given just in one formulation or composition. In some cases, certain concentrations of SRA737 and the further treatment are more advantageous to deliver at certain intervals and as such, the effective amount of SRA737 and the second effective amount of the further treatment may change according to the formulation being administered.

In some aspects, the present disclosure provides for methods wherein SRA737 and at least one or more further treatment are administered simultaneously or sequentially. In some aspects, the present disclosure provides for methods where the effective amount of SRA737 is administered sequentially after the second effective amount of the further treatment. In some aspects, the present disclosure provides for methods where the second effective amount of the further treatment is administered sequentially after the effective amount of SRA737.

In some aspects, the present disclosure provides for methods where the combination is administered in one formulation. In some aspects, the present disclosure provides for methods where the combination is administered in two (2) compositions where the effective amount of SRA737 is administered in a separate formulation from the formulation of the second effective amount of the further treatment.

In some aspects, the present disclosure provides for methods where the effective amount of SRA737 is administered sequentially after the second effective amount of the further treatment. In some aspects, the present disclosure provides for methods where the second effective amount of the further treatment is administered sequentially after the effective amount of SRA737. In some aspects, the SRA737 and the further treatment are administered; and subsequently both SRA737 and the further treatment are administered intermittently for at least twenty-four (24) hours. In some aspects, SRA737 and the further treatment are administered on a non-overlapping every other day schedule. In some aspects, the further treatment is administered on day 1, and SRA737 is administered on days 2 and 3 of a weekly schedule.

In some aspects, the present disclosure provides for methods where the effective amount of SRA737 is administered no less than four (4) hours after the second effective amount of the further treatment. In one aspect, the present disclosure provides for methods where the effective amount of SRA737 is administered no less than ten (10) minutes, no less than fifteen (15) minutes, no less than twenty (20) minutes, no less than thirty (30) minutes, no less than forty (40) minutes, no less than sixty (60) minutes, no less than one (1) hour, no less than two (2) hours, no less than four (4) hours, no less than six (6) hours, no less than eight (8) hours, no less than ten (10) hours, no less than twelve (12) hours, no less than twenty four (24) hours, no less than two (2) days, no less than four (4) days, no less than six (6) days, no less than eight (8) days, no less than ten (10) days, no less than twelve (12) days, no less than fourteen (14) days, no less than twenty one (21) days, or no less than thirty (30) days after the second effective amount of the further treatment. In one aspect, the present disclosure provides for methods where the second effective amount of the further treatment is administered no less than ten (10) minutes, no less than fifteen (15) minutes, no less than twenty (20) minutes, no less than thirty (30) minutes, no less than forty (40) minutes, no less than sixty (60) minutes, no less than one (1) hour, no less than two (2) hours, no less than four (4) hours, no less than six (6) hours, no less than eight (8) hours, no less than ten (10) hours, no less than twelve (12) hours, no less than twenty four (24) hours, no less than two (2) days, no less than four (4) days, no less than six (6) days, no less than eight (8) days, no less than ten (10) days, no less than twelve (12) days, no less than fourteen (14) days, no less than twenty one (21) days, or no less than thirty (30) days after the effective amount of a SRA737.

In some aspects, the present disclosure provides for methods where either one of, or all of, or any combination thereof, SRA737 and/or at least one or more further treatment are administered by a route selected from the group consisting of: intravenous, subcutaneous, cutaneous, oral, intramuscular, and intraperitoneal. In some aspects, the present disclosure provides for methods where either one of, or both of, or any combination thereof, SRA737 and/or a further treatment are administered intravenously. In some aspects, the present disclosure provides for methods where either one of, or all of, or any combination thereof, SRA737 and/or at least one or more further treatment are administered orally.

In some embodiments, a second therapeutic agent (e.g., SRA737) is administered at least one hour after a first therapeutic agent (e.g., LDG) is administered. In some cases, a second therapeutic agent (e.g., SRA737) is administered at least two hours after a first therapeutic agent (e.g., LDG) is administered. In some cases, a second therapeutic agent (e.g., SRA737) is administered at least four hours after a first therapeutic agent (e.g., LDG) is administered. In some cases, a second therapeutic agent (e.g., SRA737) is administered at least six hours after a first therapeutic agent (e.g., LDG) is administered. In some cases, a second therapeutic agent (e.g., SRA737) is administered at least eight hours after a first therapeutic agent (e.g., LDG) is administered. In some cases, a second therapeutic agent (e.g., SRA737) is administered at least ten hours after a first therapeutic agent (e.g., LDG) is administered. In some cases, a second therapeutic agent (e.g., SRA737) is administered at least twelve hours after a first therapeutic agent (e.g., LDG) is administered. In some cases, a second therapeutic agent (e.g., SRA737) is administered at least fourteen hours after a first therapeutic agent (e.g., LDG) is administered. In some cases, a second therapeutic agent (e.g., SRA737) is administered at least sixteen hours after a first therapeutic agent (e.g., LDG) is administered. In some cases, a second therapeutic agent (e.g., SRA737) is administered at least eighteen hours after a first therapeutic agent (e.g., LDG) is administered. In some cases, a second therapeutic agent (e.g., SRA737) is administered at least twenty hours after a first therapeutic agent (e.g., LDG) is administered. In some cases, a second therapeutic agent (e.g., SRA737) is administered at least twenty-two hours after a first therapeutic agent (e.g., LDG) is administered. Similarly, a second therapeutic agent (SRA737) is administered at least twenty-four hours, or 48 hours after a first therapeutic agent (e.g. LDG) is administered.

In some embodiments, a third therapeutic agent (e.g., an immune checkpoint inhibitor) is administered at least one hour after a second therapeutic agent (e.g., SRA737) is administered. In some cases, a third therapeutic agent (e.g., an immune checkpoint inhibitor) is administered at least two hours after a second therapeutic agent (e.g., SRA737) is administered. In some cases, a third therapeutic agent (e.g., an immune checkpoint inhibitor) is administered at least four hours after a second therapeutic agent (e.g., SRA737) is administered. In some cases, a third therapeutic agent (e.g., an immune checkpoint inhibitor) is administered at least six hours after a second therapeutic agent (e.g., SRA737) is administered. In some cases, a third therapeutic agent (e.g., an immune checkpoint inhibitor) is administered at least eight hours after a second therapeutic agent (e.g., SRA737) is administered. In some cases, a third therapeutic agent (e.g., an immune checkpoint inhibitor) is administered at least ten hours after a second therapeutic agent (e.g., SRA737) is administered. In some cases, a third therapeutic agent (e.g., an immune checkpoint inhibitor) is administered at least twelve hours after a second therapeutic agent (e.g., SRA737) is administered. In some cases, a third therapeutic agent (e.g., an immune checkpoint inhibitor) is administered at least fourteen hours after a second therapeutic agent (e.g., SRA737) is administered. In some cases, a third therapeutic agent (e.g., an immune checkpoint inhibitor) is administered at least sixteen hours after a second therapeutic agent (e.g., SRA737) is administered. In some cases, a third therapeutic agent (e.g., an immune checkpoint inhibitor) is administered at least eighteen hours after a second therapeutic agent (e.g., SRA737) is administered. In some cases, a third therapeutic agent (e.g., an immune checkpoint inhibitor) is administered at least twenty hours after a second therapeutic agent (e.g., SRA737) is administered. In some cases, a third therapeutic agent (e.g., an immune checkpoint inhibitor) is administered at least twenty-two hours after a second therapeutic agent (e.g., SRA737) is administered. In some cases, a third therapeutic agent (e.g., an immune checkpoint inhibitor) is administered at least twenty-four hours or 48 hours after a second therapeutic agent (e.g., SRA737) is administered.

In some embodiments, a second therapeutic agent (e.g., SRA737) is administered at least one hour after a first therapeutic agent (e.g., LDG) is administered, and a third therapeutic agent (e.g. an immune checkpoint inhibitor) is administered at least one hour after the second therapeutic agent is administered.

In some embodiments, a second therapeutic agent (e.g., SRA737) is administered at least two hours after a first therapeutic agent (e.g., LDG) is administered, and a third therapeutic agent (e.g. an immune checkpoint inhibitor) is administered at least one hour after the second therapeutic agent is administered.

In some embodiments, a second therapeutic agent (e.g., SRA737) is administered at least four hours after a first therapeutic agent (e.g., LDG) is administered, and a third therapeutic agent (e.g. an immune checkpoint inhibitor) is administered at least one hour after the second therapeutic agent is administered.

In some embodiments, a second therapeutic agent (e.g., SRA737) is administered at least six hours after a first therapeutic agent (e.g., LDG) is administered, and a third therapeutic agent (e.g. an immune checkpoint inhibitor) is administered at least one hour after the second therapeutic agent is administered.

In some embodiments, a second therapeutic agent (e.g., SRA737) is administered at least eight hours after a first therapeutic agent (e.g., LDG) is administered, and a third therapeutic agent (e.g. an immune checkpoint inhibitor) is administered at least one hour after the second therapeutic agent is administered.

In some embodiments, a second therapeutic agent (e.g., SRA737) is administered at least ten hours after a first therapeutic agent (e.g., LDG) is administered, and a third therapeutic agent (e.g., an immune checkpoint inhibitor) is administered at least one hour after the second therapeutic agent is administered.

In some embodiments, a second therapeutic agent (e.g., SRA737) is administered at least twelve hours after a first therapeutic agent (e.g., LDG) is administered, and a third therapeutic agent (e.g., an immune checkpoint inhibitor) is administered at least one hour after the second therapeutic agent is administered.

In some embodiments, a second therapeutic agent (e.g., SRA737) is administered at least fourteen hours after a first therapeutic agent (e.g., LDG) is administered, and a third therapeutic agent (e.g., an immune checkpoint inhibitor) is administered at least one hour after the second therapeutic agent is administered.

In some embodiments, a second therapeutic agent (e.g., SRA737) is administered at least sixteen hours after a first therapeutic agent (e.g., LDG) is administered, and a third therapeutic agent (e.g., an immune checkpoint inhibitor) is administered at least one hour after the second therapeutic agent is administered.

In some embodiments, a second therapeutic agent (e.g., SRA737) is administered at least eighteen hours after a first therapeutic agent (e.g., LDG) is administered, and a third therapeutic agent (e.g., an immune checkpoint inhibitor) is administered at least one hour after the second therapeutic agent is administered.

In some embodiments, a second therapeutic agent (e.g., SRA737) is administered at least twenty hours after a first therapeutic agent (e.g., LDG) is administered, and a third therapeutic agent (e.g., an immune checkpoint inhibitor) is administered at least one hour after the second therapeutic agent is administered.

In some embodiments, a second therapeutic agent (e.g., SRA737) is administered at least twenty-two hours after a first therapeutic agent (e.g., LDG) is administered, and a third therapeutic agent (e.g., an immune checkpoint inhibitor) is administered at least one hour after the second therapeutic agent is administered.

In some embodiments, a second therapeutic agent (e.g., SRA737) is administered at least twenty-four hours after a first therapeutic agent (e.g., LDG) is administered, and a third therapeutic agent (e.g., an immune checkpoint inhibitor) is administered at least one hour after the second therapeutic agent is administered.

In some embodiments, a second therapeutic agent (e.g., SRA737) is administered at least one hour after a first therapeutic agent (e.g., LDG) is administered, and a third therapeutic agent (e.g., an immune checkpoint inhibitor) is administered at least one hour after the second therapeutic agent is administered.

In some embodiments, a second therapeutic agent (e.g., SRA737) is administered at least one hour after a first therapeutic agent (e.g., LDG) is administered, and a third therapeutic agent (e.g., an immune checkpoint inhibitor) is administered at least two hours after the second therapeutic agent is administered.

In some embodiments, a second therapeutic agent (e.g., SRA737) is administered at least one hour after a first therapeutic agent (e.g., LDG) is administered, and a third therapeutic agent (e.g., an immune checkpoint inhibitor) is administered at least four hours after the second therapeutic agent is administered.

In some embodiments, a second therapeutic agent (e.g., SRA737) is administered at least one hour after a first therapeutic agent (e.g., LDG) is administered, and a third therapeutic agent (e.g., an immune checkpoint inhibitor) is administered at least six hours after the second therapeutic agent is administered.

In some embodiments, a second therapeutic agent (e.g., SRA737) is administered at least one hour after a first therapeutic agent (e.g., LDG) is administered, and a third therapeutic agent (e.g., an immune checkpoint inhibitor) is administered at least eight hours after the second therapeutic agent is administered.

In some embodiments, a second therapeutic agent (e.g., SRA737) is administered at least one hour after a first therapeutic agent (e.g., LDG) is administered, and a third therapeutic agent (e.g., an immune checkpoint inhibitor) is administered at least ten hours after the second therapeutic agent is administered.

In some embodiments, a second therapeutic agent (e.g., SRA737) is administered at least one hour after a first therapeutic agent (e.g., LDG) is administered, and a third therapeutic agent (e.g., an immune checkpoint inhibitor) is administered at least twelve hours after the second therapeutic agent is administered.

In some embodiments, a second therapeutic agent (e.g., SRA737) is administered at least one hour after a first therapeutic agent (e.g., LDG) is administered, and a third therapeutic agent (e.g., an immune checkpoint inhibitor) is administered at least fourteen hours after the second therapeutic agent is administered.

In some embodiments, a second therapeutic agent (e.g., SRA737) is administered at least one hour after a first therapeutic agent (e.g., LDG) is administered, and a third therapeutic agent (e.g., an immune checkpoint inhibitor) is administered at least sixteen hours after the second therapeutic agent is administered.

In some embodiments, a second therapeutic agent (e.g., SRA737) is administered at least one hour after a first therapeutic agent (e.g., LDG) is administered, and a third therapeutic agent (e.g., an immune checkpoint inhibitor) is administered at least eighteen hours after the second therapeutic agent is administered.

In some embodiments, a second therapeutic agent (e.g., SRA737) is administered at least one hour after a first therapeutic agent (e.g., LDG) is administered, and a third therapeutic agent (e.g., an immune checkpoint inhibitor) is administered at least twenty hours after the second therapeutic agent is administered.

In some embodiments, a second therapeutic agent (e.g., SRA737) is administered at least one hour after a first therapeutic agent (e.g., LDG) is administered, and a third therapeutic agent (e.g., an immune checkpoint inhibitor) is administered at least twenty-two hours after the second therapeutic agent is administered.

In some embodiments, a second therapeutic agent (e.g., SRA737) is administered at least one hour after a first therapeutic agent (e.g., LDG) is administered, and a third therapeutic agent (e.g., an immune checkpoint inhibitor) is administered at least twenty-four hours after the second therapeutic agent is administered.

In some embodiments, a second therapeutic agent (e.g., SRA737) is administered at least one hour after a first therapeutic agent (e.g., LDG) is administered, and a third therapeutic agent (e.g., an immune checkpoint inhibitor) is administered at least 48 hours after the second therapeutic agent is administered.

In another aspect, a second therapeutic agent (e.g., SRA737) is administered at the same time as a third therapeutic agent (e.g., an immune checkpoint inhibitor) at least one hour after a first therapeutic agent (e.g., LDG) is administered.

In some embodiments, a second therapeutic agent (e.g., SRA737) is administered at the same time as a third therapeutic agent (e.g., an immune checkpoint inhibitor) at least two hours, four hours, six hours, eight hours, ten hours, twelve hours, fourteen hours, sixteen hours, eighteen hours, twenty hours, twenty-two hours, twenty-four hours, or 48 hours after a first therapeutic agent (e.g., LDG) is administered.

It is understood by the skilled artisan that the unit dose forms of the present disclosure may be administered in the same or different physicals forms, i.e. orally via capsules or tablets and/or by liquid via i.v. infusion, and so on. Moreover, the unit dose forms for each administration may differ by the particular route of administration. Several various dosage forms may exist for either one of, or both of, or all three of SRA737, LDG, and an immune checkpoint inhibitor treatments. Because different medical conditions can warrant different routes of administration, the same components of a combination of SRA737, LDG and an immune checkpoint inhibitor described herein may be exactly alike in composition and physical form and yet may need to be given in differing ways and perhaps at differing times to alleviate the condition being treated in a subject. For example, a condition such as persistent nausea, especially with vomiting, can make it difficult to use an oral dosage form, and in such a case, it may be necessary to administer another unit dose form, perhaps even one identical to other dosage forms used previously or afterward, with an inhalation, buccal, sublingual, or suppository route instead or as well. The specific dosage form may be a requirement for certain combinations of SRA737, a further treatment and a third treatment, as there may be issues with various factors like chemical stability or pharmacokinetics.

Therapeutically Effective Amount and Unit Dose Form

The present disclosure provides for a method of treatment wherein the effective amount of SRA737 is administered to a subject. The term “effective amount” or “therapeutically effective amount” refers to an amount that is effective to ameliorate a symptom of a disease, e.g. an amount that is effective to inhibit the growth of a tumor. In some aspects, the effective amount of SRA737 is less than or equal to the maximum tolerated dose (MTD), less than or equal to the highest non-severely toxic dose (HNSTD), or less than or equal to the No-observed-adverse-effect-level (NOAEL). In some aspects, the effective amount of SRA737 is less than 2000 mg/day orally. In some aspects, the effective amount of SRA737 is less than 1500 mg/day orally. In some aspects, the effective amount of SRA737 is less than 1300 mg/day orally. In some aspects, the effective amount of SRA737 is greater than 600 mg/day orally. In some aspects, the effective amount of SRA737 is between 600-2000 mg/day orally. In some aspects, the effective amount of SRA737 is between 600-1500 mg/day orally. In some aspects, the effective amount of SRA737 is between 600-1300 mg/day orally. In some aspects, the effective amount of SRA737 is between 600-1000 mg/day orally. In some aspects, the effective amount of SRA737 is 600 mg/day, 700 mg/day, 800 mg/day, 900 mg/day, 1000 mg/day, 1100 mg/day, 1200 mg/day, 1300 mg/day, 1500 mg/day, or 2000 mg/day orally.

In specific embodiments of the invention, the effective amount of SRA737 is administered to a subject as a monotherapy. In some aspects, the effective amount of the SRA737 monotherapy is less than or equal to the maximum tolerated dose (MTD), less than 0 or equal to the highest non-severely toxic dose (HNSTD), or less than or equal to the No-observed-adverse-effect-level (NOAEL). In some aspects, the effective amount of the SRA737 monotherapy is less than 2000 mg/day orally. In some aspects, the effective amount of the SRA737 monotherapy is less than 1500 mg/day orally. In some aspects, the effective amount of the SRA737 monotherapy is less than 1300 mg/day orally. In some aspects, the effective amount of the SRA737 monotherapy is greater than 600 mg/day orally. In some aspects, the effective amount of the SRA737 monotherapy is between 600-2000 mg/day orally. In some aspects, the effective amount of the SRA737 monotherapy is between 600-1500 mg/day orally. In some aspects, the effective amount of the SRA737 monotherapy is between 600-1300 mg/day orally. In some aspects, the effective amount of the SRA737 monotherapy is between 600-1000 mg/day orally. In some aspects, the effective amount of the SRA737 monotherapy is 600 mg/day, 700 mg/day, 800 mg/day, 900 mg/day, 1000 mg/day, 1100 mg/day, 1200 mg/day, 1300 mg/day, 1500 mg/day, or 2000 mg/day orally. In some aspects, the effective amount of the SRA737 monotherapy is 600 mg/day. In some aspects, the effective amount of the SRA737 monotherapy is 700 mg/day. In some aspects, the effective amount of the SRA737 monotherapy is 800 mg/day. In some aspects, the effective amount of the SRA737 monotherapy is 900 mg/day. In some aspects, the effective amount of the SRA737 monotherapy is 1000 mg/day. In some aspects, the effective amount of the SRA737 monotherapy is 1100 mg/day. In some aspects, the effective amount of the SRA737 monotherapy is 1200 mg/day. In some aspects, the effective amount of the SRA737 monotherapy is 1300 mg/day. In some aspects, the effective amount of the SRA737 monotherapy is 1500 mg/day. In some aspects, the effective amount of the SRA737 monotherapy is or 2000 mg/day.

In specific embodiments of the invention, the effective amount of SRA737 is administered to a subject as a combination therapy. In some aspects, the effective amount of the SRA737 combination therapy is less than or equal to the maximum tolerated dose (MTD), less than or equal to the highest non-severely toxic dose (HNSTD), or less than or equal to the No-observed-adverse-effect-level (NOAEL). In some aspects, the effective amount of the SRA737 combination therapy is less than the effective amount of the SRA737 monotherapy. In some aspects, the effective amount of the SRA737 combination therapy is less than 2000 mg/day orally. In some aspects, the effective amount of the SRA737 combination therapy is less than 1500 mg/day orally. In some aspects, the effective amount of the SRA737 combination therapy is less than 1300 mg/day orally. In some aspects, the effective amount of the SRA737 combination therapy is 600 mg/day or less orally. In some aspects, the effective amount of the SRA737 combination therapy is at least 300 mg/day orally. In some aspects, the effective amount of the SRA737 combination therapy is at least 100 mg/day orally. In some aspects, the effective amount of the SRA737 combination therapy is at least 600 mg/day orally. In some aspects, the effective amount of the SRA737 combination therapy is between 100-2000 mg/day orally. In some aspects, the effective amount of the SRA737 combination therapy is between 300-2000 mg/day orally. In some aspects, the effective amount of the SRA737 combination therapy is between 600-2000 mg/day orally. In some aspects, the effective amount of the SRA737 combination therapy is between 300-1500 mg/day orally. In some aspects, the effective amount of the SRA737 combination therapy is between 300-1300 mg/day orally. In some aspects, the effective amount of the SRA737 combination therapy is between 300-1000 mg/day orally. In some aspects, the effective amount of the SRA737 combination therapy is 100 mg/day, 150 mg/day, 200 mg/day, 300 mg/day, 600 mg/day, 700 mg/day, 800 mg/day, 900 mg/day, 1000 mg/day, 1100 mg/day, 1200 mg/day, 1300 mg/day, 1500 mg/day, or 2000 mg/day orally. In some aspects, the effective amount of the SRA737 combination therapy is 300 mg/day, 400 mg/day, 500 mg/day, 600 mg/day, 700 mg/day, 800 mg/day, 900 mg/day, 1000 mg/day, 1100 mg/day, 1200 mg/day, 1300 mg/day, 1500 mg/day, or 2000 mg/day orally.

In some aspects, the effective amount of the SRA737 combination therapy is 300 mg/day. In some aspects, the effective amount of the SRA737 combination therapy is 400 mg/day. In some aspects, the effective amount of the SRA737 combination therapy is 500 mg/day. In some aspects, the effective amount of the SRA737 combination therapy is 600 mg/day. In some aspects, the effective amount of the SRA737 combination therapy is 700 mg/day. In some aspects, the effective amount of the SRA737 combination therapy is 800 mg/day. In some aspects, the effective amount of the SRA737 combination therapy is 900 mg/day. In some aspects, the effective amount of the SRA737 combination therapy is 1000 mg/day. In some aspects, the effective amount of the SRA737 combination therapy is 1100 mg/day. In some aspects, the effective amount of the SRA737 combination therapy is 1200 mg/day. In some aspects, the effective amount of the SRA737 combination therapy is at least 300 mg/day. In some aspects, the effective amount of the SRA737 combination therapy is at least 400 mg/day. In some aspects, the effective amount of the SRA737 combination therapy is at least 500 mg/day. In some aspects, the effective amount of the SRA737 combination therapy is at least 600 mg/day. In some aspects, the effective amount of the SRA737 combination therapy is at least 700 mg/day. In some aspects, the effective amount of the SRA737 combination therapy is at least 800 mg/day. In some aspects, the effective amount of the SRA737 combination therapy is at least 900 mg/day. In some aspects, the effective amount of the SRA737 combination therapy is at least 1000 mg/day. In some aspects, the effective amount of the SRA737 combination therapy is at least 1100 mg/day. In some aspects, the effective amount of the SRA737 combination therapy is at least 1200 mg/day. In some aspects, the effective amount of the SRA737 combination therapy is 300 mg/day or less. In some aspects, the effective amount of the SRA737 combination therapy is 400 mg/day or less. In some aspects, the effective amount of the SRA737 combination therapy is 500 mg/day or less. In some aspects, the effective amount of the SRA737 combination therapy is 600 mg/day or less. In some aspects, the effective amount of the SRA737 combination therapy is 700 mg/day or less. In some aspects, the effective amount of the SRA737 combination therapy is 800 mg/day or less. In some aspects, the effective amount of the SRA737 combination therapy is 900 mg/day or less. In some aspects, the effective amount of the SRA737 combination therapy is 1000 mg/day or less. In some aspects, the effective amount of the SRA737 combination therapy is 1100 mg/day or less. In some aspects, the effective amount of the SRA737 combination therapy is 1200 mg/day or less.

In some aspects, the effective amount of the SRA737 combination therapy is 350 mg/day. In some aspects, the effective amount of the SRA737 combination therapy is 450 mg/day. In some aspects, the effective amount of the SRA737 combination therapy is 550 mg/day. In some aspects, the effective amount of the SRA737 combination therapy is 650 mg/day. In some aspects, the effective amount of the SRA737 combination therapy is 750 mg/day. In some aspects, the effective amount of the SRA737 combination therapy is 850 mg/day. In some aspects, the effective amount of the SRA737 combination therapy is 950 mg/day. In some aspects, the effective amount of the SRA737 combination therapy is 1050 mg/day. In some aspects, the effective amount of the SRA737 combination therapy is 1150 mg/day. In some aspects, the effective amount of the SRA737 combination therapy is 1250 mg/day. In some aspects, the effective amount of the SRA737 combination therapy is at least 350 mg/day. In some aspects, the effective amount of the SRA737 combination therapy is at least 450 mg/day. In some aspects, the effective amount of the SRA737 combination therapy is at least 550 mg/day. In some aspects, the effective amount of the SRA737 combination therapy is at least 650 mg/day. In some aspects, the effective amount of the SRA737 combination therapy is at least 750 mg/day. In some aspects, the effective amount of the SRA737 combination therapy is at least 850 mg/day. In some aspects, the effective amount of the SRA737 combination therapy is at least 950 mg/day. In some aspects, the effective amount of the SRA737 combination therapy is at least 1050 mg/day. In some aspects, the effective amount of the SRA737 combination therapy is at least 1150 mg/day. In some aspects, the effective amount of the SRA737 combination therapy is at least 1250 mg/day. In some aspects, the effective amount of the SRA737 combination therapy is 350 mg/day or less. In some aspects, the effective amount of the SRA737 combination therapy is 450 mg/day or less. In some aspects, the effective amount of the SRA737 combination therapy is 550 mg/day or less. In some aspects, the effective amount of the SRA737 combination therapy is 650 mg/day or less. In some aspects, the effective amount of the SRA737 combination therapy is 750 mg/day or less. In some aspects, the effective amount of the SRA737 combination therapy is 850 mg/day or less. In some aspects, the effective amount of the SRA737 combination therapy is 950 mg/day or less. In some aspects, the effective amount of the SRA737 combination therapy is 1050 mg/day or less. In some aspects, the effective amount of the SRA737 combination therapy is 1150 mg/day or less. In some aspects, the effective amount of the SRA737 combination therapy is 1250 mg/day or less.

In some aspects, the effective amount of the SRA737 combination therapy is 325 mg/day. In some aspects, the effective amount of the SRA737 combination therapy is 425 mg/day. In some aspects, the effective amount of the SRA737 combination therapy is 525 mg/day. In some aspects, the effective amount of the SRA737 combination therapy is 625 mg/day. In some aspects, the effective amount of the SRA737 combination therapy is 725 mg/day. In some aspects, the effective amount of the SRA737 combination therapy is 825 mg/day. In some aspects, the effective amount of the SRA737 combination therapy is 925 mg/day. In some aspects, the effective amount of the SRA737 combination therapy is 1025 mg/day. In some aspects, the effective amount of the SRA737 combination therapy is 1125 mg/day. In some aspects, the effective amount of the SRA737 combination therapy is 1225 mg/day. In some aspects, the effective amount of the SRA737 combination therapy is at least 325 mg/day. In some aspects, the effective amount of the SRA737 combination therapy is at least 425 mg/day. In some aspects, the effective amount of the SRA737 combination therapy is at least 525 mg/day. In some aspects, the effective amount of the SRA737 combination therapy is at least 625 mg/day. In some aspects, the effective amount of the SRA737 combination therapy is at least 725 mg/day. In some aspects, the effective amount of the SRA737 combination therapy is at least 825 mg/day. In some aspects, the effective amount of the SRA737 combination therapy is at least 925 mg/day. In some aspects, the effective amount of the SRA737 combination therapy is at least 1025 mg/day. In some aspects, the effective amount of the SRA737 combination therapy is at least 1125 mg/day. In some aspects, the effective amount of the SRA737 combination therapy is at least 1225 mg/day. In some aspects, the effective amount of the SRA737 combination therapy is 325 mg/day or less. In some aspects, the effective amount of the SRA737 combination therapy is 425 mg/day or less. In some aspects, the effective amount of the SRA737 combination therapy is 525 mg/day or less. In some aspects, the effective amount of the SRA737 combination therapy is 625 mg/day or less. In some aspects, the effective amount of the SRA737 combination therapy is 725 mg/day or less. In some aspects, the effective amount of the SRA737 combination therapy is 825 mg/day or less. In some aspects, the effective amount of the SRA737 combination therapy is 925 mg/day or less. In some aspects, the effective amount of the SRA737 combination therapy is 1025 mg/day or less. In some aspects, the effective amount of the SRA737 combination therapy is 1125 mg/day or less. In some aspects, the effective amount of the SRA737 combination therapy is 1225 mg/day or less.

In some aspects, the effective amount of the SRA737 combination therapy is 375 mg/day. In some aspects, the effective amount of the SRA737 combination therapy is 475 mg/day. In some aspects, the effective amount of the SRA737 combination therapy is 575 mg/day. In some aspects, the effective amount of the SRA737 combination therapy is 675 mg/day. In some aspects, the effective amount of the SRA737 combination therapy is 775 mg/day. In some aspects, the effective amount of the SRA737 combination therapy is 875 mg/day. In some aspects, the effective amount of the SRA737 combination therapy is 975 mg/day. In some aspects, the effective amount of the SRA737 combination therapy is 1075 mg/day. In some aspects, the effective amount of the SRA737 combination therapy is 1175 mg/day. In some aspects, the effective amount of the SRA737 combination therapy is 1275 mg/day. In some aspects, the effective amount of the SRA737 combination therapy is at least 375 mg/day. In some aspects, the effective amount of the SRA737 combination therapy is at least 475 mg/day. In some aspects, the effective amount of the SRA737 combination therapy is at least 575 mg/day. In some aspects, the effective amount of the SRA737 combination therapy is at least 675 mg/day. In some aspects, the effective amount of the SRA737 combination therapy is at least 775 mg/day. In some aspects, the effective amount of the SRA737 combination therapy is at least 875 mg/day. In some aspects, the effective amount of the SRA737 combination therapy is at least 975 mg/day. In some aspects, the effective amount of the SRA737 combination therapy is at least 1075 mg/day. In some aspects, the effective amount of the SRA737 combination therapy is at least 1175 mg/day. In some aspects, the effective amount of the SRA737 combination therapy is at least 1275 mg/day. In some aspects, the effective amount of the SRA737 combination therapy is 375 mg/day or less. In some aspects, the effective amount of the SRA737 combination therapy is 475 mg/day or less. In some aspects, the effective amount of the SRA737 combination therapy is 575 mg/day or less. In some aspects, the effective amount of the SRA737 combination therapy is 675 mg/day or less. In some aspects, the effective amount of the SRA737 combination therapy is 775 mg/day or less. In some aspects, the effective amount of the SRA737 combination therapy is 875 mg/day or less. In some aspects, the effective amount of the SRA737 combination therapy is 975 mg/day or less. In some aspects, the effective amount of the SRA737 combination therapy is 1075 mg/day or less. In some aspects, the effective amount of the SRA737 combination therapy is 1175 mg/day or less. In some aspects, the effective amount of the SRA737 combination therapy is 1275 mg/day or less.

In specific embodiments of the invention, the effective amount of SRA737 is administered to a subject as a combination therapy with a second effective amount of at least one or more further treatment. In some aspects, the second effective amount is an amount from about 0.001 mg/kg to about 15 mg/kg. In some embodiments the second effective amount of the further treatment is 0.001, 0.005, 0.010, 0.020, 0.050, 0.1, 0.2, 0.5, 1.0, 2.0, 5.0, 10.0 or 15.0 mg/kg. In some embodiments the second effective amount of the further treatment is between 10-2000 mg/m²/day. In some embodiments the second effective amount of the further treatment is between 50-1250 mg/m²/day. In some embodiments the second effective amount of the further treatment is 50 mg/m²/day, 100 mg/m²/day, 150 mg/m²/day, 200 mg/m²/day, 250 mg/m²/day, 300 mg/m²/day, 350 mg/m²/day, 400 mg/m²/day, 450 mg/m²/day, 500 mg/m²/day, 550 mg/m²/day, 600 mg/m²/day, 650 mg/m²/day, 700 mg/m²/day, 750 mg/m²/day, 800 mg/m²/day, 850 mg/m²/day, 900 mg/m²/day, 950 mg/m²/day, 1000 mg/m²/day, 1050 mg/m²/day, 1100 mg/m²/day, 1150 mg/m²/day, 1200 mg/m²/day, or 1250 mg/m²/day.

In other specific embodiments of the invention, the effective amount of SRA737 is administered to a subject as a combination therapy with a second effective amount of a further treatment and a third effective amount of a third treatment. In some embodiments, the third effective amount of the third treatment is an amount from about 0.001 mg to about 10 mg. In some embodiments, the third effective amount of the third treatment is at least 0.01 mg. In some embodiments, the third effective amount of the third treatment is at least 0.02 mg. In some embodiments, the third effective amount of the third treatment is at least 0.03 mg. In some embodiments, the third effective amount of the third treatment is at least 0.04 mg. In some embodiments, the third effective amount of the third treatment is at least 0.05 mg. In some embodiments, the third effective amount of the third treatment is at least 0.06 mg. In some embodiments, the third effective amount of the third treatment is at least 0.07 mg. In some embodiments, the third effective amount of the third treatment is at least 0.08 mg. In some embodiments, the third effective amount of the third treatment is at least 0.09 mg. In some embodiments, the third effective amount of the third treatment is at least 0.1 mg, 0.2 mg, 0.3 mg, 0.4 mg, 0.5 mg, 0.6 mg, 0.7 mg, 0.8 mg, 0.9 mg, 1.0 mg, 1.2 mg, 1.4 mg, 1.6 mg, 1.8 mg, 2.0 mg, 2.5 mg, 3.0 mg, 3.5 mg, 4.0 mg, 5.0 mg, 6.0 mg, 7.0 mg, 8.0 mg, 9.0 mg or 10.0 mg. In some embodiments, the third effective amount of the third treatment is an amount from about 0.001 mg/kg to about 15 mg/kg. In some embodiments, the third effective amount of the third treatment is 0.001, 0.005, 0.010, 0.020, 0.050, 0.1, 0.2, 0.5, 1.0, 2.0, 5.0, 10.0 or 15.0 mg/kg. In some embodiments, the third effective amount of the third treatment is between 10-2000 mg/m²/day. In some embodiments the third effective amount of the third treatment is between 50-1250 mg/m²/day. In some embodiments the third effective amount of the third treatment is 50 mg/m²/day, 100 mg/m²/day, 150 mg/m²/day, 200 mg/m²/day, 250 mg/m²/day, 300 mg/m²/day, 350 mg/m²/day, 400 mg/m²/day, 450 mg/m²/day, 500 mg/m²/day, 550 mg/m²/day, 600 mg/m²/day, 650 mg/m²/day, 700 mg/m²/day, 750 mg/m²/day, 800 mg/m²/day, 850 mg/m²/day, 900 mg/m²/day, 950 mg/m²/day, 1000 mg/m²/day, 1050 mg/m²/day, 1100 mg/m²/day, 1150 mg/m²/day, 1200 mg/m²/day, or 1250 mg/m²/day.

In general, the compounds of the present technology will be administered in a therapeutically effective amount by any of the accepted modes of administration for agents that serve similar utilities. the actual amount of the compound of the present technology, i.e., the active ingredient, will depend upon numerous factors such as the severity of the disease to be treated, the age and relative health of the subject, the potency of the compound used, the route and form of administration, and other factors well known to the skilled artisan. The drug can be administered at least once a day, preferably once or twice a day.

An effective amount of such agents can readily be determined by routine experimentation, as can the most effective and convenient route of administration and the most appropriate formulation. Various formulations and drug delivery systems are available in the art. See, e.g., Gennaro, A. R., ed. (1995) Remington's Pharmaceutical Sciences, 18th ed., Mack Publishing Co.

A therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

An effective amount or a therapeutically effective amount or dose of an agent, e.g., a compound of the present technology, refers to that amount of the agent or compound that results in amelioration of symptoms or a prolongation of survival in a subject. Toxicity and therapeutic efficacy of such molecules can be determined by standard pharmaceutical procedures in cell cultures or experimental animals, e.g., by determining the maximum tolerated dose (MTD), the highest non-severely toxic dose (HNSTD), the No-observed-adverse-effect-level (NOAEL), or the LD₅₀ (the dose lethal to 50% of the population) and the ED₅₀ (the dose therapeutically effective in 50% of the population). the dose ratio of toxic to therapeutic effects is therapeutic index, which can be expressed as the ratio of the MTD, HNSTD, NOAEL, or LD₅₀ to the ED₅₀. Agents that exhibit high therapeutic indices are preferred.

The effective amount or therapeutically effective amount is the amount of the compound or pharmaceutical composition that will elicit the biological or medical response of a tissue, system, animal or human that is being sought by the researcher, veterinarian, medical doctor or other clinician. Dosages particularly fall within a range of circulating concentrations that includes the ED₅₀ with little or no toxicity. Dosages may vary within this range depending upon the dosage form employed and/or the route of administration utilized. the exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects; i.e., the minimal effective concentration (MEC). the MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of agent or composition administered may be dependent on a variety of factors, including the sex, age, and weight of the subject being treated, the severity of the affliction, the manner of administration, and the judgment of the prescribing physician.

A therapeutically effective amount can be the same or different than either one of, or both of, the effective amount of SRA737 and the second effective amount of the further treatment. This is because the present disclosure provides that the methods, as described herein, are effective even where neither the effective amount of SRA737 nor the second effective amount of the further treatment must be an amount that, alone, will ameliorate a symptom of a disease (e.g., the amount of the SRA737 and/or the further treatment may be considered a “sub-therapeutic” amount if administered as an individual therapy). However, the present disclosure does provide that a therapeutically effective amount of the combination must be provided, i.e. the combination does at least affect a treatment of a symptom of a disease.

A unit dose form is a term that is generally understood by the skilled artisan. A unit dose forms is a pharmaceutical drug product that is marketed for a specific use. The drug product includes the active ingredient(s) and any inactive components, most often in the form of pharmaceutically acceptable carriers or excipients. It is understood that multiple unit dose forms are distinct drug products. Accordingly, one unit dose form may be e.g. the combination of SRA737 and a further treatment of 250 mg at a certain ratio of each component, while another completely distinct unit dose form is e.g. the combination of SRA737 and a further treatment of 750 mg at the same certain ratio of each component referred to above. So from one unit dose form to another, the effective amount of SRA737 and the second effective amount of the further treatment may both remain the same. Of course, when the either one of the effective amount of SRA737 or the second effective amount of the further treatment changes, the unit dose form is distinct.

In some aspects, the effective amount is unique to the SRA737 compound. In some aspects, the effective amount of SRA737 is an amount that is equivalent to a “therapeutically effective amount” or an amount that brings about a therapeutic and/or beneficial effect. In some aspects, the effective amount of SRA737 is a “therapeutically effective amount”. In some aspects, the second effective amount of the further treatment is a “therapeutically effective amount”. In some aspects, the third effective amount of the further treatment is a “therapeutically effective amount”. In some aspects, the fourth effective amount of the third treatment is a “therapeutically effective amount”. In some aspects, all of the effective amounts are a “therapeutically effective amount”.

In some aspects, the SRA737 and at least one of more of the further treatment combination is formulated in one (1) unit dose form. In some aspects, the same unit dose form is administered for at least four (4) hours, six (6) hours, eight (8) hours, twelve (12) hours, twenty four (24) hours, one (1) day, two (2) days, three (3) days, seven (7) days, ten (10) days, fourteen (14) days, twenty one (21) days, or thirty (30) days.

In some aspects, the SRA737, the LDG, and an immune checkpoint inhibitor treatment combination is formulated in one (1) unit dose form. In some aspects, the same unit dose form is administered for at least four (4) hours, six (6) hours, eight (8) hours, twelve (12) hours, twenty four (24) hours, one (1) day, two (2) days, three (3) days, seven (7) days, ten (10) days, fourteen (14) days, twenty one (21) days, or thirty (30) days.

In some aspects, the SRA737 and at least one of the further treatment combination is formulated in at least two (2) separately distinct unit dose forms. In some aspects, the first effective amount is different in the first unit dose form than in the second unit dose form. In some aspects, the effective amount of SRA737 is the same in the first unit dose form as it is in the second unit dose form.

In some aspects, the SRA737, the LDG, and an immune checkpoint inhibitor combination is formulated in at least three (3) separately distinct unit dose forms. In some aspects, the first effective amount is different in the first unit dose form than in the second and the third unit dose forms.

In some aspects, the first unit dose form is the same as the second unit dose form. In some aspects, the first unit dose form is the same as the third unit dose form. In some aspects, the first unit dose form is the same as the second and the third unit dose forms. In some aspects, the first unit dose form is the same as the second, third, and fourth unit dose forms.

Compounds of the Invention

In one aspect, the present disclosure provides for methods of use of the compound SRA737.

SRA737

The compound SRA737 is 5-[[4-[[morpholin-2-yl]methylamino]-5-(trifluoromethyl)-2-pyridyl]amino]pyrazine-2-carbonitrile. SRA 737 is disclosed in international patent application no. PCT/GB2013/051233, which is herein incorporated by reference in its entirety. The skilled artisan will find the how to synthesize SRA737 in international patent application no. PCT/GB2013/051233. The structure of SRA737 is

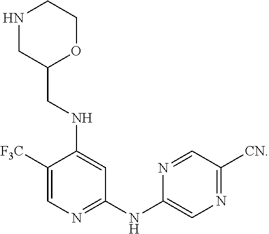 [SMILES: N#Cc1cnc(Nc2cc(NCC3CNCCO3)c(C(F)(F)F)cn2)cn1]

Each of the enantiomers of SRA737 is useful for compositions, methods and kits disclosed herein.

Immune Checkpoint Inhibitor

In one aspect, methods are provided in which SRA737 is administered in combination with at least one checkpoint inhibitor, with or without a further therapeutic agent. As demonstrated in the Examples below, combining SRA737, a Chk1 inhibitor, with low dose gemcitabine and a checkpoint inhibitor provides a synergistic effect on anti-tumor activity.

Illustrative immune checkpoint molecules that can be targeted for blocking or inhibition in the methods described herein include, but are not limited to, CTLA-4, 4-1BB (CD137), 4-1BBL (CD137L), PDL1, PDL2, PD1, B7-H1, B7-H3, B7-H4, BTLA, HVEM, TIM3, GAL9, LAG3, TIM3, B7H3, B7H4, VISTA, KIR, 2B4 (belongs to the CD2 family of molecules and is expressed on all NK, γδ, and memory CD8+(αβ) T cells), CD160 (also referred to as BY55), CGEN-15049, and IDOL. Immune checkpoint inhibitors include antibodies, or antigen binding fragments thereof, or other binding proteins, that bind to and block or inhibit the activity of one or more of CTLA-4, PDL1, PDL2, PD1, B7-H1, B7-H3, B7-H4, BTLA, HVEM, TIM3, GAL9, LAG3, TIM3, B7H3, B7H4, VISTA, KIR, 2B4, CD160, and CGEN-15049 and IDO1. Illustrative immune checkpoint inhibitors useful in the methods described herein include Tremelimumab (CTLA-4 blocking antibody), anti-OX40, PD-L1 monoclonal Antibody (Anti-B7-H1; MEDI4736), MK-3475 (PD-1 blocker), Nivolumamb (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor). Immune checkpoint inhibitors useful in the methods described herein also include small molecule inhibitors that inhibit enzymes involved in immune tolerance pathways, such as inhibitors of IDO1 enzymatic activity. IDO1 inhibitors include, but are not limited to, epacadostat (INCB024360), indoximod (D-1MT), F-001287 (BMS-986205), and NLG919.

In certain embodiments, the immune checkpoint inhibitor is selected from the group consisting of: Tremelimumab (CTLA-4 blocking antibody), anti-OX40, PD-L1 monoclonal Antibody (Anti-B7-H1; MEDI4736), MK-3475 (PD-1 blocker), Nivolumamb (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), Yervoy/ipilimumab (anti-CTLA-4 checkpoint inhibitor), epacadostat (IDO1 inhibitor), indoximod (IDO1 inhibitor), F-001287 (IDO1 inhibitor), and NLG919 (IDO1 inhibitor).

In certain embodiments, the immune checkpoint inhibitor is an anti-CTLA-4 antibody. In certain embodiments, the immune checkpoint inhibitor is an anti-PD-L1 antibody. In certain embodiments, the immune checkpoint inhibitor is an anti-PD1 antibody. In certain embodiments, the immune checkpoint inhibitor is an IDO1 inhibitor. In some embodiments, the immune checkpoint inhibitor is a combination of two or more immune checkpoint inhibitors selected from an anti-CTLA-4 antibody, an anti-PD-L1 antibody, an anti-PD1 antibody, and an IDO1 inhibitor. In a particular embodiment, the immune checkpoint inhibitor is an IDO1 inhibitor in combination with an anti-CTLA-4 antibody, an anti-PD-L1 antibody, an anti-PD1 antibody, and an IDO1 inhibitor.

Combination Therapies

In another aspect, the present disclosure provides for methods of use of the compound SRA737 in a combination therapy with at least one or more other treatments.

Further treatments include, but are not limited to, administering a chemotherapeutic agent, administering an antibody or antibody fragment (such as an immune checkpoint inhibitor), administering a radiation treatment, administering an external inducer of replication stress, and administering a combination thereof.

The term “chemotherapy” refers to administration of any genotoxic agent (e.g., DNA damaging agent), including conventional or non-conventional chemotherapeutic agents, for the treatment or prevention of cancer. Chemotherapeutic agents include agents that have been modified, (e.g., fused to antibodies or other targeting agents). Examples of chemotherapeutic agents include, but are not limited to, platinum compounds (e.g., cisplatin, carboplatin, oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, bendamustine, mitomycin C), antitumor antibiotics (e.g., daunorubicin, doxorubicin, idarubicin, epirubicin, mitoxantrone, bleomycin, plicamycin, dactinomycin), taxanes (e.g., paclitaxel, nab-paclitaxel and docetaxel), antimetabolites (e.g., 5-fluorouracil, cytarabine, premetrexed, thioguanine, floxuridine, capecitabine, and methotrexate ), nucleoside analogues (e.g., fludarabine, clofarabine, cladribine, pentostatin, nelarabine, gemcitabine, 5-flurouracil), topoisomerase inhibitors (e.g., topotecan, irinotecan, SN-38, CPT-11), hypomethylating agents (e.g., azacitidine and decitabine ), proteasome inhibitors (e.g., bortezomib ), epipodophyllotoxins (e.g., etoposide and teniposide ), DNA synthesis inhibitors (e.g., hydroxyurea), and vinca alkaloids (e.g., vincristine, vindesine, vinorelbine, and vinblastine ). Chemotherapeutic agents include DNA intercalating agents (e.g., pyrrolobenzodiazepines).

The term “external inducer of replication stress” refers to any agent that causes increased stalled replication forks, increased genomic instability, increased mutation and/or mutation rate, activation of DNA damage repair pathways, activation of the DNA damage response (DDR), activation or increased expression of replication stress gene(s), or combinations thereof. Examples of inducers of replication stress include, but are not limited to, genotoxic chemotherapeutic agents (e.g., gemcitabine and other nucleoside analogs, alkylating agents such as temozolomide, cisplatin, mitomycin C and others, topoisomerase inhibitors such as camptothecin and etoposide and others). External inducers of cell stress include agents that reduce the concentration of nucleotides in a cell (e.g., ribonucleotide reductase inhibitors and the like). External inducers of cell stress include agents also include PARP inhibitors.

The term “DNA damage repair (DDR) gene” or “DNA damage repair pathway gene” refers to any gene that directly or indirectly promotes repair of DNA mutations, breaks or other DNA damage or structural changes. DNA damage repair genes include, but are not limited to, the following genes: ATM, CDK12, RAD51C. BRCA1, BRCA2, MRE11A, ATR, and Rad50. DDR genes also include genes in the Fanconi anemia (FA) pathway. Genes in the FA pathway include, but are not limited to, Fanconi anemia complementation group (FANC) genes.

The term “immune checkpoint inhibitor” refers to binding molecules that bind to and block or inhibit the activity of one or more immune checkpoint molecules or drugs that inhibit immunosuppressive proteins. Illustrative immune checkpoints inhibitors include antibodies, or antigen binding fragments thereof, that target one or more of CTLA-4, PD-L1, PD-L2, PD1, B7-H3, B7-H4, BTLA, HVEM, TIM3, GAL9, LAG3, TIM3, B7H3, B7H4, VISTA, KIR, 2B4, CD160, CGEN-15049, and IDO1.

The term “PARP inhibitor” or “PARPi” refers to an inhibitor of PARP. A PARPi may be a small molecule, an antibody or a nucleic acid. A PARPi may function to reduce the expression of PARP or the activity of PARP in cells, or combinations thereof. PARPi include inhibitors that do or do not alter the binding of PARP to DNA. PARPi may inhibit any members of the PARP family. PARPi include, but are not limited to: Olaparib, Rucaparib, Veliparib, Niraparib, Iniparib, Talazoparib, Veliparib, Fluzoparib, BGB-290, CEP-9722, BSI-201, EZ449, PF-01367338, AZD2281, INO-1001, MK-4827, SC10914, and 3-aminobenzamine.

In specific aspects, further treatments include, but are not limited to, administering any one of gemcitabine, olaparib, niraparib, rucaparib, talazoparib, cisplatin, a ribonucleotide reductase inhibitor, etoposide, SN-38/CPT-11, mitomycin C, and combinations thereof.

Pharmaceutical Compositions of the Invention

Methods for inhibiting the growth of a tumor, inhibiting the progression of or treating cancer are described herein. Said methods of the invention include administering an effective amount of SRA737 and a second effective amount of a further treatment. the SRA737 and the further treatment can each be formulated in pharmaceutical compositions. these pharmaceutical compositions may comprise, in addition to the active compound(s), a pharmaceutically acceptable excipient, carrier, buffer, stabilizer or other materials well known to those skilled in the art. Such materials should be non-toxic and should not interfere with the efficacy of the active ingredient. the precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin. Liquid pharmaceutical compositions generally include a liquid carrier such as water or oil, including oils of petroleum, animal, vegetable or synthetic origin, e.g., peanut oil, soybean oil, mineral oil, sesame oil, etc. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

A composition can be administered alone or in combination with other treatments, either simultaneously or sequentially dependent upon the condition to be treated.

The present technology is not limited to any particular composition or pharmaceutical carrier, as such may vary. In general, compounds of the present technology will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of the present technology is inhalation.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. there are several types of pharmaceutical inhalation devices-nebulizer inhalers, metered dose inhalers (MDI) and dry powder inhalers (DPI). Nebulizer devices produce a stream of high velocity air that causes therapeutic agents (which are formulated in a liquid form) to spray as a mist that is carried into the subject's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the subject's inspiratory air-stream during breathing by the device. In order to achieve a free flowing powder, therapeutic agent is formulated with an excipient such as lactose. A measured amount of therapeutic agent is stored in a capsule form and is dispensed with each actuation.

Pharmaceutical dosage forms of a compound of the present technology may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present technology can include one or more physiologically acceptable inactive ingredients that facilitate processing of active molecules into preparations for pharmaceutical use.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the present technology in combination with at least one pharmaceutically acceptable excipient. Acceptable excipients are non-toxic, aid administration, and do not adversely affect therapeutic benefit of the claimed compounds. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including oils of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of the present technology in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

In some embodiments, the pharmaceutical compositions include a pharmaceutically acceptable salt. The term “pharmaceutically acceptable salt” refers to salts derived from a variety of organic and inorganic counter ions well known in the art that include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in Stahl and Wermuth (Eds.), Handbook of Pharmaceutical Salts Properties, Selection, and Use; 2002.

The present compositions may, if desired, be presented in a pack or dispenser device containing one or more unit dosage forms containing the active ingredient. Such a pack or device may, for example, comprise metal or plastic foil, such as a blister pack, or glass, and rubber stoppers such as in vials. the pack or dispenser device may be accompanied by instructions for administration. Compositions comprising a compound of the present technology formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01-99.99 wt % of a compound of the present technology based on the total formulation, with the balance being one or more suitable pharmaceutical excipients. Preferably, the compound is present at a level of about 1-80 wt %. Representative pharmaceutical formulations are described below.

### Formulation Examples

The following are representative pharmaceutical formulations containing the SRA737 and a further treatment, either alone or in combination.

A composition can be administered alone or in combination with other treatments, either simultaneously or sequentially dependent upon the condition to be treated.

Kits

The present disclosure also provides for a kit comprising the combination of SRA737 and at least one further treatment and instructions for use. The present disclosure further provides for a kit comprising one or more pharmaceutical compositions where the pharmaceutical composition(s) comprise SRA737 and at least one further treatment, and instructions for use, optionally the combination includes at least one pharmaceutically acceptable carrier or excipient.

Individual components of the kit can be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the antigen-binding construct.

In some aspects, the disclosure provides for a kit comprising a combination of SRA737 and at least one further treatment and at least one pharmaceutically acceptable carrier or excipient.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilized form and the kit can additionally contain a suitable solvent for reconstitution of the lyophilized components. Irrespective of the number or type of containers, the kits described herein also may comprise an instrument for assisting with the administration of the composition to a patient. Such an instrument may be an inhalant, nasal spray device, syringe, pipette, forceps, measured spoon, eye dropper or similar medically approved delivery vehicle.

In another aspect described herein, an article of manufacture containing materials useful for the treatment, prevention and/or diagnosis of the disorders described herein, e.g., inhibition of tumor growth is provided. The article of manufacture comprises a container and a label or package insert on or associated with the container. Suitable containers include, for example, bottles, vials, syringes, iv. solution bags, etc. The containers may be formed from a variety of materials such as glass or plastic. The container(s) holds a composition which is by itself or combined with another composition effective for treating, preventing and/or diagnosing the disorder and may have a sterile access port (for example the container may be an intravenous solution bag or a vial having a stopper pierceable by a hypodermic injection needle).

The article of manufacture in this embodiment described herein may further comprise a label or package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Polypeptides and Nucleic Acids

Described herein are polypeptide and nucleic acid sequences of genes useful for the invention, e.g., genes for Chk1. In some embodiments, polypeptide and nucleic acid sequences useful for the invention are at least 95, 96, 97, 98, or 99% identical to sequences described herein or referred to herein by a database accession number. In some embodiments, polypeptide and nucleic acid sequences useful for the invention are 100% identical to sequences described herein or referred to herein by a database accession number.

The term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra). One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

### EXAMPLES

Below are examples of specific embodiments for carrying out the present invention. the examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning. A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Abbreviations

TABLE 1B
Abbreviations

| | |
|---|---|
| QD | Once a day |
| BID | Twice a day |
| BIW | Twice weekly |
| p.o. | Oral(ly) |
| i.p. | Intraperitoneal (ly) |
| RT | Room Temperature |
| BW | Body weight |
| BWL | Body weight loss |
| FFPE | Formalin Fixed Paraffin Embedded |
| SD | Standard deviation |
| SEM | Standard error of mean |
| AE | Adverse event |
| MTD | Maximum tolerated dose |
| HNSTD | Highest non-severely toxic dose |
| NOAEL | No-observed-adverse-effect-level |

### Example 1: Summary of Nonclinical Pharmacology

SRA737 was previously found to be a potent and selective inhibitor of Chk1 with limited off-target activity against other kinases, for example, as described in more detail in Walton et al. (Oncotarget. 2016 Jan. 19; 7(3): 2329-2342), herein incorporated by reference for all it teaches. In vitro, SRA737 potently inhibited genotoxic chemotherapy-induced Chk1 autophosphorylation and prevented downstream signal transduction (data not shown). This Chk1 inhibition produced the expected dose-dependent inhibition of genotoxicity-induced checkpoint arrest and a SRA737 dose-dependent potentiation of the cytotoxicity of genotoxic chemotherapeutic agents and targeted agents.

A program of in vivo efficacy studies was performed to assess the activity of SRA737 in combination with genotoxic chemotherapy and targeted agents, and as monotherapy.

Significant, dose-dependent antitumor activity of SRA737 in combination with standard-dose gemcitabine was noted in multiple cancer xenograft models including HT29 human colon cancer, SJSA-1 human osteosarcoma (with LDG treatment only), SW620 mouse colon cancer, Calu6 human (NSCLC), KPC-1 pancreatic cancer and patient-derived bladder carcinoma (data not shown). Synergy was also observed with low-dose gemcitabine in the HT29, OVCAR3 and SJSA-1 CDX models as well as in a PDX model of TNBC; with gemcitabine and carboplatin in the Calu6 model, and with irinotecan in the HT29 model (data not shown). Significant antitumor activity was also observed in multiple syngeneic mouse models (mTmG, MC38, MBT2 and Pan02) in combination with a PD1/PD-L1 inhibitor (data not shown). Significant antitumor activity of SRA737 presented as a single agent was observed in several HGSOC PDX models harboring CCNE1 amplifications accompanied by TP53 mutations (data not shown). One of these models also carried a MYCN amplification. A fourth HGSOC PDX model with partial resistance to PARPi was also sensitive to high dose SRA737 monotherapy. SRA737 also demonstrated single agent efficacy in the OVCAR3 model of HGSOC, Ep-Myc model of B-cell lymphoma; MOLM-13 model of AML; TH-MYC model of neuroblastoma; MDA-MB-231 model of TNBC and in two syngeneic models of renal and lung cancers (Renca and LL/2, respectively) (data not shown).

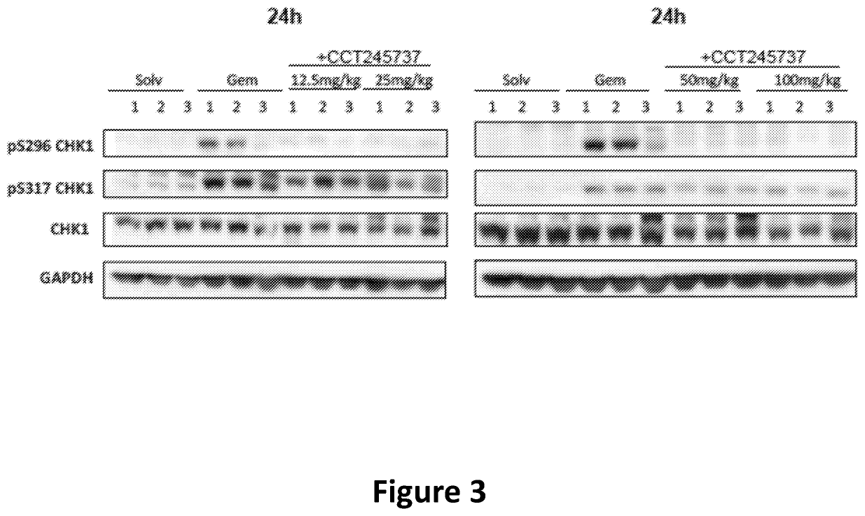

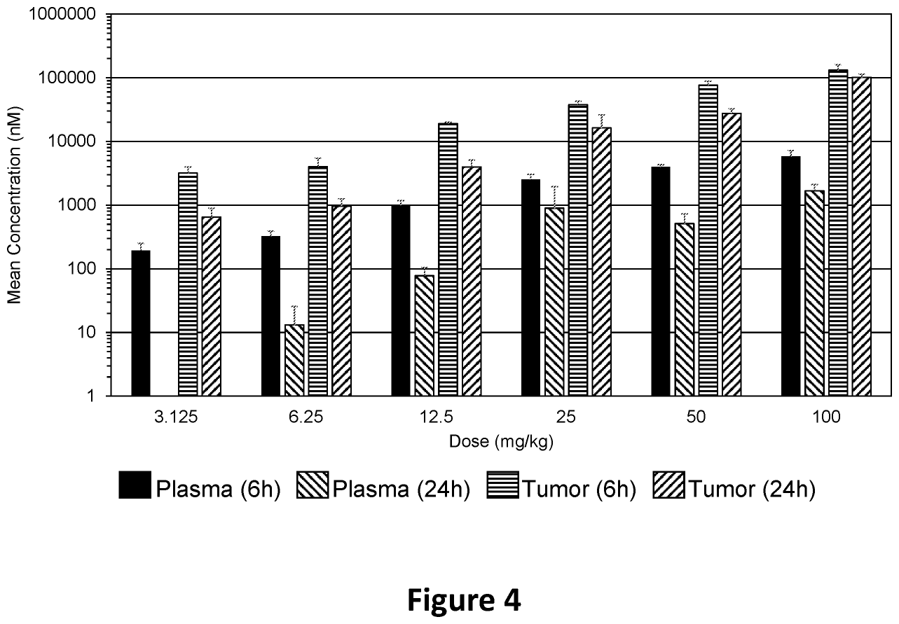

The effect of SRA737 on Gemcitabine induced Chk1 S296 autophosphorylation was assessed as described in Walton et al. (Oncotarget. 2016 Jan. 19; 7(3): 2329-2342), herein incorporated by reference for all it teaches. Briefly, mice bearing HT29 tumor xenografts were administered (i) vehicle control or (ii) gemcitabine (100 mg/kg in saline, IV) or (iii) a combination of SRA737 (12.5, 25, 50 or 100 mg/kg in DTPW, oral) and gemcitabine (100 mg/kg) with SRA737 administered 24 hours following gemcitabine administration (n=3 per time point per treatment). Inhibition of pS296 Chk1 was observed at SRA737 doses greater than or equal to 12.5 mg/kg (FIG. 3), which corresponded to a minimum (total) plasma concentration of approximately 100 nM (actual value 78±27 nM, ˜40 ng/mL) at 24 hours (FIG. 4 and Table 2). Exposure in the tumor was greater than 10-fold higher than in plasma. The circulating plasma concentration at this 24-hour timepoint corresponded to a free drug concentration of approximately 6 nM (2 ng/mL) based on plasma protein binding in mice of 94%.

The PK/PD data showed that relatively low, plasma concentrations of SRA737 sustained above an effective concentration (e.g., SRA737 exceeding a 100 nM plasma concentration for 24 hours) elicited significant antitumor activity in mice and provides a PK/PD benchmark for application in a clinical setting.

TABLE 2
SRA737 plasma concentrations and tumor concentrations in a HT29 xenograft model

| Dose | | | | | | | | |
|---|---|---|---|---|---|---|---|---|
| (mg/kg) | Plasma (6 h) | | Plasma (24 h) | | Tumor (6 h) | | Tumor (24 h) | |
| 3.125 | 193.9256 | 55.89237 | 0 | 0 | 3183.276 | 787.7809 | 645.7623 | 239.2542 |
| 6.25 | 323.3166 | 64.99931 | 13.10728 | 12.62854 | 4011.501 | 1421.794 | 965.7797 | 283.3167 |
| 12.5 | 1007 | 165.5266 | 77.7 | 26.58816 | 19009.57 | 1066.455 | 3974.933 | 1099.057 |
| 25 | 2517.067 | 532.9315 | 893.4667 | 1048.18 | 37516 | 5539.576 | 16289.53 | 9893.223 |
| 50 | 3957.5 | 339.5922 | 512.1 | 215.3084 | 76331.2 | 11510.94 | 27483.73 | 4915.968 |
| 100 | 5837.767 | 1365.599 | 1674.5 | 431.4472 | 131506.7 | 28331.53 | 101764.1 | 11743.44 |

### Example 2: Summary of Pharmacokinetic Studies

Several studies have been conducted to evaluate the PK properties of SRA737, such as the absorption (in vitro permeability assays and in vivo PK following IV and oral administration), distribution (in vivo tissue distribution and in vitro plasma protein binding) and metabolism (in vitro hepatocyte and CYP inhibition and induction studies) of SRA737.

The PK of SRA737 have been determined in the mouse, rat, dog and monkey following oral and IV administration (Table 3). Very favorable absolute oral bioavailability (% F) was noted, particularly in the mouse (105%) and monkey (90-104%), consistent with the moderate metabolism and favorable permeability noted in in vitro models. An acceptable terminal elimination t_{1/2} was also observed in each species. In addition, the effect of prandial state on the PK of the SRA737 clinical drug product capsule presentation was evaluated in dogs. There was no significant effect of prandial state on oral bioavailability (Table 4). The plasma protein binding of SRA737 at 1 and 10 μM was examined in mouse, minipig, monkey and human plasma using ultracentrifugation and in dog plasma (10 μM) using rapid equilibrium dialysis. Moderate plasma protein binding was observed in humans (˜87%) and the non-rodent toxicology species (˜80% and 87% for the minipig and monkey, respectively), whereas high plasma protein binding (˜94%) was observed in the mouse (Table 5).

TABLE 3
Summary of PK Parameters of 5RA737 After a Single Dose

| Species | Dose | Cₘₐₓ | Tₘₐₓ | AUCₗₐₛₜ | t_{1/2} | Fₒᵣₐₗ |
|---|---|---|---|---|---|---|
| and route | (mg/kg) | (μg/mL) | (h) | (μg · h/mL) | (h) | (%) |
| Mouse IV | 10 | — | — | 3.77 | 2.9 | — |
| Mouse oral | 10 | 0.61 | 1 | 3.94 | 2.8 | 105 |
| Rat IV | 5 | — | — | 1.38 | 1.0 | — |
| Rat oral | 10 | 0.19 | 2 | 1.17 | 4.6 | 42 |
| Dog IV | 1 | — | — | 0.20 | 2.8 | — |
| Dog oral | 5 | 0.13 | 1-3 | 0.85 | 3.5 | 86 |
| Monkey IV | 1 | — | — | 1.52 | 5.3 | — |
| Monkey oral | 2 | 0.50 | 1-2 | 2.73 | 5.0 | 90 |
| Monkey oral | 10 | 2.64 | 2-4 | 15.84 | 5.1 | 104 |

TABLE 4
Summary of PK Parameters of SRA737 Drug Product After a
Single Dose in Fasted and Non-Fasted Dogs

| Route/Prandial | | | Cₘₐₓ | Tₘₐₓ | AUCₗₐₛₜ | t_{1/2} | Fₒᵣₐₗ |
|---|---|---|---|---|---|---|---|
| State | Dose | | (μg/mL) | (h) | (μg · h/mL) | (h) | (%) |
| IV (Fasted^{a}) | 1 | mg/kg | — | — | 0.587 | 2.2 | — |
| IV (Non-fasted^{b}) | 1 | mg/kg | — | — | 0.518 | 1.5 | — |
| Oral (Fasted^{a}) | 100 | mg/dog | 0.75 | 1.5 | 4.29 | 2.6 | 79 |
| Oral (Non-fasted^{b}) | 100 | mg/dog | 0.64 | 2.0 | 3.28 | 2.2 | 65 |

^{a}Fasted animals fasted overnight and only fed at 4 h postdose
^{b}Non-fasted animals fasted overnight and fed 1 h predose

TABLE 5
Summary of the Percentage Plasma Protein Binding and
Percentage Free 5RA737 in Mouse, Minipig, Human, Dog and
Monkey

| | Percentage Bound | | Percentage Free | |
|---|---|---|---|---|
| Species | 1 μM | 10 μM | 1 μM | 10 μM |
| Mouse | 94.5 | 93.4 | 5.5 | 6.6 |
| Minipig | 85.2 | 75.5 | 14.9 | 24.5 |
| Human | 87.7 | 87.2 | 12.4 | 12.8 |
| Dog | N/A | 68.8 | N/A | 31.2 |
| Monkey | 88.8 | 85.8 | 11.2 | 14.2 |

The membrane permeability of SRA737 was assessed in the parallel artificial membrane permeability assay (PAMPA) and Caco-2 assays. Permeability in the PAMPA assay was classified as low. At 10 μM permeability in the Caco-2 assay was 20.7±9.1×10⁻⁶ cm/s with an efflux ratio (A>B/B>A) of 0.8, which indicated that SRA737 has a relatively high passive permeability and low efflux potential.

The formation of SRA737-related metabolites was determined in cryopreserved hepatocytes from human, mouse, rat, dog, minipig and monkey samples after incubation with SRA737 at a nominal concentration of 10 μM for up to 4 hours. The rank order of stability from most stable to least stable for the species was rat≈mouse >monkey≈human >>dog >>minipig. In the human hepatocyte preparation, approximately 67% of the parent remained after 4 hours of incubation compared to 75% and 7% in the rat and minipig preparations, respectively. Eight human SRA737 metabolites were observed. All SRA737 metabolites formed by human hepatocytes were also formed by monkey hepatocytes. Six metabolites were present at equal or greater abundance in the monkey. No human-specific metabolites were observed, but two of the human metabolites were not formed in any other species at equal or greater abundance.

The excretion of SRA737 has been studied in mice and rats administered SRA737 at either 5 mg/kg IV or 10 mg/kg orally. Urine and feces were collected for a 24-hour period after dosing. In mice, renal excretion of intact SRA737 was consistently low (less than 10% of dose) after both oral and IV administration. Following IV administration of SRA737, less than 8% of the dose was recovered as intact drug in the feces while after oral administration this figure was less than 3%. Excretion of intact SRA737 was lower in the rat than in the mouse with <1% of dose excreted renally over 24 hours and less than 1.5% of dose excreted into the feces over 24 hours.

No significant inhibition of major CYP enzymes (1A2, 2A6, 2C9, 2C19, 2D6 and 3A4) was observed at the highest concentration of SRA737 investigated (IC₅₀ >10-50 μM), suggesting that the compound is unlikely to mediate significant metabolic drug-drug interactions. Minimal, concentration dependent induction of CYP1A2 (<10% positive control, omeprazole) was observed in vitro suggesting SRA737 in some cases minimally affected the metabolism of concomitantly administered drugs that are predominately metabolized by CYP1A2.

Taken together, the permeability, metabolic stability, and demonstrable oral bioavailability observed in preclinical species is suggestive of favorable oral absorption in humans.

### Example 3: Summary of Toxicology Findings

The toxicity of SRA737 was assessed in Good Laboratory Practice (GLP) 28-day repeat oral dose studies in the mouse, minipig and monkey and in a three-cycle combination study with gemcitabine and cisplatin in the mouse.

Toxicokinetic data for SRA737 administered as a monotherapy are summarized in Error! Reference source not found.6. The pattern of toxicology findings observed in the pivotal studies in mouse, minipig and monkey were broadly similar and consistent with SRA737's mechanism of action although in general, the monkey appeared to be the least sensitive toxicological species. Data from studies in the monkey suggest higher exposures would likely be tolerated in humans than would be predicted from mouse and minipig data. Based on similarities between monkey and human data for plasma protein binding, stability in hepatocytes, and other ADMET data, the monkey has been confirmed as the most suitable nonclinical model for the determination of potential human toxicity.

Dose-dependent toxicological findings related to bone marrow toxicity, including variously decreased red and white cell parameters with increased medullary or extramedullary hematopoiesis and atrophy of lymphatic organs including the thymus was noted in the mouse and minipig pivotal studies. These findings were reversible on cessation of drug administration. Toxicological findings in the GI tract were also observed in the minipig and in early mouse and monkey studies and changes in reproductive organs, particularly the testes, were also observed in the minipig and mouse, but not monkey. these latter changes were not reversible in the mouse; however, the relevance of these findings in sexually immature animals to adult cancer patients appears limited.

The MTD was 75 mg/kg/day (225 mg/m²/day) in the mouse and the HNSTD was 10 mg/kg/day (350 mg/m²/day) in the minipig. An absence of toxicological findings was noted in the pivotal monkey toxicity study, thus the NOAEL of 20 mg/kg/day (240 mg/m²/day) was the highest dose tested.

Findings from a triple combination study in mice (SRA737 administered in combination with IV gemcitabine and cisplatin on an intermittent schedule over 18 days) mirrored those noted in the monotherapy toxicity study in this species, although reversible intestinal epithelial degeneration was also noted in the high dose triplet combination group. Only the reversible marrow toxicity, consequent splenomegaly, and the high-dose intestinal observations were deemed to have been exacerbated by administration of SRA737 over those observed following administration of the cisplatin/gemcitabine control group alone. Clinical combination of SRA737 and gemcitabine in some cases therefore elicited exacerbated hematological and GI toxicities.

TABLE 6
Summary of the Toxicokinetic Data on 5RA737 Administered as a Monotherapy
(Daily Dosing of SRA737 for 28 Days)

| | Dose | Mouse | | Minipig | | Monkey | |
|---|---|---|---|---|---|---|---|
| Parameter | (mg/kg/day) | Male | Female | Male | Female | Male | Female |
| Cmax | 2 | — | — | — | — | 0.365 | 0.519 |
| (μg/mL) | 10 | 0.903 | 0.918 | 0.622* | 0.515* | 2.07 | 1.87 |
| | 20 | — | — | — | — | 3.17* | 2.32* |
| | 40 | 3.25 | 2.35 | 2.07 | 2.83 | — | — |
| | 75 | 5.45* | 4.03* | 5.34 | — | — | |
| AUC_{0 - t} | 2 | — | — | — | — | 3.24 | 2.58 |
| (μg · h/mL) | 10 | 4.41 | 3.61 | 6.48* | 6.56* | 17.9 | 12.9 |
| | 20 | — | — | — | — | 20.7* | 24.2* |
| | 40 | 34.3 | 24.8 | 22.21 | 45.48 | — | — |
| | 75 | 68.6* | 45.5* | — | 97.02 | — | — |
| T_{1/2} | 2 | — | — | — | — | 6.73 | 8.74 |
| (hours) | 10 | — | 5.18 | 9.25* | 13.6* | 8.34 | 6.58 |
| | 20 | — | — | — | — | 6.84* | 6.00* |
| | 40 | 6.22 | 5.09 | 7.66 | 14.3 | — | — |
| | 75 | 8.36* | 6.96* | — | 31.5 | — | — |

*data obtained at the MTD, HNSTD and NOAEL for mouse, minipig and monkey, respectively

### Example 4: Phase 1 Clinical Study to Establish the Maximum Tolerated Dose and Blood Plasma Concentration in a SRA737 Monotherapy

A Phase 1 clinical trial was conducted in ‘all comers,’ i.e. no genetic selection was performed, to establish safety, tolerability and pharmacokinetics (“Dose Escalation Phase”). Cohorts consisting initially of a single subject received escalating doses of SRA737, starting in Cohort 1 with 20 mg/day administered orally on a continuous daily dosing schedule in 28 day cycles. the dose was escalated until the maximum tolerated dose (MTD) was identified.

In the Dose Escalation Phase, 18 subjects received SRA737 in 9 dose level cohorts, from 20 to 1300 mg QD; median treatment duration 62.5 days (range 1 to 226). Dose level cohorts of up to 1000 mg of SRA737 were completed without any dose-limiting toxicities (DLTs). Two of 3 subjects experienced DLTs at the 1300 mg once daily dose, each being an inability to receive 75% of the planned SRA737 dose due to GI intolerability, with the individual GI effects being low grade. Hence, 1300 mg exceeded the maximum tolerated dose with the once daily dosing regimen. A cohort receiving 500 mg twice daily was added to determine if a twice daily dosing schedule can improve GI tolerability, given the half-life of SRA737 is approximately 10 hours. One of 6 subjects experienced DLTs in the 500 mg twice daily cohort; this was an inability to receive 75% of the planned SRA737 dose due to grade 4 thrombocytopenia with grade 3 neutropenia and anemia. Based on overall tolerability and GI events (nausea, vomiting, and diarrhea), subjects were also enrolled at a dose level of 800 mg once daily and was overall better tolerated than 1000 mg (subjects required fewer dose reductions, experienced fewer severe (G3/4) AEs and significantly less fatigue AEs. The maximum tolerated dose (MTD) was established at 1000 mg QD or 500 mg BID.

Pharmacokinetic parameters for the monotherapy cohorts were monitored and are summarized in Table 7. The Cₘₐₓ and AUC₀₋₂₄ at 1000 mg QD were 2391 ng/mL and 26795 ng·h/mL respectively. Cₘᵢₙ was calculated at 1000 mg QD (411 ng/mL) and exceeded that determined in preclinical models to be effective. Doses ≥300 mg QD also exceeded the preclinical models to be effective.

TABLE 7
SRA737 Monotherapy Mean (±SD) Pharmacokinetic Parameters

| SRA737 | Day −7^{a} | | | | Day 15/22^{b} | | | |
|---|---|---|---|---|---|---|---|---|
| Dose | Tₘₐₓ | Cₘₐₓ | AUC₀₋₂₄ | T_{1/2} | Tₘₐₓ | Cₘₐₓ | AUC₀₋₂₄ | T_{1/2} |
| (mg) | (h) | (ng/mL) | (ng · h/mL) | (h) | (h) | (ng/mL) | (ng · h/mL) | (h) |
| 20 | 1.0 | 43.0 | 130 | — | 4.0 | 29.0 | 342 | 17.6 |
| | (−) | (−) | (−) | | (−) | (−) | (−) | (−) |
| 40 | 1.0 | 95.0 | 809 | 9.85 | 2.0 | 75.9 | 1,006 | 16.8 |
| | (−) | (−) | (−) | (−) | (−) | (−) | (−) | (−) |
| 80 | 4.0 | 68.9 | 691 | 9.27 | 2.0 | 81.4 | 1,064 | 15.6 |
| | (−) | (−) | (−) | (−) | (−) | (−) | (−) | (−) |
| 160 | 4.0 | 178 | 1,618 | 13.8 | 2.0 | 217 | 2,515 | 19.8 |
| | (−) | (−) | (−) | (−) | (−) | (−) | (−) | (−) |
| 300 | 4.0 | 353 | 3,152 | 8.59 | 6.0 | 145 | 2,362 | 15.4 |
| | (−) | (−) | (−) | (−) | (−) | (−) | (−) | (−) |
| 600 | 1.8 | 998 | 8,870 | 12.9 | 2.9 | 1,381 | 12,850 | 10.4 |
| | (0.5) | (337) | (3,170) | (1.35) | (1.1) | (819) | (5,943) | (1.92) |
| 800 | 3.3 | 1,980 | 18,856 | 10.3 | 2.5 | 2,573 | 25,423 | 10.0 |
| | (1.2) | (633) | (6,211) | (1.34) | (0.9) | (690) | (8,693) | (4.71) |
| 1000 | 3.3 | 2,099 | 20,283 | 11.0 | 3.1 | 2,331 | 25,991 | 9.38 |
| | (1.7) | (814) | (7,744) | (1.72) | (1.8) | (687) | (8,549) | (2.20) |
| 1300 | 2.7 | 3,228 | 29,796 | 11.8 | — | — | — | — |
| | (1.2) | (1,320) | (11,233) | (3.11) | | | | |

^{a}20, 40, 80, 160, 300 mg − n = 1; 600 mg − n = 4; 800 mg − n = 25; 1000 mg − n = 43 − 44; and 1300 mg − n = 3
^{b}20, 40, 80, 160, 300 mg − n = 1; 600 mg − n = 7; 800 mg − n = 12; 1000 mg − n = 17

### Example 5: Phase 1 Clinical Study to Establish the Maximum Tolerated Dose and Blood Plasma Concentration in a SRA737 Combination Therapy

A Phase 1 clinical trial was conducted in ‘all comers,’ i.e. no genetic selection was performed, to establish safety, tolerability and pharmacokinetics for SRA737 administered in combination with gemcitabine (“Dose Escalation Phase”). Cohorts consisting initially of a single subject received escalating doses of SRA737, starting in Cohort 1 with 40 mg/day administered orally on days 2, 3, 9, 10, 16, and 17 of each 28-day cycle. Cohorts also received various doses of gemcitabine, starting in Cohort 1 with 300 mg/m²/day administered IV over 30 minutes on days 1, 8, and 15 of each 28-day cycle.

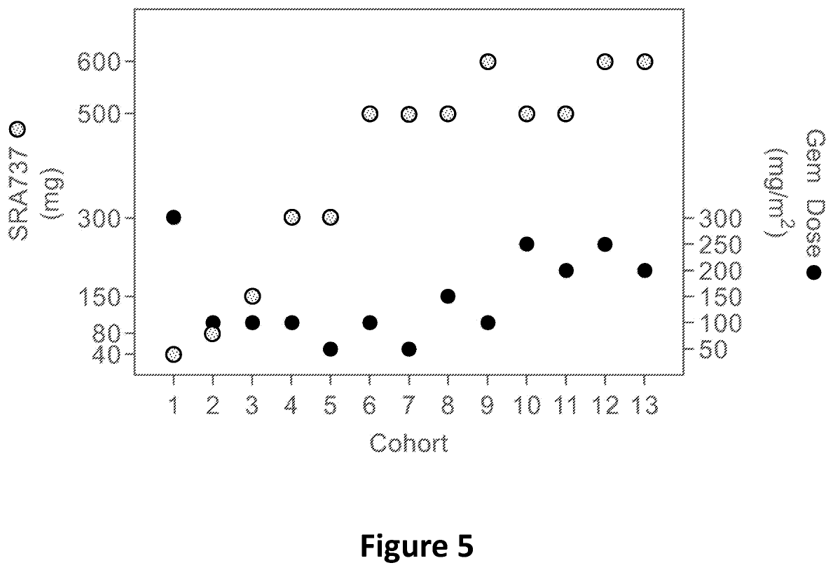

FIG. 5 presents a summary of dosing amounts for the cohorts tested. In the Dose Escalation Phase, a total of 55 subjects received SRA737 in 13 dose escalation cohorts at doses of 40 to 600 mg SRA737 combined with LDG doses of 50 to 300 mg/m². No protocol-defined dose limiting toxicities (DLTs) have been observed.

Pharmacokinetic parameters for the monotherapy cohorts were monitored and are summarized in Table 8. The pharmacokinetic profile of SRA737 revealed AUC₀₋₂₄ and Cₘₐₓ of 3550 ng·h/mL and 548 ng/mL at 150 mg SRA737. At this dose, the Cₘᵢₙ (52 ng/mL) exceeded that determined in preclinical models to be effective.

TABLE 8
SRA737 Combination Therapy Mean (±SD) Pharmacokinetic Parameters

| SRA737 | Day −7^{a} | | | | Day 10^{b} | | | |
|---|---|---|---|---|---|---|---|---|
| Dose | Tₘₐₓ | Cₘₐₓ | AUC₀₋₁₂ | AUC₀₋₂₄ | T_{1/2} | Tₘₐₓ | Cₘₐₓ | AUC₀₋₁₂ |
| (mg) | (h) | (ng/mL) | (ng · h/mL) | (ng · h/mL) | (h) | (h) | (ng/mL) | (ng · h/mL) |
| 20 | 1.9 | 31.3 | 145 | — | 4.09 | — | — | — |
| | (0.9) | (17.3) | (67.9) | | (1.31) | | | |
| 40 | 2.0 | 106 | 486 | 637 | 11.6 | 1.8 | 117 | 543 |
| | (0.0) | (35.9) | (116) | (140) | (2.95) | (0.5) | (32.0) | (160) |
| 80 | 2.0 | 146 | 641 | 825 | 11.3 | 2.0 | 113 | 570 |
| | (0.0) | (28.6) | (143) | (176) | (0.75) | (0.0) | (26.7) | (100) |
| 150 | 1.8 | 548 | 2,630 | 3.550 | 12.7 | 1.5 | 331 | 1,677 |
| | (0.5) | (63.9) | (944) | (1,516) | (1.13) | (0.9) | (307) | (1,439) |
| 300 | 2.4 | 995 | 4,534 | 5,907 | 11.8 | 1.7 | 1,079 | 5,140 |
| | (1.6) | (449) | (1,593) | (1,882) | (0.99) | (1.1) | (564) | (1,606) |
| 500 | 2.4 | 1,433 | 8,318 | 11,417 | 11.8 | 2.4 | 1,723 | 10,191 |
| | (1.4) | (729) | (4,603) | (6,678) | (2.01) | (2.6) | (865) | (5,772) |
| 600 | 1.8 | 2,208 | 11,805 | 15,072 | 11.1 | 2.5 | 2,109 | 11,860 |
| | (0.5) | (359) | (2,477) | (3,041) | (1.31) | (1.0) | (536) | (3,241) |

^{a}20 mg − n = 8 − 10; 40 mg − n = 6; 80 mg − n = 3; 150 mg − n = 4; 300 mg − n = 7; 500 mg − n = 29; and 600 mg − n = 4
^{b}40 mg − n = 4; 80, 150 mg − n = 3; 300 mg − n = 7; 500 mg − n = 19 − 22; and 600 mg − n = 4

Additional cohorts are monitored escalating the dose of SRA737 until the maximum tolerated dose (MTD) is identified and to optimize combination dosing with gemcitabine. All enrolled subjects who receive at least 1 dose of SRA737 and provide at least 1 evaluable PK concentration or have evaluable data for each specific PDn assessment are evaluable for PK and PDn, respectively. Serious adverse events (SAEs) are collected starting on the date of informed consent. Radiological assessment are performed within 4 weeks from the first dose of SRA737 (or gemcitabine if the SRA737 dose for PK is omitted) and repeated every 6 weeks in Stage 1. In Stage 2, assessments are performed every 8 weeks and in long-term follow-up every 16 weeks. Assessments are performed more frequently, when clinically indicated. Cardiac assessments (echocardiogram [ECHO] and electrocardiogram [ECG]) are be conducted. Optional triplet tumor biopsies in some cases are collected within 28 days prior to receiving the first SRA737 dose. Within 7 days of the first dose of SRA737 (or gemcitabine if the SRA737 dose for PK is omitted), the following assessments are completed: complete physical examination, clinical disease assessment, SAE and concomitant medication, WHO performance status and local laboratory assessment of blood (for hematology, biochemistry, and pregnancy testing). At the single-dose PK run-in on Day −7 to Day −4 visit, concomitant medication, vital signs (including temperature, blood pressure, and pulse), height, weight, body surface area (BSA), and WHO performance status are collected. Blood samples are obtained predose for hematology, biochemistry, pregnancy testing, troponin I or T, as well as for tumor markers and tumor profiling. Adverse events (AEs) are collected starting at the administration of SRA737. Archival tissue is submitted for tumor profiling. PK samples are collected at up to 10 time points over a 48-hour time period on Day −7 to −4 (first dose of SRA737 for PK). The sponsor in some cases reduces the requirement for PK sampling, including modification or elimination of the Day −7 to Day −4 visit once sufficient data to evaluate the single-dose PK of SRA737 have been collected and analyzed. Dosing begins on Day 1 with the following procedures occurring at regular intervals:
Adverse event and concomitant medication: On an ongoing basis
Symptom-directed physical exam (if medically indicated): Day 1 of each cycle
Radiological disease assessment: Every 6 weeks after Day 1 for Stage 1 and every 8 weeks after Day 1 for Stage 2
Clinical disease assessment: Every 6 weeks after Day 1 for Stage 1 and every 4 weeks after Day 1 for Stage 2
Tumor markers (serum or urine) (if applicable): every 6 weeks from Cycle 1 Day 1 for Stage 1 and every 4 weeks from Cycle 1 Day 1 for Stage 2
WHO Performance Status and weight/BSA: Day 1 of each cycle
Vital signs: Days 1 and 8 for Stage 1 and Days 1, 8, and 15 for Stage 2
Local laboratory assessment of blood (for hematology, biochemistry, troponin I or T) and urine (urinalysis)—see Section 7.2.2 for detailed schedule
ECHO: Cycle 2 Day 1
ECG: Day 1 of Cycles 1 and 2, and then predose Day 1 at each third subsequent cycle, and Day 1 of any cycle with intra-subject dose escalation
Pharmacokinetics Samples for PK are taken at the following timepoints in Cycle 1: pre-dose on Day 1, pre-dose on Day 10, up to 6 time points post-dose on Day 10. For Stage 2 only, additional samples are taken pre-dose on Day 8 and pre-dose on Day 15. Compliance review of subject diary card.

### Example 6: Phase 1/2 Clinical Study to Confirm Efficacy of SRA737 Monotherapy in Select Tumors with Genetic Alterations that Confer Chk1 Sensitivity

An in-human clinical trial is conducted to confirm efficacy of SRA737 monotherapy methods of treatment and patient selection strategies disclosed herein for prospectively-selected genetically-defined subjects with tumor types known to have a high prevalence of genomic alterations expected to sensitize the tumor to Chk1 inhibition (“Cohort Expansion Phase”). the Cohort Expansion Phase consists of 6 indication-specific expansion cohorts of approximately 20 prospectively-selected genetically-defined subjects each. The cohorts are subjects with previously treated metastatic colorectal cancer [CRC], high grade serous ovarian cancer [HGSOC] without CCNE1 gene amplification, HGSOC with CCNE1 gene amplification (or alternative genetic alteration with similar functional effect), metastatic castration-resistant prostate cancer [mCRPC], advanced non-small cell lung cancer [NSCLC], and squamous cell carcinoma of the head and neck [HNSCC], or squamous cell carcinoma of the anus [SCCA]. Subjects are initially administered SRA737 following the dosing regimen established in Example 4. The dosing regimen in some cases changes during the course of the trial.

Subjects have tumor tissue or ctDNA evidence that their tumor harbors a combination of mutations which are expected to confer sensitivity to Chk1 inhibition. Subjects are selected based on prospective, tumor tissue genetic profiling using NGS.

Expansion cohort subjects have tumors that harbor genomic alterations expected to confer sensitivity to Chk1 inhibition in a minimum of two of the following categories (a)-(e):
a. Key tumor suppressor genes regulating G1 cell cycle progression/arrest such as RB1, TP53, etc. For patients with NHSCC or SCCA, positive HPV status is also considered for eligibility.
b. The DDR pathway including ATM, CDK12, RAD51C. BRCA1, and BRCA2. For patients with CRC, mismatch repair (MMR) genetic alterations and/or high microsatellite instability are also considered for eligibility.
c. Genetic indicators of replicative stress such as gain of function/amplification of Chk1 or ATR or other related gene.
d. Oncogenic drivers such as MYC, KRAS, etc.
e. CCNE1 gene amplification (or alternative genetic alteration with similar functional effect) is required for the CCNE1 gene amplification-specific HGSOC cohort.

In some aspects, subjects meet one of the following criteria (a-e):
a. Metastatic CRC
   i. Histologically and/or cytologically confirmed CRC
   ii. In general, have received at least 1 prior regimen for advanced/metastatic disease
b. HGSOC
   i. Histologically confirmed high grade serous ovarian, fallopian tube or primary peritoneal cancer
   ii. Recurrent platinum-intolerant subjects, or those with platinum-resistant disease, defined as radiological evidence of disease progression within 6 months of the last receipt of platinum-based chemotherapy. Patients with platinum refractory disease (as defined by the European Society for Medical oncology [ESMO] Guidelines) are not eligible.
c. Advanced NSCLC
   i. Locally advanced and recurrent or metastatic, histologically confirmed NSCLC
   ii. In general, have received at least 1 prior regimen for advanced/metastatic disease
d. mCPRC
   i. Histologically or cytologically confirmed adenocarcinoma of the prostate that has progressed after androgen deprivation therapy
e. HNSCC or SCCA
   i. Histologically confirmed HNSCC from any primary site, or SCCA
   ii. For HNSCC: locally advanced disease (i.e., persistent or progressive disease following curative-intent radiation, and not a candidate for surgical salvage due to incurability or morbidity), or metastatic disease
   iii. For SCCA: locally advanced disease or metastatic disease for which no curative intent therapy is available.
   iv. Subjects have received at least 1 prior regimen for advanced/metastatic disease.

Subjects, in general, have measurable disease (per Response Evaluation Criteria in Solid Tumors, version 1.1 [RECIST v1.1]) or, for mCRPC, evaluable disease per any of the following: Measurable disease per RECIST v1.1; increasing prostate specific antigen (PS); or circulating tumor cell (CTC) count of 5 or more cells per 7.5 ml of blood.

Enrollment to Expansion Cohorts in some cases occurs in parallel with the Dose Escalation Phase (see Example 5). A subject that qualifies for the Cohort Expansion Phase is enrolled into an Escalation Cohort whenever possible. Any such subject is considered to have enrolled in both phases simultaneously.

Disease is measured according to the RECIST v1.1 criteria for subjects with solid tumors, according to the revised IWG criteria (Cheson 2007) for subjects with NHL, and for subjects with mCRPC, using a composite of any one of the following: A) Measurable disease per RECIST v1.1; B) Increasing PSA; or C) CTC count of 5 or more cells per 7.5 ml of blood.

Baseline evaluations include radiological measurements of lesions appropriate to the nature of the malignancy. In some cases, this includes: CT scan, liver CT scan, abdominal CT scan, MRI, X-ray, bone scan and/or other radiological measurements as clinically indicated or clinical measurements as appropriate (e.g., assessment of palpable lesions or measurement of tumor markers). All areas of disease present are documented (even if specific lesions are not going to be followed for response) and the dimensions of all measurable lesions are recorded clearly on the scan reports. Any non-measurable lesions are stated as being present. For clinical measurements, documentation by color photography including a ruler to estimate the size of the lesion is strongly recommended, as this aids external independent review of responses.

Tumor assessments is repeated every 8 weeks or more frequently, when clinically indicated. Subjects with bone metastases being followed by bone scans are scanned every 8 weeks (±1 week) for the first 6 months and then every 16 weeks (±2 weeks) thereafter. During Long-term Follow-up, assessments for subjects who have not yet progressed and who have not initiated alternative anti-cancer therapy are done every 16 weeks, unless requested more frequently by the sponsor or investigator. All lesions measured at baseline are measured at every subsequent disease assessment, and recorded clearly on the scan reports. All non-measurable lesions noted at baseline are noted on the scan report as present or absent. All subjects, who are removed from the study treatment for reasons other than progressive disease (PD), should be re-evaluated at the time of treatment discontinuation, unless a tumor assessment was performed within the previous four weeks. Subjects are followed for PD until disease progression or withdrawal from trial.

All subjects who have measurable disease, receive at least one cycle of SRA737 and have a baseline plus at least 1 post-baseline assessment of disease are evaluable for response. Subjects who develop clear evidence of PD without a formal disease assessment and those without a formal disease assessment before study withdrawal are considered non-responders. Complete responses and PRs are required to be confirmed by a subsequent assessment at least 4 weeks later. Stable Disease (SD) determination requires that the relevant criteria be met at least once, a minimum of 6 weeks after the initial dose of SRA737 is given.

Should rapid tumor progression occur before the completion of 4 weeks of treatment, the subject is classified as having early progression.

Tumor response should be classified as “not evaluable” (NE), only when it is not possible to classify it under another response category, for example, when baseline and/or follow-up assessment is not performed or not performed appropriately.

Response criteria are defined below:
a. Complete Response (CR): Disappearance of all target lesions. Any pathological lymph nodes (whether target or non-target), in general, have reduction in short axis to <10 mm.
b. Partial Response (PR): At least a 30% decrease in the sum of diameters of target lesions, taking as reference the baseline sum diameters.
c. Progressive Disease (PD): At least a 20% increase in the sum of diameters of target lesions, taking as reference the smallest sum on study (this includes the baseline sum if that is the smallest on study). In addition to the relative increase of 20%, the sum, in general, also demonstrates an absolute increase of at least 5 mm. (Note: the appearance of one or more new lesions is also considered progression).
d. Stable Disease (SD): Neither sufficient shrinkage to qualify for PR nor sufficient increase to qualify for PD, taking as reference the smallest sum diameters while on study.

The results of this study demonstrate the efficacy of SRA737 monotherapy for the treatment of tumors with genetic alterations that confer sensitivity to Chk1 inhibition.

### Example 7: Phase 1/2 Clinical Study to Confirm Efficacy of SRA737 Combination Therapy in Select Tumors with Genetic Alterations that Confer Chk1 Sensitivity

An in-human clinical trial is conducted to confirm efficacy of SRA737 combination therapy methods of treatment and patient selection strategies disclosed herein for prospectively-selected genetically-defined subjects with tumor types known to have a high prevalence of genomic alterations expected to sensitize the tumor to Chk1 inhibition (“Cohort Expansion Phase”). In the Cohort Expansion Phase, approximately 20 prospectively-selected genetically-defined subjects are enrolled in each of 4 indication-specific cohorts: high-grade serous ovarian cancer (HGSOC), small cell lung cancer (SCLC), soft tissue sarcoma (STS), and cervical/anogenital cancer. Based on the PK data that established dosing resulting in an efficacious concentration of SRA737 (see Example 5), a starting dose level of 500 mg SRA737 and 100 mg/m² gemcitabine was used. The dosing regimen in some cases changes during the course of the trial. SRA737 capsules are taken on an empty stomach (subjects fast for at least 2 hours pre- and 1 hour post-administration), unless otherwise instructed.

Subjects have:
a. Histologically or cytologically proven advanced malignancy of the following types, for which no other conventional therapy is considered appropriate:
   i. High-grade serous ovarian cancer (HGSOC)
      1. Histologically confirmed high-grade serous ovarian, fallopian tube, or primary peritoneal cancer
      2. Platinum-resistant or refractory disease, or if the subject is intolerant to platinum therapy
   ii. Small cell lung cancer
      1. In general, have received at least 1 but no more than 3 prior regimens for advanced disease, unless otherwise approved by sponsor
   iii. Soft tissue sarcoma
      1. Including undifferentiated pleiomorphic sarcoma/malignant fibrous histiocytoma (MFH) (including high-grade spindle cell sarcoma/pleomorphic liposarcomas), leiomyosarcoma, and dedifferentiated liposarcomas. Other types of STS in some cases are eligible with sponsor's approval
      2. In general, have received at least 1 but no more than 3 prior regimens for advanced disease, unless otherwise approved by sponsor
   iv. Cervical/anogenital cancer
      1. Including all cervical carcinoma and advanced/metastatic squamous cell carcinoma of the anus, penis, vagina, and vulva
      2. In general, have received at least 1 but no more than 3 prior regimens for advanced disease, unless otherwise approved by sponsor
   v. Urothelial Carcinoma
      1. Histologically confirmed locally advanced and unresectable or metastatic urothelial carcinoma of the bladder, upper urinary tract or urethra
      2. In general, have received at least 1 but no more than 3 prior regimens for advanced disease.
b. Measurable disease per RECIST v1.1 (see below)
c. Subjects have predicted sensitivity to Chk1 inhibition based on factors including: genetic profiling of tumor tissue or ctDNA, HPV status, and germline BRCA1 and BRCA2 gene status. All subjects have genetic profiling from tumor tissue or ctDNA; profiling is performed prospectively if required to evaluate Chk1 sensitivity or otherwise performed retrospectively
   i. For subjects with HGSOC, documented somatic or germline BRCA1 and BRCA2 wild-type status confer eligibility without requirement for prospective genetic profiling. If documented BRCA status is not available, genetic profiling in some cases are performed prospectively to determine eligibility.
   ii. Subjects with SCLC are eligible without requirement for prospective genetic profiling on the basis of very high prevalence of cancer related alterations in the tumor suppressor genes (eg, TP53 and RB1) in this population.
   iii. For subjects with STS, and any others for whom genetic profiling is performed prospectively, eligibility is determined by the sponsor's review of genetic abnormalities detected in genes in the following categories:
      Key tumor suppressor genes regulating GI cell cycle progression/arrest such as RB1, TP53, etc. For relevant cancers, positive human papilloma virus (HPV) status is also considered for eligibility.
         a. The DNA damage response pathway including ATM, CDK12, RAD51C. BRCA1, BRCA2, mismatch repair genetic alterations and/or high microsatellite instability.
         b. Genetic indicators of replicative stress such as gain of function/amplification of Chk1 or ATR or other related gene.
         c. Oncogenic drivers such as MYC, CCNE1, etc.
   iv. For subjects with anogenital cancer, known HPV positive status in some cases confers eligibility without requirement for prospective genetic profiling. If HPV status is not known or not positive, genetic profiling (or HPV testing where appropriate) in some cases is performed prospectively to determine eligibility. Subjects with cervical cancer or squamous cell carcinoma of the anus are eligible without requirement for prospective genetic profiling based on the very high prevalence of HPV positivity in these populations.

Alternatively, subjects have one of the histologically or cytologically proven advanced malignancies described above and tumor tissue or ctDNA evidence that their tumor harbors one or more mutations that are expected to confer sensitivity to Chk1 inhibition. Eligibility will be determined by the sponsor's review of genetic abnormalities detected in genes in the following categories:
a. Key tumor suppressor genes regulating G1 cell cycle progression/arrest such as RB1, TP53, etc. For relevant cancers, positive human papilloma virus (HPV) status is also considered for eligibility.
b. The DNA damage response pathway including ATM, CDK12, RAD51C. BRCA1, BRCA2, mismatch repair genetic alterations and/or high microsatellite instability.
c. Genetic indicators of replicative stress such as gain of function/amplification of Chk1 or ATR or other related gene.
d. Oncogenic drivers such as MYC, KRAS, etc.

Subjects are excluded based on the following criteria:
a. Received the following prior or current anticancer therapy in the timeframes noted prior to receiving SRA737 and have recovered from toxicity:
   i. Radiotherapy, chemotherapy, PARP inhibitors, other targeted therapies, or other IMPs within 2 weeks
   ii. Nitrosoureas or Mitomycin C within 6 weeks
   iii. Any prior treatment with a Chk1 inhibitor at any point or prior treatment with an ATR inhibitor within 6 months
b. No more than 3 previous treatment regimens for advanced disease (not applicable to HGSOC expansion cohort)
c. Other malignancy within the past 2 years, except for adequately treated tumors
d. If, in the opinion of the Investigator, the subject is highly likely to experience clinically significant myelosuppression
e. Ongoing toxic manifestations of previous treatments greater than NCI-CTCAE Grade 1
f. History of allergy to gemcitabine
g. New or progressing brain metastases. Subjects with brain metastases that have been asymptomatic and radiologically stable over an 8-week period and have not been treated with steroids during that time in some cases are included with approval from the sponsor.
h. High medical risk because of nonmalignant systemic disease
i. Serologically positive for hepatitis B, hepatitis C or HIV
j. Serious cardiac condition, left ventricular ejection fraction <45% at baseline, history of cardiac ischemia within the past 6 months, or prior history of cardiac arrhythmia requiring treatment, unless approved by the sponsor.
k. Prior bone marrow transplant or extensive radiotherapy to greater than 25% of bone marrow within the previous 8 weeks
l. Peanut allergy
m. QTcF >450 msec in adult makes and >470 msec in adult females
n. Impairment of gastrointestinal (GI) function or GI disease that in some cases significantly alter the absorption of SRA737
o. Inability to swallow capsules without chewing or crushing
p. Is a participant or plans to participate in another interventional clinical trial
q. Any other condition which in the Investigator's opinion would not make the subject a good candidate

All enrolled subjects who have measurable disease, receive at least 75% (Stage 1) or 83% (Stage 2) of SRA737 in 1 cycle (or the equivalent if the sponsor elects to evaluate an alternative dosing schedule), and have a baseline assessment of disease plus at least 1 postbaseline disease assessment are evaluated for response. All subjects who enroll into the Cohort Expansion Phase are evaluated for response if they have measurable disease, receive at least 1 cycle of study medication as defined above, have a baseline assessment of disease plus at least 1 postbaseline disease assessment and are confirmed as having met the genetic selection requirements.

In addition, subjects who have measurable disease and received at least 83% of SRA737 (if the sponsor elects to evaluate an alternative dosing schedule) in 1 cycle but developed PD, intolerable toxicity, or death prior to the postbaseline assessment are also evaluable and are classified as non-responders.

The analysis of all efficacy endpoints is based on the Response Evaluable Population and will be evaluated using RECIST v1.1 criteria, as described below.

Other endpoints include: Duration of response (DOR), Disease control rate (DCR), Time to response (TTR), PFS, Time to Progression (TTP), OS. Other exploratory objectives are described in Table 9.

TABLE 9
EXPLORATORY OBJECTIVES AND ENDPOINTS

| Exploratory Objectives | Endpoints |
|---|---|
| To assess the relationship between response and | Objective response rate as measured by RECIST |
| the presence of selected genetic alternations | v1.1 and gene alterations in tumor tissue or ctDNA |
| detected in tumor tissue or circulating tumor | at baseline as measured by next generation |
| deoxyribonucleic acid (ctDNA). | sequencing (NGS). |
| To explore possible clinical predictors of | Characteristics such as performance status, prior |
| outcomes. | therapy, indication and other known or potential |
| | prognostic or predictive factors. |
| To investigate the PDn of SRA737 in combination | Proof of target engagement and changes in |
| with gemcitabine in tumor tissue. | mechanism of action biomarkers between baseline |
| | and on treatment with SRA737, including, but not |
| | limited to: pSer296 Chk1, pS317 Chk1, and total |
| | Chk1. |
| To investigate the PDn of SRA737 in combination | Proof of target engagement and changes in |
| with gemcitabine in surrogate tissues such as blood | mechanism of action biomarkers between baseline |
| or peripheral blood mononuclear cell (PBMCs). | and on treatment with SRA737, including but not |
| | limited to: Comet assay, pS296 Chk1, pS317 |
| | Chk1, pS345 Chk1, total Chk1, gammaH2AX and |
| | RAD51. |

Additional trials are conducted with SRA737 in combination with other therapies, including administering a chemotherapeutic agent, administering an antibody or antibody fragment, administering a radiation treatment, administering an external inducer of replication stress, or administering a combination thereof. Other trials are conducted with SRA737 in combination with other therapies, including administering olaparib, niraparib, rucaparib, talazoparib, cisplatin, a ribonucleotide reductase inhibitor, etoposide, SN-38/CPT-11, mitomycin C, or combinations thereof.

The results of this study demonstrate the efficacy of SRA737 combination therapy for the treatment of tumors with genetic alterations that confer sensitivity to Chk1 inhibition.

### RECIST Criteria

Assessment of disease response in this study are performed according to the revised RECIST criteria v1.1. RECIST criteria are described in greater detail in Eisenhauer, et al. (New response evaluation criteria in solid tumours: Revised RECIST guideline (version 1.1). Eur J Cancer [Internet]. 2009), herein incorporated by reference for all it teaches.

At baseline, tumor lesions/lymph nodes are generally categorized measurable or non-measurable as follows:

Measurable

Tumor lesions: are generally accurately measured in at least one dimension (longest diameter in the plane of measurement is to be recorded) with a minimum size of:
10 mm by CT scan (CT scan slice thickness no greater than 5 mm; see Appendix II in Eisenhauer, et al., [Eisenhauer, 2009] on imaging guidance)
10 mm caliper measurement by clinical exam (lesions which cannot be accurately measured with calipers should be recorded as non-measurable)
20 mm by chest X-ray

Malignant lymph nodes: To be considered pathologically enlarged and measurable, a lymph node is generally 15 mm in the short axis when assessed by CT scan (CT scan slice thickness recommended to be no greater than 5 mm). At baseline and in follow-up, only the short axis is generally measured and followed.

Non-Measurable

All other lesions, including small lesions (longest diameter <10 mm or pathological lymph nodes with >10 to <15 mm short axis) as well as truly non-measurable lesions. Lesions considered truly non-measurable generally include: leptomeningeal disease, ascites, pleural or pericardial effusion, inflammatory breast disease, lymphangitic involvement of skin or lung, abdominal masses/abdominal organomegaly identified by physical exam that is not measurable by reproducible imaging techniques.

Bone lesions, cystic lesions, and lesions previously treated with local therapy require particular comment:

Bone lesions:
Bone scan, PET scan or plain films are generally not considered adequate imaging techniques to measure bone lesions. However, these techniques can be used to confirm the presence or disappearance of bone lesions.
Lytic bone lesions or mixed lytic-blastic lesions, with identifiable soft tissue components, that can be evaluated by cross sectional imaging techniques such as CT or MRI are generally considered as measurable lesions if the soft tissue component meets the definition of measurability described above.
Blastic bone lesions are generally non-measurable.

Cystic lesions:
Lesions that meet the criteria for radiographically defined simple cysts are generally not considered as malignant lesions (neither measurable nor non-measurable) since they are, by definition, simple cysts.
‘Cystic lesions’ thought to represent cystic metastases are generally considered as measurable lesions, if they meet the definition of measurability described above. However, if non-cystic lesions are present in the same subject, these are preferred for selection as target lesions.

Lesions with prior local treatment:
Tumor lesions situated in a previously irradiated area, or in an area subjected to other loco-regional therapy, are usually not considered measurable unless there is demonstrated progression in the lesion. Study protocols generally detail the conditions under which such lesions are generally considered measurable.

Method of Assessment

All measurements are generally recorded in metric notation, using calipers if clinically assessed. All baseline evaluations are generally performed as close as possible to the treatment start and never more than 4 weeks before the beginning of the treatment.

The same method of assessment and the same technique are generally used to characterize each identified and reported lesion at baseline and during follow-up. Imaging based evaluation are generally always done rather than clinical examination unless the lesion(s) being followed cannot be imaged but are assessable by clinical exam.

Clinical lesions are generally considered measurable when they are superficial and ≥10 mm diameter as assessed using calipers (e.g. skin nodules). For the case of skin lesions, documentation by color photography including a ruler to estimate the size of the lesion is suggested. As noted above, when lesions can be evaluated by both clinical exam and imaging, imaging evaluation is generally undertaken since it is more objective and in some cases is also reviewed at the end of the study.

Chest CT is generally preferred over chest X-ray, particularly when progression is an important endpoint, since CT is more sensitive than X-ray, particularly in identifying new lesions. However, in some cases, lesions on chest X-ray are considered measurable if they are clearly defined and surrounded by aerated lung

CT is generally the best currently available and reproducible method to measure lesions selected for response assessment. This guideline has defined measurability of lesions on CT scan based on the assumption that CT slice thickness is 5 mm or less. When CT scans have slice thickness greater than 5 mm, the minimum size for a measurable lesion is twice the slice thickness. MRI is also acceptable in certain situations (e.g. for body scans). More details concerning the use of both CT and MRI for assessment of objective tumor response evaluation are provided in the publication from Eisenhauer et al.

Ultrasound is generally not useful in assessment of lesion size and is generally not used as a method of measurement. Ultrasound examinations, in general, cannot be reproduced in their entirety for independent review at a later date and, because they are operator dependent, it generally cannot be guaranteed that the same technique and measurements will be taken from one assessment to the next (described in greater detail in Eisenhauer, et al. (2009). If new lesions are identified by ultrasound in the course of the study, confirmation by CT or MRI is generally advised. If there is concern about radiation exposure at CT, MRI in some cases is used instead of CT in selected instances.

The utilization of endoscopy and laparoscopy techniques for objective tumor evaluation is generally not advised. However, they are, in general, useful to confirm complete pathological response when biopsies are obtained or to determine relapse in trials where recurrence following complete response or surgical resection is an endpoint.

Tumor markers alone are generally not used to assess objective tumor response. If markers are initially above the upper normal limit, however, they are generally normalized for a subject to be considered in complete response.

Cytology and histology are generally used to differentiate between PR and CR in rare cases if required by protocol (for example, residual lesions in tumor types such as germ cell tumors, where known residual benign tumors can remain). When effusions are known to be a potential adverse effect of treatment (e.g. with certain taxane compounds or angiogenesis inhibitors), the cytological confirmation of the neoplastic origin of any effusion that appears or worsens during treatment are generally considered if the measurable tumor has met criteria for response or stable disease in order to differentiate between response (or stable disease) and progressive disease.

Tumor Response Evaluation

To assess objective response or future progression, the overall tumor burden at baseline is generally estimated and used as a comparator for subsequent measurements. Measurable disease is generally defined by the presence of at least one measurable lesion.

When more than one measurable lesion is present at baseline all lesions up to a maximum of five lesions total (and a maximum of two lesions per organ) representative of all involved organs are generally identified as target lesions and are recorded and measured at baseline (this means in instances where subjects have only one or two organ sites involved a maximum of two and four lesions respectively are recorded). Target lesions are generally selected on the basis of their size (lesions with the longest diameter) and are generally representative of all involved organs, but in addition are generally those that lend themselves to reproducible repeated measurements. In some cases, the largest lesion does not lend itself to reproducible measurement in which circumstance the next largest lesion which can be measured reproducibly is generally selected, as exemplified in FIG. 3 Eisenhauer, et al. (2009).

Lymph nodes merit special mention since they are normal anatomical structures which in some cases are visible by imaging even if not involved by tumor. Pathological nodes which are defined as measurable and in some cases are identified as target lesions, in general, meets the criterion of a short axis of ≥15 mm by CT scan. Only the short axis of these nodes generally contributes to the baseline sum. The short axis of the node is generally the diameter normally used by radiologists to judge if a node is involved by solid tumor. Nodal size is normally reported as two dimensions in the plane in which the image is obtained (for CT scan this is almost always the axial plane; for MRI the plane of acquisition in some cases are axial, sagital or coronal). The smaller of these measures is the short axis. For example, an abdominal node which is reported as being 20 mm×30 mm has a short axis of 20 mm and qualifies as a malignant, measurable node. In this example, 20 mm should be recorded as the node measurement. All other pathological nodes (those with short axis 210 mm but <15 mm) are generally considered non-target lesions. Nodes that have a short axis <10 mm are generally considered non-pathological and are generally not recorded or followed.

A sum of the diameters (longest for non-nodal lesions, short axis for nodal lesions) for all target lesions is generally calculated and reported as the baseline sum diameters. If lymph nodes are to be included in the sum, then as noted above, only the short axis is added into the sum. The baseline sum diameters are generally used as reference to further characterize any objective tumor regression in the measurable dimension of the disease.

All other lesions (or sites of disease) including pathological lymph nodes are generally identified as non-target lesions and are generally recorded at baseline. Measurements are generally not required and these lesions are generally followed as ‘present’, ‘absent’, or in rare cases ‘unequivocal progression’ (more details to follow). In addition, it is possible to record multiple non-target lesions involving the same organ as a single item on the case record form (e.g. ‘multiple enlarged pelvic lymph nodes’ or ‘multiple liver metastases’).

Response Criteria

Complete Response (CR): Disappearance of all target lesions. Any pathological lymph nodes (whether target or non-target) are reduced in short axis to <10 mm.

Partial Response (PR): At least a 30% decrease in the sum of diameters of target lesions, taking as reference the baseline sum diameters.

Progressive Disease (PD): At least a 20% increase in the sum of diameters of target lesions, taking as reference the smallest sum (this includes the baseline sum if that is the smallest on study). In addition to the relative increase of 20%, the sum generally demonstrates an absolute increase of at least 5 mm. (Note: the appearance of one or more new lesions is generally also considered progression).

Stable Disease (SD): Neither sufficient shrinkage to qualify for PR nor sufficient increase to qualify for PD, taking as reference the smallest sum diameters.

Lymph nodes identified as target lesions generally record the actual short axis measurement (measured in the same anatomical plane as the baseline examination), generally even if the nodes regress to below 10 mm. This means that when lymph nodes are included as target lesions, the ‘sum’ of lesions in some cases are not be zero even if complete response criteria are met, since a normal lymph node is generally defined as having a short axis of <10 mm. Case report forms or other data collection methods in some cases are therefore designed to have target nodal lesions recorded in a separate section where, in order to qualify for CR, each node generally achieves a short axis <10 mm. For PR, SD and PD, the actual short axis measurement of the nodes is preferably included in the sum of target lesions.

While on study, all lesions (nodal and non-nodal) recorded at baseline generally record their actual measurements at each subsequent evaluation, even when very small (e.g. 2 mm). However, sometimes lesions or lymph nodes which are recorded as target lesions at baseline become so faint on CT scan that the radiologist in some cases does not feel comfortable assigning an exact measure and in some cases report them as being ‘too small to measure’. When this occurs it is, in general, important that a value is recorded on the case report form. If it is the opinion of the radiologist that the lesion has likely disappeared, the measurement is generally recorded as 0 mm. If the lesion is believed to be present and is faintly seen but too small to measure, a default value of 5 mm is generally assigned. (Note: It is, in general, less likely that this rule is used for lymph nodes since they usually have a definable size when normal and are frequently surrounded by fat such as in the retroperitoneum; however, if a lymph node is believed to be present and is faintly seen but too small to measure, a default value of 5 mm is generally assigned in this circumstance as well). This default value is derived from the 5 mm CT slice thickness (but generally is not changed with varying CT slice thickness). The measurement of these lesions is potentially non-reproducible, therefore providing this default value generally prevents false responses or progressions based upon measurement error. To reiterate, however, if the radiologist is able to provide an actual measure, that is generally recorded, even if it is below 5 mm.

When non-nodal lesions ‘fragment’, the longest diameters of the fragmented portions are generally added together to calculate the target lesion sum. Similarly, as lesions coalesce, a plane between them are generally maintained that would aid in obtaining maximal diameter measurements of each individual lesion. If the lesions have truly coalesced such that they are no longer separable, the vector of the longest diameter in this instance generally is the maximal longest diameter for the ‘coalesced lesion’.

While some non-target lesions in some cases are actually measurable, they generally are not measured and instead are generally assessed only qualitatively at the time points specified in the protocol.

Complete Response (CR): Disappearance of all non-target lesions and normalization of tumor marker level. All lymph nodes are non-pathological in size (<10 mm short axis).

Non-CR/Non-PD: Persistence of one or more non-target lesion(s) and/or maintenance of tumor marker level above the normal limits.

Progressive Disease (PD): Unequivocal progression (see comments below) of existing non-target lesions. (Note: the appearance of one or more new lesions is also considered progression).

When the subject also has measurable disease, to achieve ‘unequivocal progression’ on the basis of the non-target disease, there generally is an overall level of substantial worsening in non-target disease such that, even in presence of SD or PR in target disease, the overall tumor burden has increased sufficiently to merit discontinuation of therapy. A modest ‘increase’ in the size of one or more non-target lesions is usually not sufficient to quality for unequivocal progression status. The designation of overall progression solely on the basis of change in non-target disease in the face of SD or PR of target disease is generally therefore extremely rare.

A subject having only non-measurable disease arises in some Phase III trials when it is not a criterion of study entry to have measurable disease. The same general concepts apply here as noted above, however, in this instance there is no measurable disease assessment to factor into the interpretation of an increase in non-measurable disease burden. Because worsening in non-target disease is generally not easily quantified (by definition: if all lesions are truly non-measurable) a useful test that can generally be applied when assessing subjects for unequivocal progression is to consider if the increase in overall disease burden based on the change in non-measurable disease is comparable in magnitude to the increase that would be required to declare PD for measurable disease: i.e. an increase in tumor burden representing an additional 73% increase in ‘volume’ (which is equivalent to a 20% increase diameter in a measurable lesion). Examples include an increase in a pleural effusion from ‘trace’ to ‘large’, an increase in lymphangitic disease from localized to widespread, or in some cases are described in protocols as ‘sufficient to require a change in therapy’. If ‘unequivocal progression’ is seen, the subject is generally considered to have had overall PD at that point. While it would be ideal to have objective criteria to apply to non-measurable disease, the very nature of that disease makes it generally very difficult to do so; therefore the increase generally is substantial.

The appearance of new malignant lesions generally denotes disease progression; therefore, some comments on detection of new lesions are generally important. There are generally no specific criteria for the identification of new radiographic lesions; however, the finding of a new lesion generally is unequivocal: i.e., generally not attributable to differences in scanning technique, change in imaging modality or findings thought to represent something other than tumor (for example, some ‘new’ bone lesions in some cases are simply healing or flare of pre-existing lesions). This is particularly important when the subject's baseline lesions show partial or complete response. For example, necrosis of a liver lesion is frequently reported on a CT scan report as a ‘new’ cystic lesion, which it generally is not.

A lesion identified on a follow-up study in an anatomical location that was not scanned at baseline is generally considered a new lesion and generally indicates disease progression. An example of this is the subject who has visceral disease at baseline and while on study has a CT or MRI brain ordered which reveals metastases. The subject's brain metastases are generally considered to be evidence of PD even if he/she did not have brain imaging at baseline.

If a new lesion is equivocal, for example because of its small size, continued therapy and follow-up evaluation generally clarifies if it represents truly new disease. If repeat scans confirm there is definitely a new lesion, then progression is generally declared using the date of the initial scan.

While FDG-PET response assessments need additional study, it is sometimes reasonable to incorporate the use of FDG-PET scanning to complement CT scanning in assessment of progression (particularly possible ‘new’ disease). New lesions on the basis of FDG-PET imaging are generally identified according to the following algorithm:

a. Negative FDG-PET at baseline, with a positive* FDG-PET at follow-up is generally a sign of PD based on a new lesion. (* A ‘positive’ FDG-PET scan lesion generally means one which is FDG avid with an uptake greater than twice that of the surrounding tissue on the attenuation corrected image.)

b. No FDG-PET at baseline and a positive FDG-PET at follow-up:
If the positive FDG-PET at follow-up corresponds to a new site of disease confirmed by CT, this is generally PD.
If the positive FDG-PET at follow-up is not confirmed as a new site of disease on CT, additional follow-up CT scans are generally performed to determine if there is truly progression occurring at that site (if so, the date of PD will be the date of the initial abnormal FDG-PET scan). A ‘positive’ FDG-PET scan lesion generally means one which is FDG avid with an uptake greater than twice that of the surrounding tissue on the attenuation corrected image.
If the positive FDG-PET at follow-up corresponds to a pre-existing site of disease on CT that is not progressing on the basis of the anatomic images, this is generally not PD.

Evaluation of Best Overall Response

The best overall response is generally the best response recorded from the start of the study treatment until the end of treatment. Should a response not be documented until after the end of therapy in this trial, post-treatment assessments generally are considered in the determination of best overall response as long as no alternative anti-cancer therapy has been given. The subject's best overall response assignment generally depends on the findings of both target and non-target disease and generally also takes into consideration the appearance of new lesions.

It is generally assumed that at each protocol-specified time point, a response assessment occurs. Table 10 provides a summary of the overall response status calculation at each time point for subjects who have measurable disease at baseline.

When subjects have non-measurable (therefore non-target) disease only, Table 11 is generally used.

When no imaging/measurement is done at all at a particular time point, the subject is generally not evaluable (NE) at that time point. If only a subset of lesion measurements are made at an assessment, usually the case is generally also considered NE at that time point, unless a convincing argument is made that the contribution of the individual missing lesion(s) does not change the assigned time point response. This would be most likely to happen in the case of PD. For example, if a subject had a baseline sum of 50 mm with three measured lesions and at follow-up only two lesions were assessed, but those gave a sum of 80 mm, the subject has generally achieved PD status, regardless of the contribution of the missing lesion.

The best overall response is generally determined once all the data for the subject is known.

Best response determination in trials where confirmation of complete or partial response is generally not required: Best response in these trials is generally defined as the best response across all time points (for example, a subject who has SD at first assessment, PR at second assessment, and PD on last assessment has a best overall response of PR). When SD is believed to be best response, it, in general, also meets the protocol specified minimum time from baseline. If the minimum time is not met when SD is otherwise the best time point response, the subject's best response generally depends on the subsequent assessments. For example, a subject who has SD at first assessment, PD at second and does not meet minimum duration for SD, will have a best response of PD. The same subject lost to follow-up after the first SD assessment is generally considered inevaluable.

When nodal disease is included in the sum of target lesions and the nodes decrease to ‘normal’ size (<10 mm), in some cases they still have a measurement reported on scans. This measurement is generally recorded even though the nodes are normal in order not to overstate progression should it be based on increase in size of the nodes. As noted earlier, this means that subjects with CR in some cases do not have a total sum of ‘zero’ on the case report form (CRF).

Subjects with a global deterioration of health status requiring discontinuation of treatment without objective evidence of disease progression at that time generally are reported as ‘symptomatic deterioration’. Every effort is generally made to document objective progression even after discontinuation of treatment. Symptomatic deterioration is generally not a descriptor of an objective response: it is a reason for stopping study therapy. The objective response status of such subjects is generally determined by evaluation of target and non-target disease as shown in Tables 10 and 11.

Conditions that define ‘EP, early death and inevaluability’ are study specific and are generally clearly described in each protocol (depending on treatment duration, treatment periodicity).

In some circumstances it is difficult to distinguish residual disease from normal tissue. When the evaluation of complete response depends upon this determination, it is generally recommended that the residual lesion be investigated (fine needle aspirate/biopsy) before assigning a status of complete response. In some cases, FDG-PET is used to upgrade a response to a CR in a manner similar to a biopsy in cases where a residual radiographic abnormality is thought to represent fibrosis or scarring.

For equivocal findings of progression (e.g. very small and uncertain new lesions; cystic changes or necrosis in existing lesions), treatment in some cases continues until the next scheduled assessment. If at the next scheduled assessment, progression is confirmed, the date of progression generally is the earlier date when progression was suspected.

TABLE 10
Time point response: subjects with target (target) disease

| Target lesions | Non-target lesions | New lesions | Overall response |
|---|---|---|---|
| CR | CR | No | CR |
| CR | Non-CR/non-PD | No | PR |
| CR | Not evaluated | No | PR |
| PR | Non-PD or | No | PR |
| | not all evaluated | | |
| SD | Non-PD or | No | SD |
| | not all evaluated | | |
| Not all evaluated | Non-PD | No | NE |
| PD | Any | Yes or No | PD |
| Any | PD | Yes or No | PD |
| Any | Any | Yes | PD |

CR = complete response,
PR = partial response,
SD = stable disease,
PD = progressive disease, and
NE not evaluable.

TABLE 11
Time point response: subjects with non-target disease only

| Non-target lesions | New lesions | Overall response |
|---|---|---|
| CR | No | CR |
| Non-CR/non-PD | No | Non-CR/non-PD(a) NE |
| Not all evaluated | No | PD |
| Unequivocal PD | Yes or No | PD |
| Any | Yes | PD |

CR = complete response,
PD = progressive disease, and NE not evaluable.
(a) 'Non-CR/non-PD' is preferred over 'stable disease' for non-target disease since SD is increasingly used as endpoint for assessment of efficacy in some trials so to assign this category when no lesions can be measured is not advised.

Duration of Response

The duration of overall response is generally measured from the time measurement criteria are first met for CR/PR (whichever is first recorded) until the first date that recurrent or progressive disease is recorded on study).

The duration of overall complete response is generally measured from the time measurement criteria are first met for CR until the first date that recurrent disease is objectively documented.

Stable disease is generally measured from the start of the treatment (in randomized trials, from date of randomization) until the criteria for progression are met, taking as reference the smallest sum on study (if the baseline sum is the smallest, this is the reference for calculation of PD).

### Example 8: Materials and Methods

This example presents general materials and methods utilized in examples 9-12.

Cell lines were obtained from ATCC. All cell lines were tested and authenticated by short tandem repeat profiling (DNA Fingerprinting) within 6 months of the study and routinely tested for Mycoplasma species before any experiments were performed.

For cell cultures, cell lines were grown in RPMI, unless otherwise mentioned by the provider, with 10% fetal bovine serum and antibiotics, cultured at 37° C. in a humidified chamber with 5% CO₂. All cell lines included in the study were profiled at passage 4-8 to abrogate the heterogeneity introduced by long-term culture.

Materials

Gemcitabine was obtained from the MD Anderson's Pharmacy. SRA737 was provided by Sierra Oncology.

Cell Viability Assay

SCLC cell lines were incubated with dimethyl sulfoxide (vehicle control), SRA737 or Gemcitabine for 96 hours at nine distinct concentrations, with the maximum dose being 10 μM. A CellTiter-Glo luminescent cell viability assay (Promega) was performed after 96 hours of treatment as per the manufacturer's specifications. IC50 values were estimated using Drexplorer, as previously described (Tong P, Coombes K R, Johnson F M, Byers L A, Diao L, Liu D D, Lee J J, Heymach J V, Wang J. drexplorer: A tool to explore dose-response relationships and drug-drug interactions. Bioinformatics. 2015 May 15; 31(10):1692-4.)

RNA Isolation

RNA was isolated using the Direct-zol RNA MiniPrep Kit (Zymo Research, cat #R2050) according to the manufacturer's instructions. RNA concentrations were determined using a NanoDrop 2000 UV-Vis spectrophotometer (Thermo Scientific).

Quantitative PCR (qPCR)

Reverse transcription reactions were carried out using SuperScript III First-Strand Synthesis SuperMix (Invitrogen, cat #18080-400).

Real-time PCR was done using SYBR Select Master Mix (Life Technologies, cat #4472908) according to the manufacturer's protocol. Primers were purchased from Sigma Aldrich, Inc. Triplicate PCR reactions were run on ABI (7500 Fast Real Time PCR System). The comparative Ct method using the average 2ΔΔCT value for each set of triplicates was used, and the average of the biological replicates was calculated. Negative controls were included for every primer set, and GAPDH was used as the positive control.

Preparation of Protein Lysates

Protein lysate was collected from sub confluent cultures after 24-hr in full-serum media (10% fetal bovine serum or FBS). The lysates were collected as described previously.

Western Blot Analysis

Western blot analysis was performed using SDS-PAGE followed by transfer to nitrocellulose membrane using the BioRad Gel system. Membranes were incubated in the following primary antibodies (1:1000) overnight: PD-L1 (CST), total and phospho- (S366) STING (CST), total and phospho- (S396) IRF3 (CST), phospho-YH2AX (CST) and Actin (Sigma). Secondary anti-rabbit, HRP-linked antibodies were purchased from Cell Signaling Biosciences and detected using the Chemidoc imaging system mage-captured with Image Studio Version 3.1 software.

Reverse-Phase Protein Array (RPPA)

RPPAs were printed from lysates as previously described. The quality of the antibodies was validated by Western Blots and correlation of protein levels in previous RPPA experiments were determined, as previously described. The RPPA samples were analyzed as described before.

Tumor Growth Assessment

Tumors were evaluated twice weekly for the duration of the study. Tumor volumes were measured on all mice three times per week and calculated (width²×length×0.4) using manual calipers. Once the average tumor size was in the range of 120-150 mm³, mice were randomized into dosing groups using stratified sampling by assigning three animals per group for short-term reverse-phase protein array analysis and long-term treatment. Dosing schedules and duration varied depending on the study. Mice were weighed three times per week for the duration of the study, and a decrease in body weight >15% was considered indicative of a toxic dose. The Student t-test was used to determine statistical significance between compound- and vehicle-treated groups.

Flow Cytometry

Single-cell suspensions were prepared and stained according to standard protocols for flow cytometry with antibodies. For intracellular staining, cells were fixed and permeabilized with BD Cytofix/Cytoperm (BD Biosciences). The data were acquired on a Fortessa or Calibur platform (BD Biosciences) and analyzed with FlowJo software (version 7.6; Tree Star). For analyzing the abundance and the function of CD4+ or CD8+ TILs, single-cell suspensions were prepared from tumors and spleen and stained; the staining of spleen cells was used as the reference of lymphocyte gating, then CD3+ cells were gated, and then CD4+ or CD8+ population was analyzed.

Histologic Analysis

For immunohistochemistry, cryosections (8 μm) of tumor tissues were fixed with acetone and stained with antibody against mouse CD4 (Sinobiological, Cat. no. 50134-R001) and CD8 (eBioscience, Cat. No. 14-0808-82) and horseradish peroxidase-conjugated secondary antibody. The image of the entire stained tumor section was scanned with an Olympus BX41 microscope and a semi-quantitative immunohistochemical score was determined by visual examination of the entire stained section of the tumor. The score between 0-6 was assigned by taking into consideration the intensity of positive staining within the tumor section and the prevalence of positive staining within the entire tumor section.

Micronuclei Assay

H69 cells were cultured as discussed earlier and treated with or without SRA737 (1 μM). Cytochalasin B was added at 20^{th} hour to each culture to give a final concentration of 3 μg/ml and the culture was incubated at 37° C. for up to 24 hrs. After 24 hrs incubation, the cells were centrifuged at 1000 rpm for 5 min. The supernatant was removed and the pellet was treated with weak hypotonic solution (0.075 M KCl/0.9% Saline, 1:9) and incubated at 37° C. for 5 min. After this, the cells were centrifuged and the pellets were fixed in fresh fixative (methanol:acetic acid, 3:1). Cells were dropped onto glass slides were prepared and stained with ProLong® Gold Antifade Mountant with DAPI. In the case of EMT-6 cells, micronuclei formation assays were performed at Phenovista Biosciences, San Diego. The cells were treated with either 1 μM or 5 μM SRA737 for 24h followed by mixing with 4% PFA for 20 minutes at RT directly in the culture wells, washing with PBS and staining with Hoechst (1:1000). The plates were imaged at 40× using the Thermo Fisher Scientific CellInsight CX7 imaging system and the micronuclei algorithm was adjusted to score all nuclei that are <55% of the size of the main nucleus. A total of 25 fields per well were imaged and the data was averaged across 8 replicates.

Statistics

Flow cytometry statistical analyses were performed with GraphPad Prism 5.0 software. Significant differences (p<0.05) between two groups were identified by Student's t-tests. The protein expression from cell lines was compared by t test between models sensitive to SRA737 (IC50<5 μM; n=13) and those that were resistant (IC50>5 μM; n=31). Proteins were selected with a greater than 2-fold change in IC50 values and p<0.05.

### Example 9: Human and Murine Cancer Cells Show Sensitivity to Chk1 Inhibition by SRA737

This example illustrates SRA737's ability to inhibit Chk1 in human and murine cancer cells.

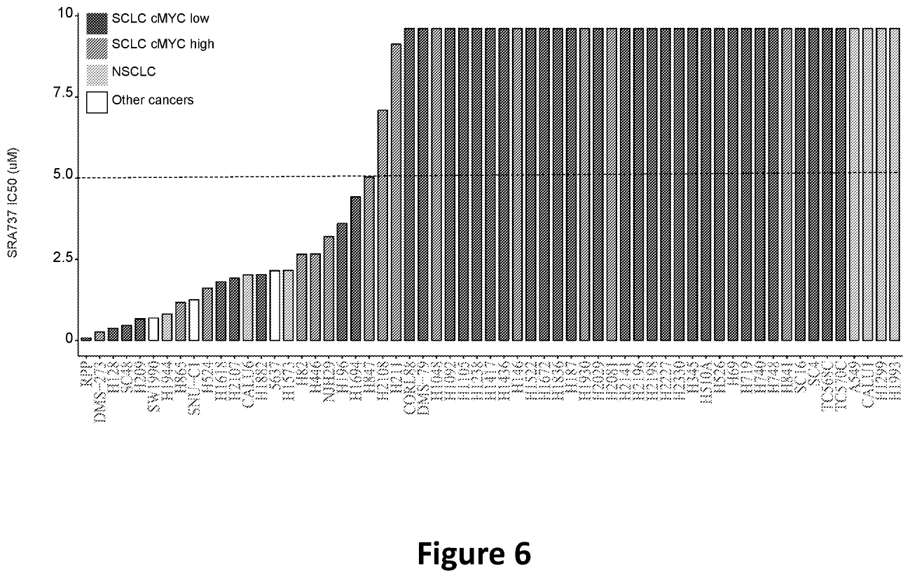

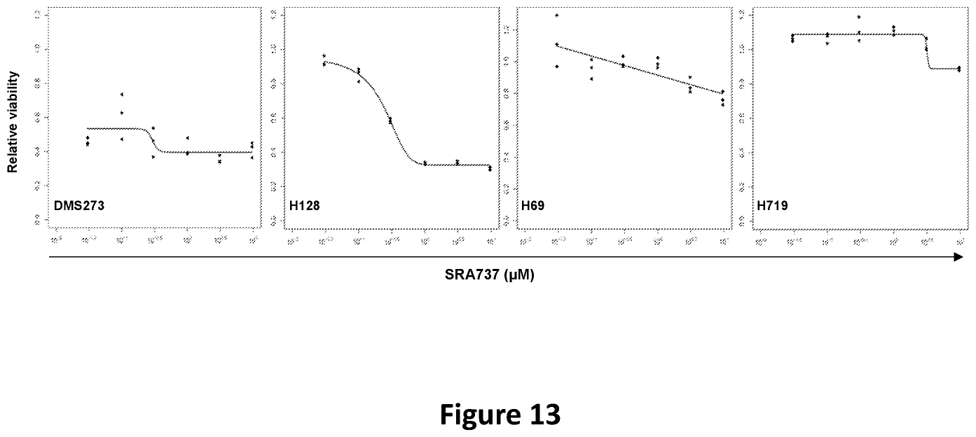

To investigate the in vitro activity of SRA737, a panel of 51 SCLC cell lines, including one murine derived model, and a panel of NSCLC, pancreatic, colon and bladder cancer cell lines were screened. The SCLC cell lines demonstrated a range of sensitivity to SRA737 and were classified according to the half-maximal inhibitory concentration (IC50) into sensitive (IC50<5 μM; n=15) and resistant (IC50>5 μM; n=35) (FIGS. 6 and 13).

In the other cancer types, a range of sensitivity was observed with low to sub-micromolar sensitivity, such as pancreatic cancer line SW1990 (IC50=0.7 uM), colon cancer line SNU-C1 (1.3 uM), bladder cancer line 5637 (IC50=2.1 uM) and NSCLC cancer lines (A549, Calu1, Calu6, H1299, H1573, H1944, H1993 IC50 range 0.8 uM to >9.6 uM) (FIG. 6).

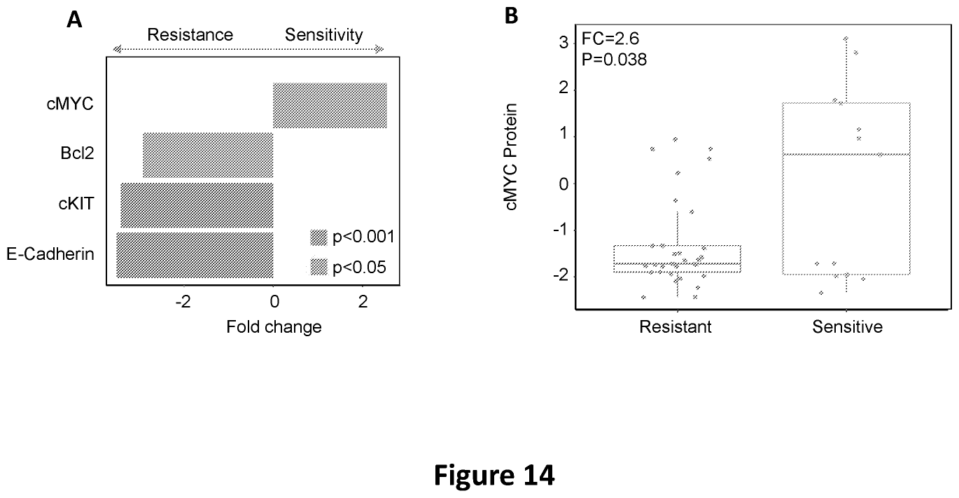

Having observed a range of in vitro responses to SRA737 in SCLC, identification of protein biomarkers of response was carried out next. Using available proteomic profiles as provided by reverse phase protein array (RPPA) from 44 of the cell lines, the analysis showed cMYC protein as a marker of SRA737 sensitive cell line and Bcl-2, cKit, and E-cadherin as markers of SRA737 resistant cell lines, as illustrated in FIG. 14.

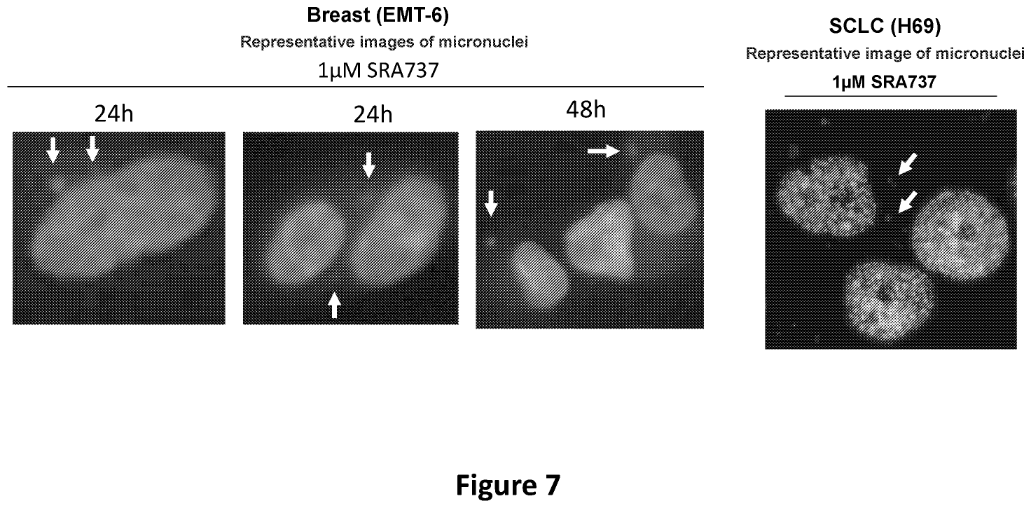

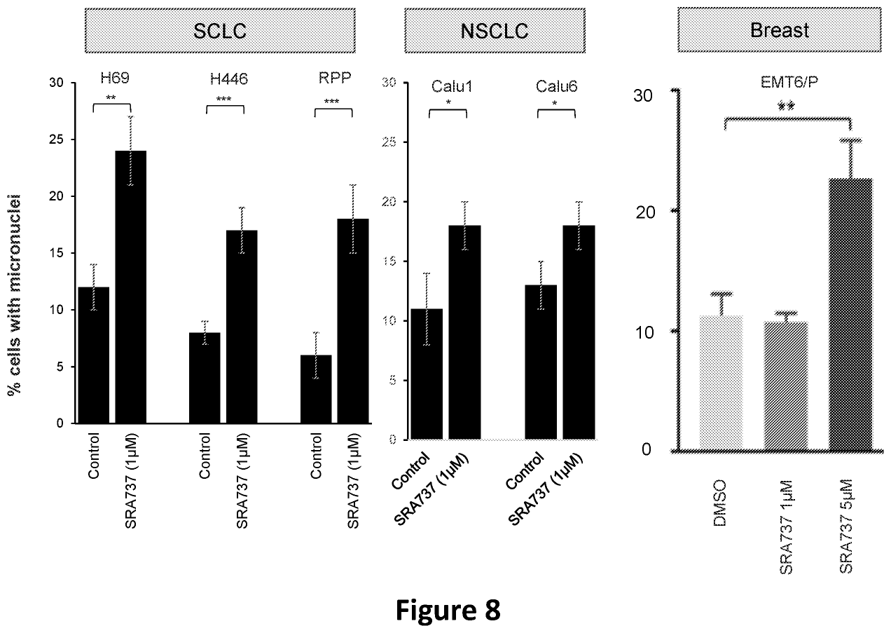

SRA737 was also evaluated for induced cytogenetic stress using a micronuclei (MN) assay. Treatment of SCLC cell line H69 and breast cancer cell line EMT6/P with SRA737 (1 μM) for 24 hours or 48 hours led to micronuclei formation, as shown in FIG. 7. The increase in MN frequency was quantified for EMT6/P cells and is shown in FIG. 8.

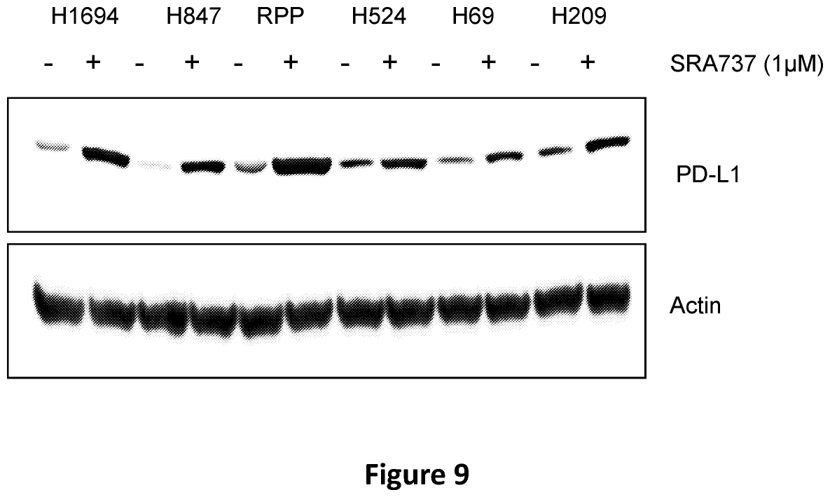

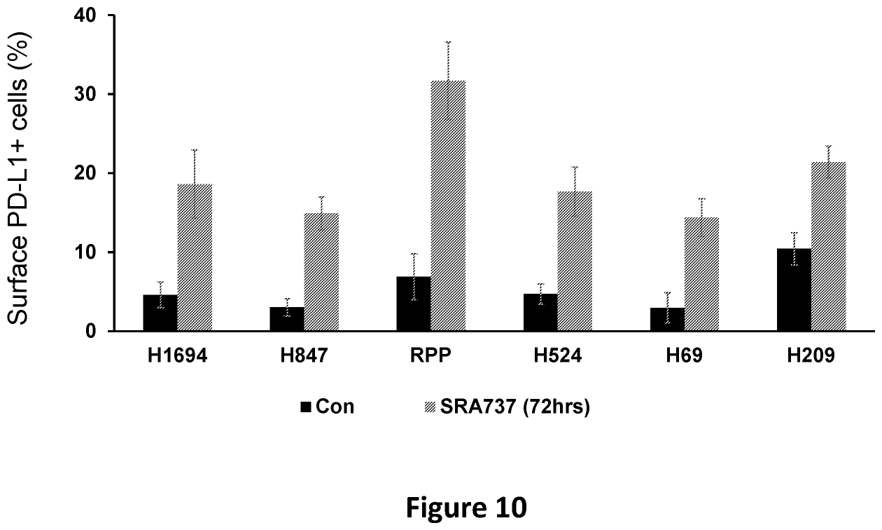

Furthermore, in all the SCLC cell lines tested, treatment with SRA737 (1 μM for 72 hours) significantly increased both the total PD-L1 levels and cell surface PD-L1 expression, as demonstrated in FIGS. 9 and 10 respectively.

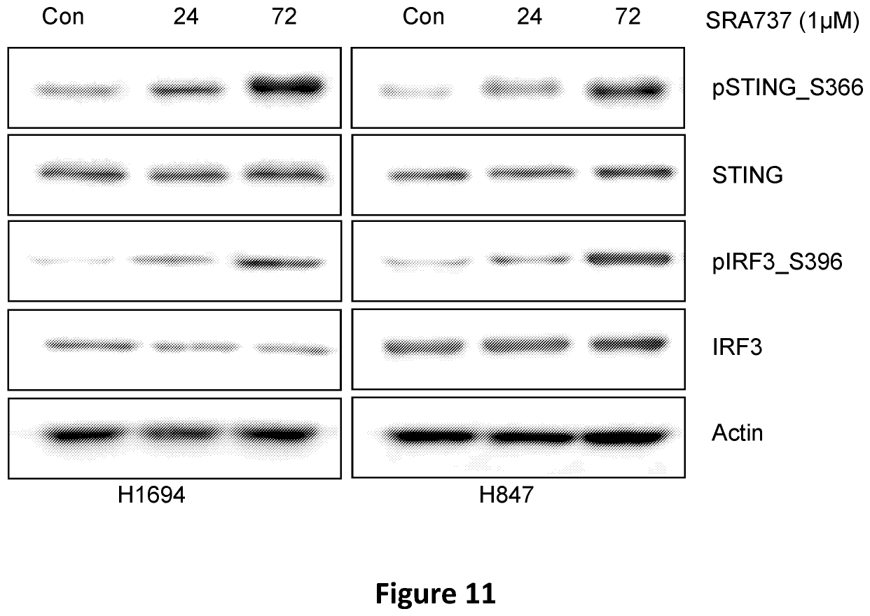

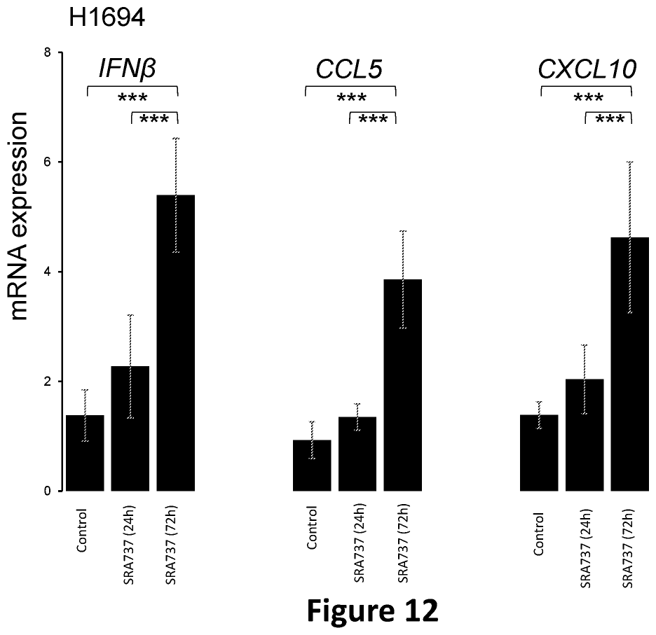

Activation of the STING pathway, namely pSTING_S366, STING, pIRF3_S396 and IRF3, in a time-dependent manner following SRA737 treatment in both H1694 and H847 cell lines was also demonstrated, as shown in FIG. 11. Targets of IRF3, including Type I interferon genes, particularly IFNβ, and downstream cytokines, such as CXCL10 and CCL5, were also activated by SRA737 treatment, as shown in FIG. 12. These results confirm that treatment with SRA737 has anti-proliferative and a potential immunomodulatory effect in multiple cancer models.

### Example 10: SRA737 Augments ICB-Induced Anti-Tumor Response in SCLC

This example demonstrates that SRA737 augmented an ICB-induced anti-tumor response in SCLC cells in mice.

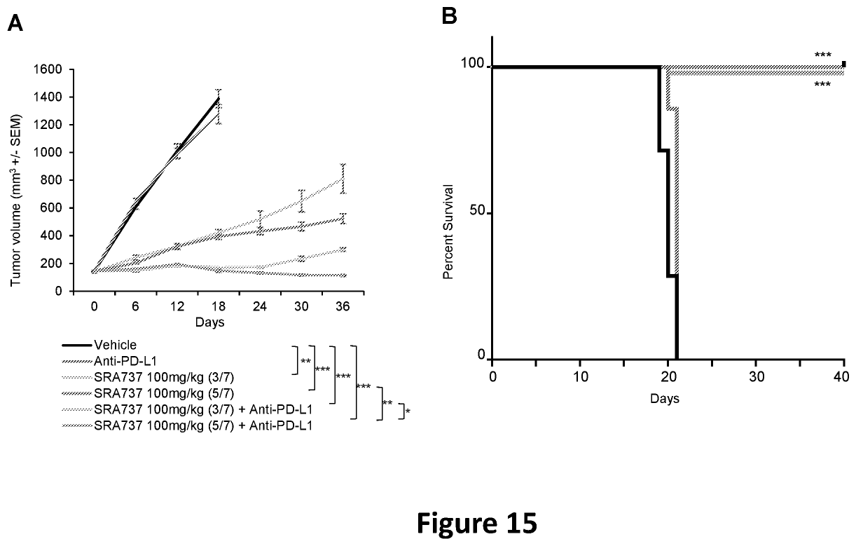

B6129F1 immunocompetent flank RPP tumor bearing mice were treated with two doses of SRA737 (100 mg/kg for 3-days-on/4-days-off and 100 mg/kg for 5-days-on/2-days-off) with and without anti-PD-L1 (300 μg, 1-day-on/6-days-off, n=10 per group) as shown in FIG. 15A. Mice treated with the anti-PD-L1 agent of B7-H1 alone showed no anti-tumor response in this model. Animals treated with a single agent of SRA737 showed significant reduction in tumor growth as compared to either the vehicle or the anti-PD-L1 treatment along, with the 5-days-on/2-days-off dosing regimen of SRA737 being moderately more effective than the 3-days-on/4-days-off dosing regimen.

Surprisingly, complete inhibition of tumor growth was observed in mice treated with the combination therapy comprising SRA737 and the anti-PD-L1 agent of B7-H1, as demonstrated in FIG. 13A. Similarly, the 5-days-on/2-days-off dosing regimen of SRA737 showed a better response when combined with the anti-PD-L1 agent of B7-H1 than the 3-days-on/4-days-off days dosing regimen with a significant survival benefit, as illustrated in FIG. 15B.

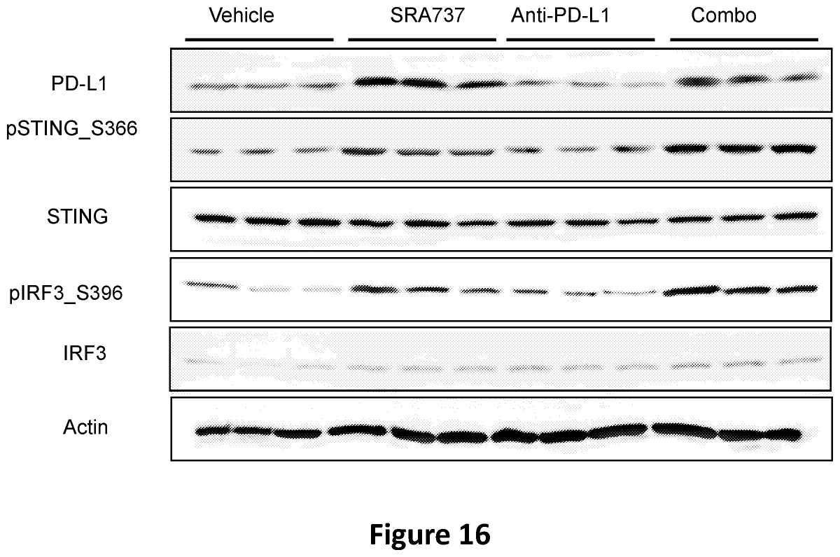

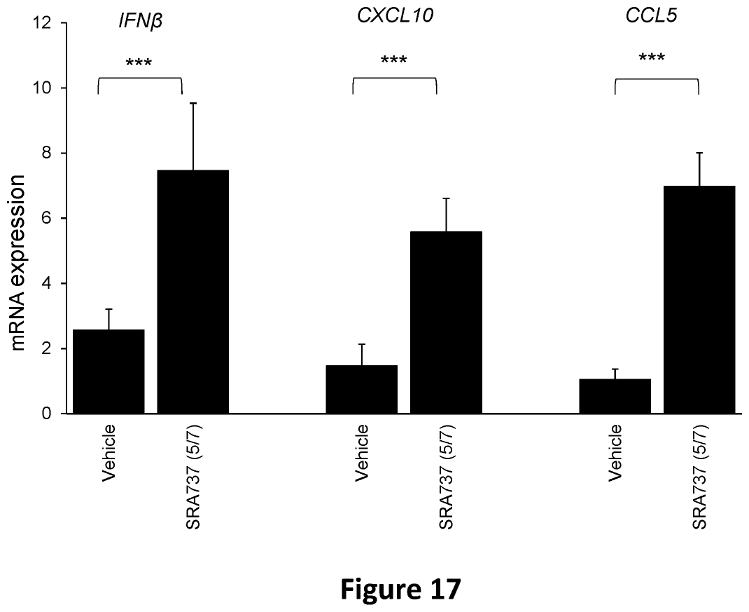

To further support that SRA737 activates the STING pathway in vitro as demonstrated in FIG. 11, tumor bearing mice were resected at Day 21 after treatment with SRA737 and/or the anti-PD-L1 agent of B7-H1. Immunoblot analysis showed treatment with both SRA737 alone and in combination with the anti-PD-L1 agent of B7-H1 led to significant activation of the STING pathway including pSTING_S366, pTBK1_S172, cGAS, and pIRF3_S396, as shown in FIG. 16. Furthermore, higher gene expression levels of IFNβ, CXCL10 and CCL5 post treatment with SRA737 (100 mg/kg for 5-days-on/2-days-off) as compared to vehicle in the same SCLC tumors at Day 21 were observed, as shown in FIG. 17. These results demonstrated that the combination of SRA737 and ICB provided strong anti-tumor activity in SCLC. Furthermore, treatments with both single agent SRA737 and its combination with the anti-PD-L1 agent of B7-H1 led to activation of the STING pathway and chemokine expression in this SCLC model.

### Example 11: SRA737 Augments ICB-Induced Anti-Tumor Response in Multiple Cancers

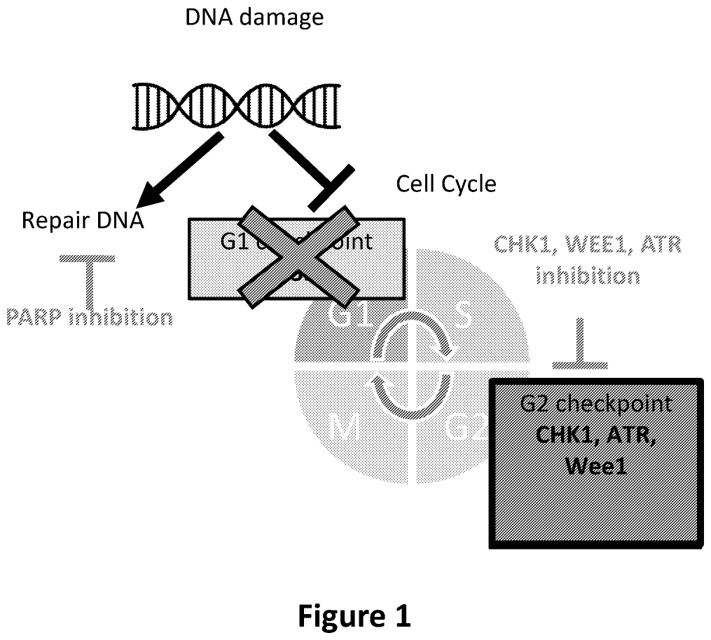

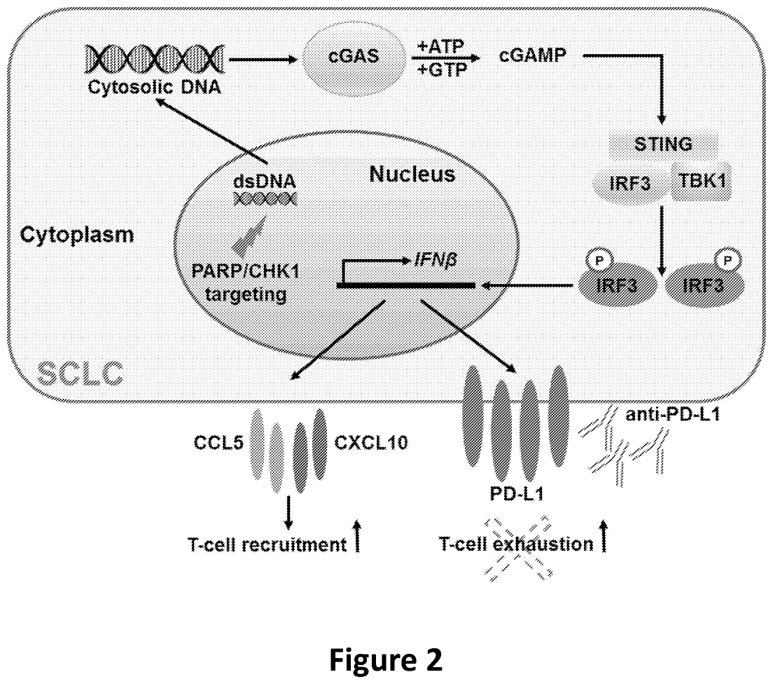

This example demonstrates the syngeneic anti-tumor response of the combination therapy representing other cancer indications included in FIG. 1.

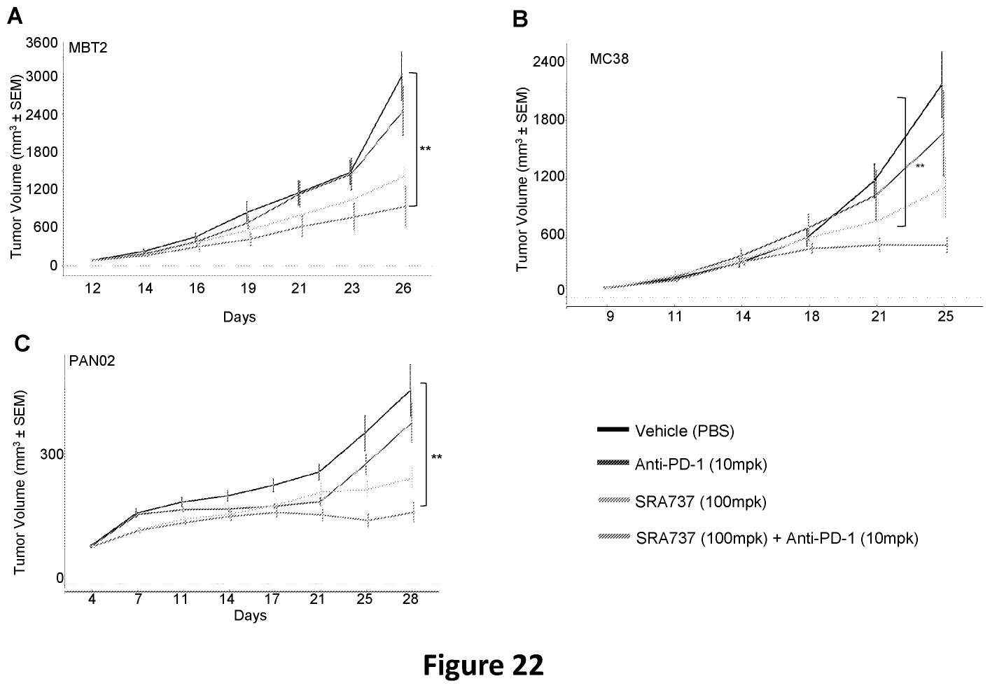

Tumors cells were implanted into the flank of immunocompetent mice. Once the volumes of the tumor reached 150-200 mm³, the mice were randomized and treated with either monotherapy of SRA737 at 100 mg/kg for 5-days-on/2-days-off p.o., anti-PD1 BIW at 10 mg/kg i.p. or the combination thereof. Similar to anti-PD-L1 treatment in the SCLC model, monotherapy treatment with anti-PD1 showed very limited activity in the colon (MC38−TGI=23%), bladder (MBT-2−TGI=18%) and pancreatic (Pan02−TGI=25%) models, while SRA737 monotherapy (100 mg/kg, 5/7) induced moderate tumor growth inhibition in all models, namely TGI-MC-38 (48%), MBT-2 (53%), and Pan02 (59%). However, combination treatment of SRA737 and anti-PD1 resulted in substantially improved efficacy in all three models, namely TGI-MC38 (76%) as shown in FIG. 22A, MBT-2 (69%) as shown in FIG. 22B, and Pan02 (70%) as shown in FIG. 22C.

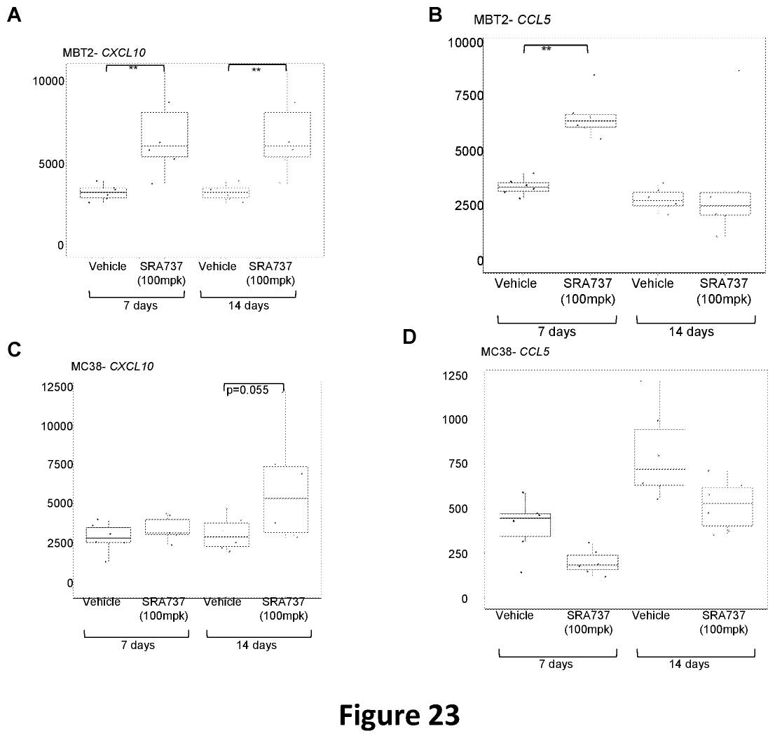

The flank of immunocompetent mice was then treated with SRA737 monotherapy for 5-days-on/2-days-off for either one or two cycles and the expression of CCL5 and CXCL10 in extracted tumors were analyzed. In MBT-2 tumors, CXCL10 showed a significant induction after both 7 days and 14 days of SRA737 treatment (p<0.05), as shown in FIG. 23A. Similarly, the levels of CCL5 were also significantly induced after 7 days of treatment (p<0.01), while no significant difference after 14 days was observed, as shown in FIG. 23B. In the MC38 model, CXCL10 levels were significantly increased (p<0.05) after 14 days of treatment (FIG. 23C), while expression of CCL5 was modestly but not statistically significantly reduced after 7 days (FIG. 23D). Based on these results, the MC38 model was chosen since it showed the best efficacy with the combination treatment of SRA737 and anti-PD1. The previously used dosing regimen of 5-days-on/2-days-off was modified to a schedule of 7-days-on/7-days-off for SRA737 treatment with or without co-treatment with anti-PD1 (Days 3, 7 and 11) based on the results of induction of chemokines secretion after 7 days. This data supported the theory that the induction of interferon response genes, such as chemokines CCL5 and CXCL10, would result in the recruitment of immune cells into the tumors.

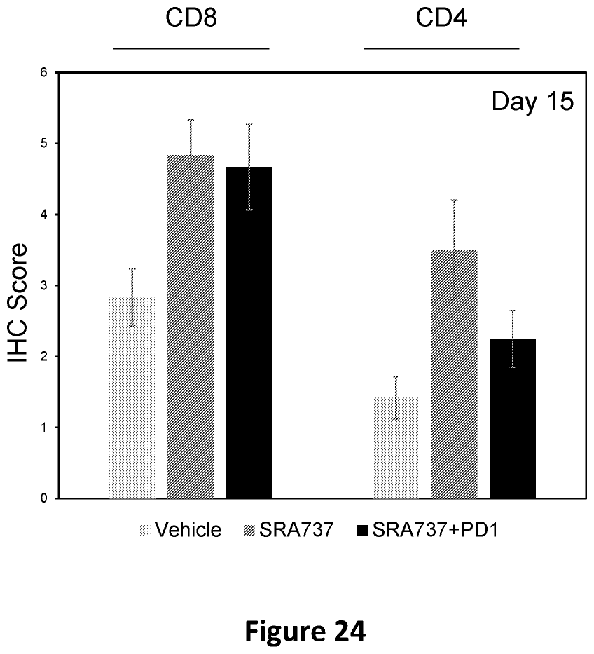

The MC38 tumors were then harvested after 14 days of treatment with SRA737 monotherapy and its combination therapy with an anti-PD-L1 agent (Days 3, 7 and 11) according to a modified dosing regimen of 7-days-on/7-days-off to determine the infiltration of CD4+ and CD8+ cells by immunohistochemistry staining. It was demonstrated and as shown in FIG. 24 that both SRA737 monotherapy and its combination therapy with the anti-PD-L1 agent of B7-H1 induced a significant increase in the infiltration of CD4+ and CD8+ cells into the tumors. The tumor growth inhibition under this altered dosing regimen was 61% in SRA737 monotherapy and 63% in the combination therapy of SRA737 and anti-PD-1. This observed difference in the efficacy of tumor growth inhibition could have been caused by the altered SRA737 dosing regimen and that it could reflect a dual action of SRA737, namely a direct anti-tumor activity and a modulation of the infiltration and the proliferation of the immune cells in the tumors. Thus, these results highlighted the need to optimize the dosing regimen of SRA737 in a way that will maximize the tumor cell killing potential of SRA737 through a combination of the two mechanisms to avoid immunosuppression.

### Example 12: Combination of SRA737 and Gemcitabine Chemotherapy Enhances the Effect of Anti-PD-L1 Therapy by Modulating the Immune Microenvironment

This example illustrates the synergistic effect of the triple combination therapy of SRA737, low-dose gemcitabine, and the anti-PD-L1 agent of B7-H1 in inducing tumor regression by modulating the immune microenvironment.

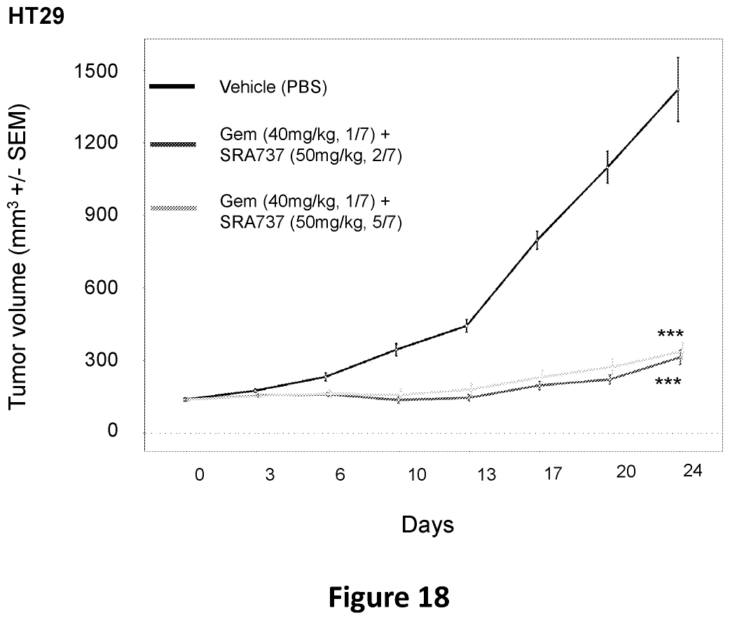

The combination therapy of SRA737 and LDG gave very similar anti-tumor activity in the colon cancer HT29 in vivo model regardless of dosing regimen for SRA737 (2-days-on/5-days-off or 5-days-on/2-days-off in a weekly cycle) as shown in FIG. 18. Thus, the dosing regimen of 2-days-on/5-days-off for SRA737 was chosen for studies in the SCLC model to avoid immunosuppression.

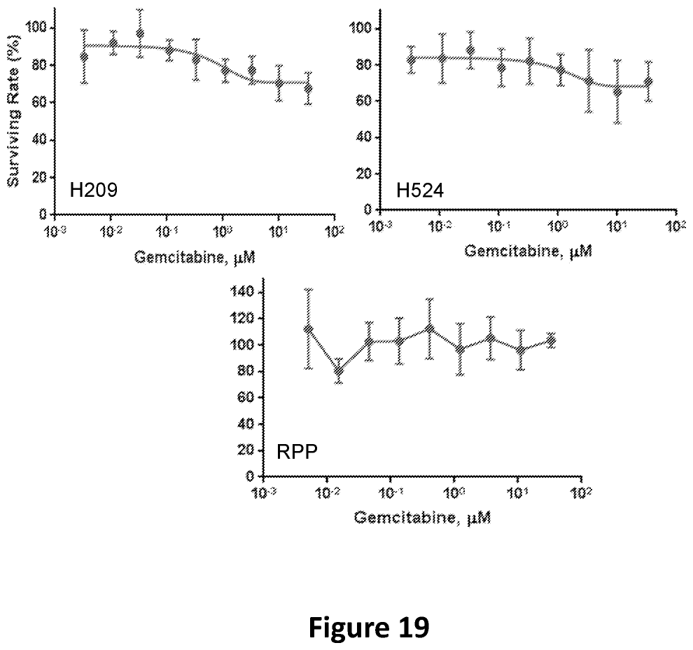

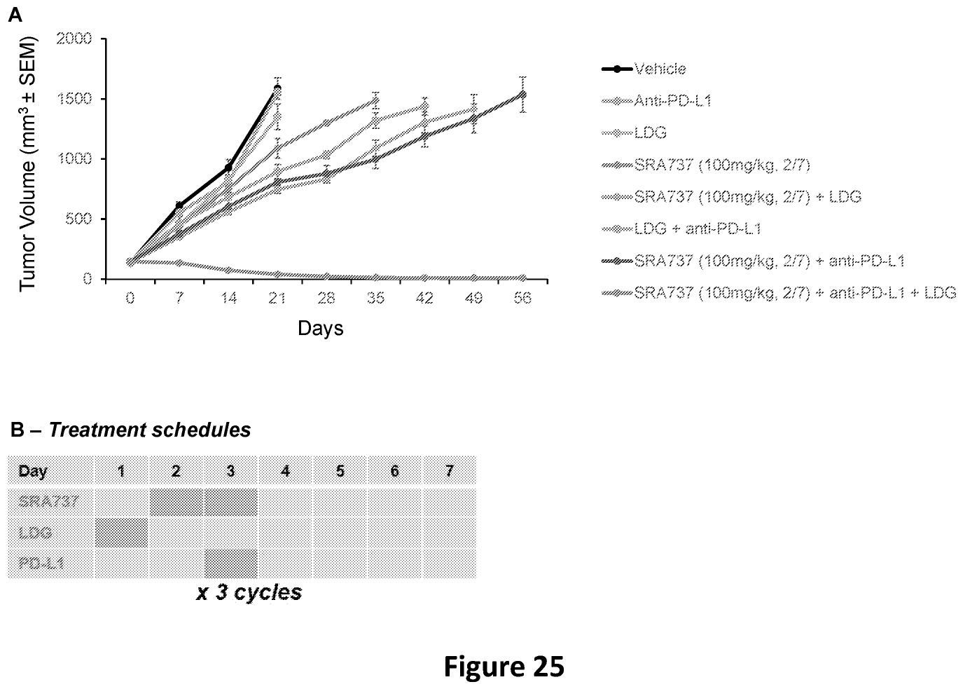

FIG. 19 shows that cancer cells in the SCLC in vitro models of H209, H524 and RPP were resistant to gemcitabine alone. Furthermore, when RPP tumor-bearing B6129F1 immunocompetent mice were treated with monotherapies of anti-PD-L1 at 300 μg (i-day-on/6-days-off), LDG at 40 mg/kg (1-day-on/6-days-off) or SRA737 at 100 mg/kg (2-day-on/5-days-off) separately, no significant anti-tumor effect was observed with these single agent treatments (FIG. 25A). The combination therapy of SRA737 and anti-PD-L1 agent also gave only a moderate delay in tumor growth. However, a remarkably synergistic effect in sustained tumor regression, as shown in FIG. 25A, was observed when the triple combination therapy comprising LDG, SRA737, and the anti-PD-L1 agent of B7-H1 were administered to RPP tumor-bearing mice according to the dosing regimen illustrated in FIG. 25B. Surprisingly, all mice had tumor regression with 80% of them showing sustained tumor clearance of up to 60 days after treatment as shown in FIG. 25A.

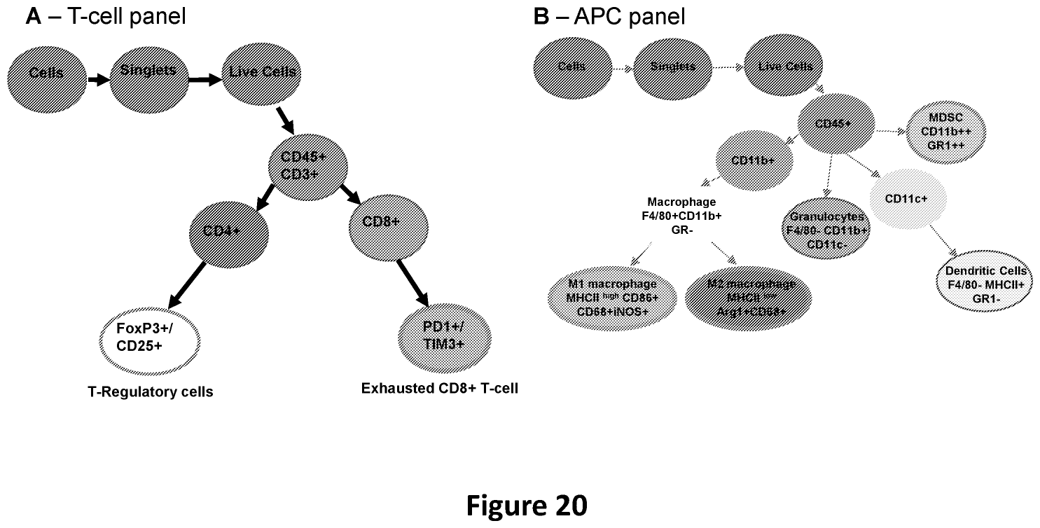

Although PD-L1 blockade has been shown to active T-cells in SCLC, the role of this pathway on tumor-associated macrophages (TAMs) is not well known. Thus, a cohort of tumors (n=8) were resected at Day 21 of the triple combination treatment and analyzed by multicolor flow cytometry for the changes in tumor infiltrating lymphocytes, or the T-cell panel as shown in FIG. 20A. Also, the role of antigen presenting cells, or the APC panel as illustrated in FIG. 20B, that include macrophages, dendritic cells and granulocytes were also evaluated in the SCLC model with the triple combination therapy of LDG, SRA737, and anti-PD-L1 according to the dosing regimen outlined in FIG. 25B.

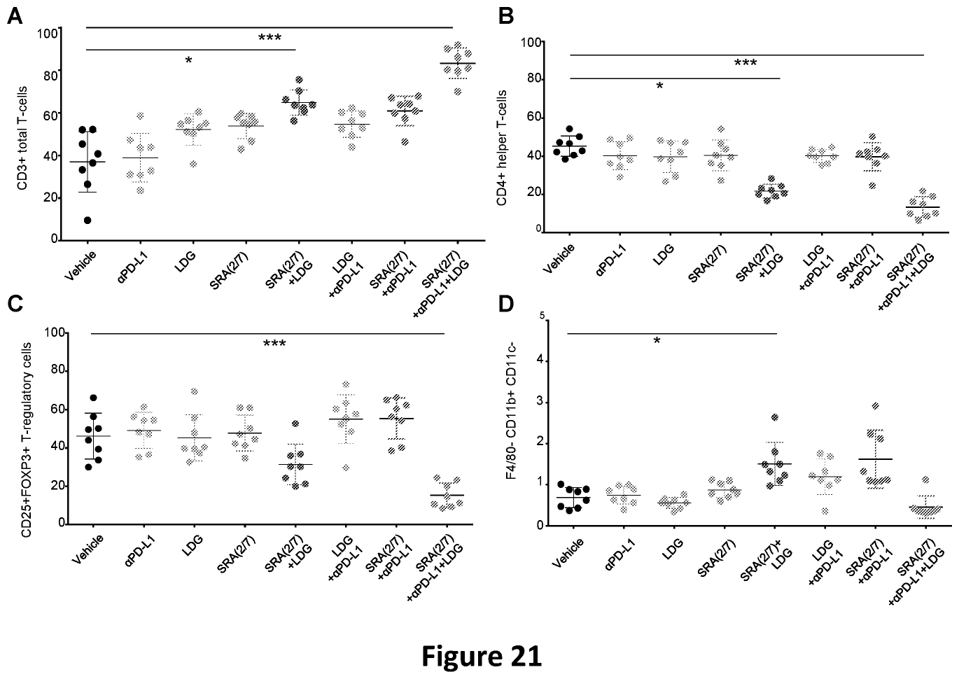

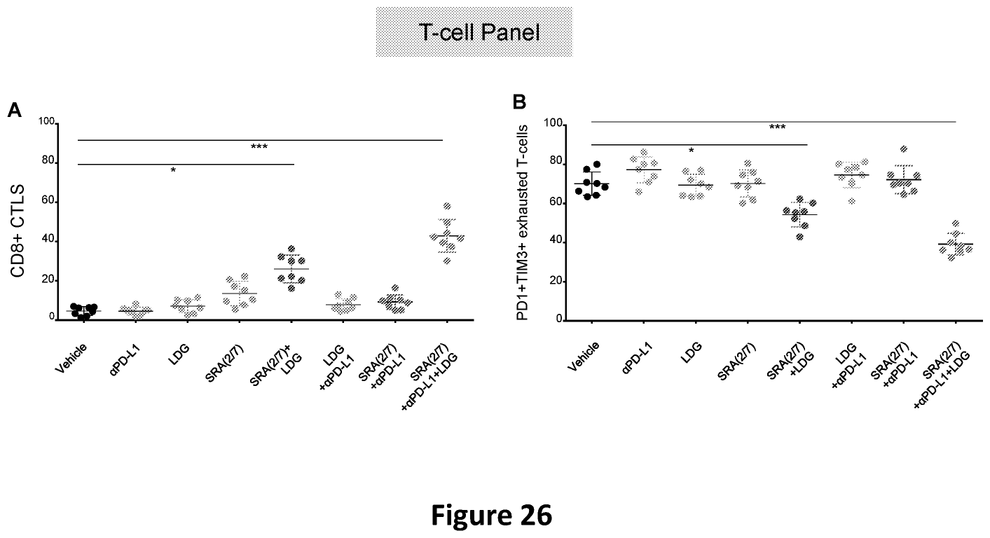

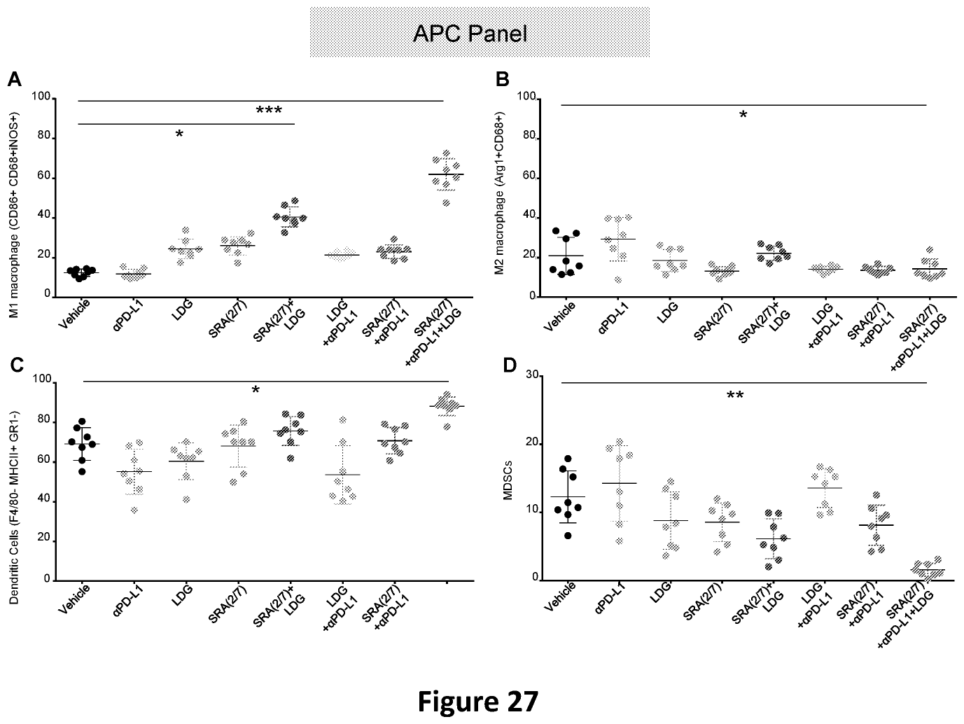

Both the combination treatment of LDG and SRA737 and the triple combination treatment of LDG, SRA737 and anti-PD-L1 significantly increased CD3+(FIG. 21A) and CD8+(FIG. 26A) total T-cell infiltration (p<0.001 for both). Additionally, the triple combination treatment resulted in significant decreases in the infiltrations of CD4+ helper T-cells (FIG. 21B), CD25+/FOXP3+ regulatory T-cells (FIG. 21C), and PD1+/TIM3+ exhausted CD8+ T cell population (FIG. 26B) with p<0.001 for all. Interestingly, in the APC panel, the triple combination led to a significant increase in the M1 macrophage population with a decrease in the M2 macrophage population, as shown in FIGS. 27A and 27B respectively. Furthermore, an increase of dendritic cells (FIG. 27C) and a decrease of MDSC immune subset (FIG. 27D) were observed post treatment of the triple combination comprising of LDG, SRA737, and anti-PD-L1, but no significant change in the granulocyte population was observed in any treatment groups as shown in FIG. 21D.

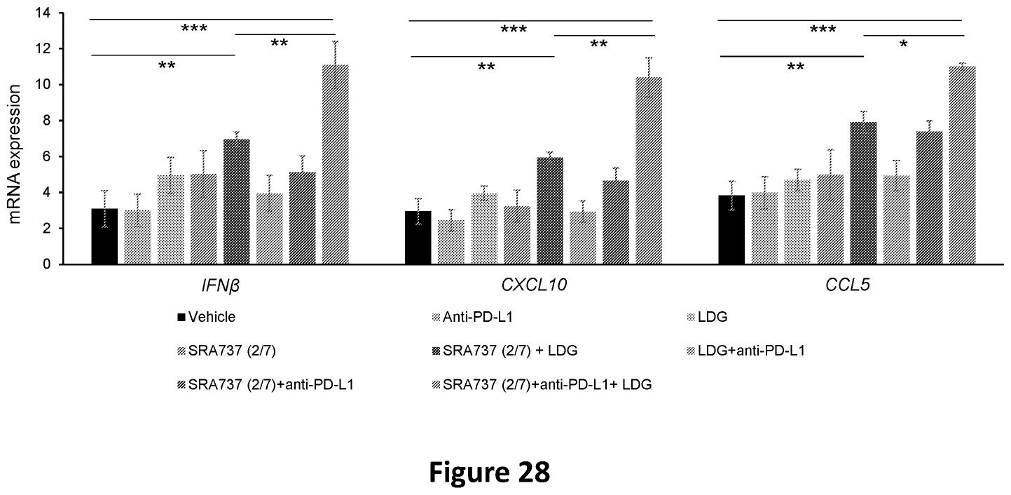

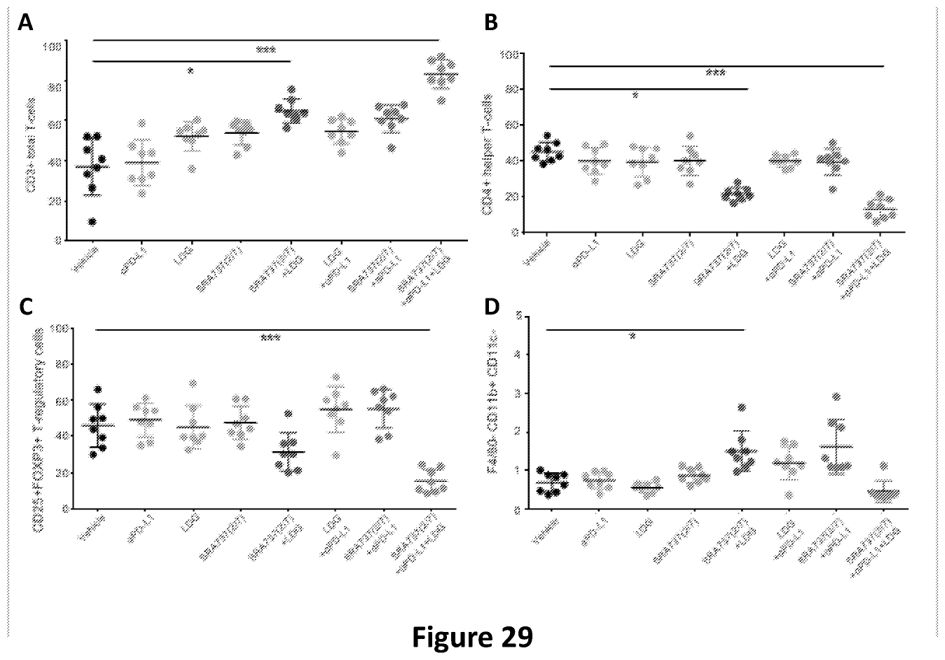

Lastly, the effect of combination therapies (LDG plus SRA737, SRA737 plus anti-PD-L1, or LDG and SRA737plus anti-PD-L1) were investigated to better understand the immune microenvironment in SCLC. Real-time quantitative PCR analyses of IFNβ, CXCL10 and CCL5 in resected tumors at Day 21 of treatment were carried out. Neither LDG nor SRA737 monotherapy (2-days-on/5-days-off) showed any significant effect on Type I interferon gene of IFNβ, or the chemokines of CCL5 or CXCL10, as demonstrated in FIG. 28. While the combination therapies of LDG plus SRA737 and SRA737 plus anti-PD-L1 led to only modest enhancement of IFNβ, CCL5 or CXCL10 expressions at best, the triple combination therapy comprising of LDG, SRA737 and anti-PD-L1 treatments led to significantly enhanced expressions of IFNβ, CXCL10 and CCL5 in SCLC tumors, as illustrated in FIG. 28.

In summary, it has been clearly demonstrated that the triple combination therapy of LDG, SRA737 and the anti-PD-L1 agent of B7-H1 provided strong synergistic effect in inducing dramatic anti-tumor regressive activity and established a significant immune microenvironment in SCLC models in mice.

1. A method of treating cancer, comprising:
administering to a subject with cancer at least one treatment cycle, the treatment cycle comprising administering effective amounts of a chemotherapeutic agent, a checkpoint kinase 1 (Chk1) inhibitor, and an immune checkpoint inhibitor.

2. The method of claim 1, wherein the chemotherapeutic agent is selected from the group consisting of olaparib, niraparib, rucaparib, gemcitabine, talazoparib, cisplatin, a ribonucleotide reductase inhibitor, etoposide, SN-38/CPT-11, mitomycin C, and combinations thereof.

3. The method of claim 1 or 2, wherein the immune checkpoint inhibitor is selected from the group consisting of an anti-CTLA-4 antibody, an anti-PD-L1 antibody, an anti-PD-1 antibody, an IDO1 inhibitor, and combinations thereof.

4. The method of any one of claims 1-3, wherein the immune checkpoint inhibitor is selected from the group consisting of ipilimumab, pembrolizumab, nivolumab, atezolizumab, durvalumab, avelumab, epacadostat, indoximod, cemiplimab, F-001287, and NLG919.

5. The method of claim 3, wherein the immune checkpoint inhibitor is an anti-PD-L1 antibody and is selected from the group consisting of atezolizumab, avelumab, and durvalumab.

6. The method of claim 3, wherein the immune checkpoint inhibitor is an anti-PD-1 antibody and is selected from the group consisting of nivolumab, pembrolizumab, and cemiplimab.

7. The method of any one of claims 1-6, wherein the Chk1 inhibitor is SRA737.

8. The method of any one of claims 1-7, wherein the routes of administration of the chemotherapeutic agent, checkpoint kinase 1 (Chk1) inhibitor, and immune checkpoint are independently selected from the group consisting of intravenous, subcutaneous, cutaneous, oral, intramuscular, and intraperitoneal.

9. The method of any one of claims 1-8, wherein the cancer is selected from the group consisting of bladder cancer, breast cancer, cervical cancer, colorectal cancer, esophageal cancer, gastric cancer, head and neck cancer, hepatocellular cancer, leukemia, lung cancer, lymphoma, mesothelioma, melanoma, myeloma, ovarian cancer, high-grade serious ovarian cancer, endometrial cancer, prostate cancer, pancreatic cancer, renal cell cancer, non-small cell lung cancer (NSCLC), small cell lung cancer (SCLC), brain cancer, sarcoma, neuroblastoma, anal cancer, squamous cell carcinoma of anus, penile squamous cell carcinoma, vulvar squamous cell carcinoma, squamous cell carcinoma of cervical, or squamous cell carcinoma of the head and neck cancerous cells.

10. The method of any one of claims 1-9, wherein growth of a tumor is reduced in the subject after administration.

11. The method of any one of claims 1-10, wherein tumor growth in the subject is reduced by at least 1% after administration.

12. The method of any one of claims 1-10, wherein tumor growth in the subject is reduced by at least 5% after administration.

13. The method of any one of claims 1-10, wherein tumor growth in the subject is reduced by at least 10% after administration.

14. The method of any one of claims 1-10, wherein tumor growth in the subject is reduced by at least 20% after administration.

15. The method of any one of claims 1-10, wherein tumor growth in the subject is reduced by at least 30% after administration.

16. The method of any one of claims 1-10, wherein tumor growth in the subject is reduced by at least 40% after administration.

17. The method of any one of claims 1-10, wherein tumor growth in the subject is reduced by at least 50% after administration.

18. The method of any one of claims 1-10, wherein tumor growth in the subject is reduced by at least 60% after administration.

19. The method of any one of claims 1-10, wherein tumor growth in the subject is reduced by at least 70% after administration.

20. The method of any one of claims 1-10, wherein tumor growth in the subject is reduced by at least 80% after administration.

21. The method of any one of claims 1-10, wherein tumor growth in the subject is reduced by at least 90% after administration.

22. The method of any one of claims 1-10, wherein tumor growth in the subject is reduced by at least 95% after administration.

23. The method of any one of claims 1-10, wherein tumor growth in the subject is reduced by at least 99% after administration.

24. The method of any one of claims 1-23, wherein effective amounts of gemcitabine, SRA737, and an immune checkpoint inhibitor are administered at the same time.

25. The method of any one of claims 1-23, wherein effective amounts of gemcitabine, SRA737, and an immune checkpoint inhibitor are administered separately.

26. The method of any one of claims 1-23, wherein the effective amounts of gemcitabine, SRA737, and an immune checkpoint inhibitor are administered sequentially.

27. The method of any one of claims 1-26, wherein the method comprises administering a first effective amount of gemcitabine, a first effective amount of SRA737, a second effective amount of SRA737, and a first effective amount of an immune checkpoint inhibitor.

28. The method of any one of claims 1-27, wherein the first effective amount of gemcitabine is administered before the administrations of the first and second effective amounts of SRA737 and the FIRST effective amount of an immune checkpoint inhibitor.

29. The method of any one of claims 1-28, wherein the first effective amount of gemcitabine is administered 12-24 hours before the administrations of the first and second effective amounts of SRA737 and the first effective amount of an immune checkpoint inhibitor.

30. The method of any one of claims 1-29, wherein the first effective amount of SRA737 is administered 12-24 hours after the administration of the first effective amount of gemcitabine and before the administrations of the second effective amount of SRA737 and the first effective amount of an immune checkpoint inhibitor.

31. The method of any one of claims 1-30, wherein the second effective amount of SRA737 is administered 12-48 hours after the administration of the first effective amount of gemcitabine and first effective amount of SRA737 and 12-24 hours before the administration of the first effective amount of an immune checkpoint inhibitor.

32. The method of any one of claims 1-31, wherein the second effective amount of SRA737 is administered at the same time as the first effective amount of an immune checkpoint inhibitor and 12-48 hours after the administrations of the first effective amount of gemcitabine and the first effective amount of SRA737.

33. The method of any one of claims 1-32, wherein the entire dosage of at least one of, or all of, or any combination of, gemcitabine, Chk1 inhibitor, and/or an immune checkpoint inhibitor is administered in a single administration.

34. The method of any one of claims 1-32, wherein the administration of the entire dosage of at least one of, or all of, or any combination thereof gemcitabine, Chk1 inhibitor, and/or an immune checkpoint inhibitor proceeds continuously over a period of 0-12 hours.

35. The method of any one of claims 1-34, wherein the first effective amount of SRA737 is 0.001 mg/day to 1000 mg/day and the second effective amount is 0.001 mg/day to 1000 mg/day.

36. The method of any one of claims 1-35, wherein the treatment cycle is repeated at least one time.

37. The method of any one of claims 1-36, wherein the treatment cycle is repeated at least three times.

38. The method of claim 36 or 37, wherein the treatment cycle comprises a time period of at least four days.

39. The method of any one of claims 1-38, wherein the cancer is SCLC.

40. The method of any one of claims 1-39, wherein the method comprising administering an effective amount of SRA737, wherein the effective amount is less than 2000 mg/day.

41. The method of claim 40, wherein the SRA737 compound is administered orally.

42. The method of claim 40 or 41, wherein the SRA737 compound is administered daily.

43. The method of claim 42, wherein the SRA737 compound is administered for at least 28 consecutive days.

44. The method of claim 42, wherein the SRA737 compound is administered for at least 7 consecutive days.

45. The method of claim 40 or 41, wherein the SRA737 compound is administered intermittently.

46. The method of claim 33, wherein the SRA737 compound is administered with at least ten (10) minutes, fifteen (15) minutes, twenty (20) minutes, thirty (30) minutes, forty (40) minutes, sixty (60) minutes, two (2) hours, three (3) hour, four (4) hours, six (6) hours, eight (8) hours, ten (10) hours, twelve (12) hours, fourteen (14) hours, eighteen (18) hours, twenty-four (24) hours, thirty-six (36) hours, forty-eight (48) hours, three (3) days, four (4) days, five (5) days, six (6) days, seven (7) days, eight (8) days, nine (9) days, ten (10) days, eleven (11) days, twelve (12) days, thirteen (13) days, fourteen (14) days, three (3) weeks, or four (4) weeks, delay between administrations.

47. The method of any one of claims 40-46, wherein the SRA737 compound is administered over one or more 28 day cycles.

48. The method of claim 47, wherein the SRA737 compound is administered on one or more days of the one or more 28 day cycles.

49. The method of any one of claim 48, wherein the SRA737 compound is administered on days 2, 3, 9, 10, 16, and 17 of the one or more 28 day cycles.

50. The method of any one of claims 47-49, further comprising administering an initial dose of the SRA737 compound prior to the first of the one or more 28 day cycles.

51. The method of claim 50, wherein the initial dose is administered 4 days, 5 days, 6 days, or 7 days prior to the first cycle of the one or more 28 day cycles.

52. The method of any one of claims 47-51, wherein the one or more 28 day cycles comprises 2, 3, 4, 5, 6 or more 28 day cycles.

53. The method of any one of claims 40-46, wherein the SRA737 compound is administered following a dosing schedule selected from the group consisting of 5 days of dosing followed by 2 days of non-dosing each week, 1 week of daily dosing followed by 1, 2, or 3 weeks of non-dosing, 2 or 3 weeks of daily dosing followed by 1 or 2 weeks of non-dosing, and dosing on days 2 and 3 of a weekly cycle.

54. The method of any one of claims 40-53, wherein the effective amount is administered in a single dose once a day.

55. The method of any of claims 40-5353, wherein half of the effective amount is administered twice a day.

56. The method of any of claims 40-55, wherein the effective amount is less than 1500 mg/day.

57. The method of any of claims 40-55, wherein the effective amount is less than 1300 mg/day.

58. The method of any of claims 40-55, wherein the effective amount is 1000 mg/day or less.

59. The method of any of claims 40-55, wherein the effective amount is 900 mg/day or less.

60. The method of any of claims 40-55, wherein the effective amount is 800 mg/day or less.

61. The method of any of claims 40-55, wherein the effective amount is 700 mg/day or less.

62. The method of any of claims 40-55, wherein the effective amount is 600 mg/day or less.

63. The method of any of claims 40-55, wherein the effective amount is 500 mg/day or less.

64. The method of any of claims 40-55, wherein the effective amount is 400 mg/day or less.

65. The method of any of claims 40-55, wherein the effective amount is between 600 mg/day and 1300 mg/day.

66. The method of any of claims 40-55, wherein the effective amount is between 300 mg/day and 1300 mg/day.

67. The method of any of claims 40-55, wherein the effective amount is between 300 mg/day and 1000 mg/day.

68. The method of any of claims 40-55, wherein the effective amount is between 300 mg/day and 800 mg/day.

69. The method of any of claims 40-55, wherein the effective amount is between 500 mg/day and 1300 mg/day.

70. The method of any of claims 40-55, wherein the effective amount is between 500 mg/day and 1000 mg/day.

71. The method of any of claims 40-55, wherein the effective amount is between 500 mg/day and 800 mg/day.

72. The method of any of claims 40-55, wherein the effective amount is selected from the group consisting of: 600 mg/day, 700 mg/day, 800 mg/day, 900 mg/day, 1000 mg/day, 1100 mg/day, and 1200 mg/day.

73. The method of any of claims 40-55, wherein the effective amount is selected from the group consisting of: 40 mg/day, 80 mg/day, 300 mg/day, 500 mg/day, 600 mg/day, 700 mg/day, and 800 mg/day.

74. The method of any of claims 40-55, wherein the effective amount is 300 mg/day.

75. The method of any of claims 40-55, wherein the effective amount is 400 mg/day.

76. The method of any of claims 40-55, wherein the effective amount is 500 mg/day.

77. The method of any of claims 40-55, wherein the effective amount is 600 mg/day.

78. The method of any of claims 40-55, wherein the effective amount is 700 mg/day.

79. The method of any of claims 40-55, wherein the effective amount is 800 mg/day.

80. The method of any of claims 40-55, wherein the effective amount is 900 mg/day.

81. The method of any of claims 40-55, wherein the effective amount is 1000 mg/day.

82. The method of any of claims 1-81, wherein the cancer is metastatic cancer.

83. The method of claim 1-81, wherein the cancer is colorectal cancer.

84. The method of claim 83, wherein the colorectal cancer is characterized as having a microsatellite instability or a deficiency in mismatch repair (MMR).

85. The method of claim 1-81, wherein the cancer is non-small cell lung cancer.

86. The method of claim 1-81, wherein the cancer is HNSCC.

87. The method of claim 1-81, wherein the cancer is SCCA.

88. The method of claim 1-81, wherein the cancer is anogenital cancer.

89. The method of claim 1-81, wherein the cancer is prostate cancer.

90. The method of claim 89, wherein the prostate cancer is metastatic castration-resistant prostate cancer (mCRPC).

91. The method of claim 1-81, wherein the cancer is ovarian cancer.

92. The method of claim 91, wherein the ovarian cancer is high-grade serous ovarian cancer (HGSOC).

93. The method of claim 92, wherein a tumor associated with the HGSOC is identified as having an increased expression of a Cyclin E1 (CCNE) gene.

94. The method of claim 93, wherein the increased expression is a result of genetic amplification.

95. The method of claim 92, wherein the tumor is identified as having somatic or germline BRCA1 and BRCA2 wild-type status.

96. The method of any of claims 1-95, wherein a tumor associated with the cancer is identified as having a gain of function mutation, amplification or overexpression of at least one oncogenic driver gene or other gene implicated in Chk1 pathway sensitivity.

97. The method of claim 96, wherein the oncogenic driver gene is selected from the group consisting of: MYC, MYCN, KRAS, and CCNE1.

98. The method of any of claims 1-97, wherein a tumor associated with the cancer is identified as having a loss of function or a deleterious mutation in at least one DNA damage repair (DDR) pathway gene implicated in Chk1 pathway sensitivity.

99. The method of claim 98, wherein the DDR pathway gene is selected from the group consisting of: ATM, CDK12, RAD51C. BRCA1, BRCA2, MRE11A, ATR, and an FA pathway gene.

100. The method of claim 98 or 99 wherein the loss of function or the deleterious mutation is determined by establishing microsatellite instability or a deficiency in mismatch repair (MMR).

101. The method of any of claims 1-100, wherein a tumor associated with the cancer is identified as having a gain of function mutation or amplification of at least one replication stress gene implicated in Chk1 pathway sensitivity.

102. The method of claim 101, wherein the replication stress gene is ATR or Chk1.

103. The method of any of claims 1-102, wherein a tumor associated with the cancer is identified as having a deleterious mutation in a tumor suppressor (TS) gene implicated in Chk1 pathway sensitivity.

104. The method of claim 103, wherein a tumor associated with the cancer suppressor gene is selected from the group consisting of: RB1, TP53, ATM, RAD50, FBXW7 and PARK2.

105. The method of any of claims 1-104, wherein the subject is human papillomavirus (HPV) positive.

106. The method of any of one claims 1-105, wherein gemcitabine is administered daily.

107. The method of claim 105 or 106, wherein gemcitabine is administered on day 1 and the SRA737 compound is administered on days 2 and 3 of a weekly schedule.

108. The method of claim 106 or 107, wherein gemcitabine and the SRA737 compound are administered over one or more 28 day cycles.

109. The method of claim 108, wherein gemcitabine is administered on days 1, 8, and 15 of the one or more 28 day cycles, and the SRA737 compound is administered on days 2, 3, 9, 10, 16, and 17 of the one or more 28 day cycles.

110. The method of any one of claims 106-109, wherein the second effective amount of gemcitabine is selected from the group consisting of: 50 mg/m²/day, 100 mg/m²/day, 150 mg/m²/day, 200 mg/m²/day, 250 mg/m²/day, and 300 mg/m²/day.

111. The method of any one of claims 106-109, wherein the second effective amount of gemcitabine is 600 mg/m²/day or less.

112. The method of any one of claims 106-109, wherein the second effective amount of gemcitabine is between 50 and 600 mg/m²/day.

113. The method of any one of claims 106-109, wherein the second effective amount of gemcitabine is between 50 and 300 mg/m²/day.

114. The method of any one of claims 106-109, wherein the effective amount of the SRA737 compound is 80 mg/day and the second effective amount of gemcitabine is selected from the group consisting of: 50 mg/m²/day, 100 mg/m²/day, 150 mg/m²/day, 200 mg/m²/day, 250 mg/m²/day, and 300 mg/m²/day.

115. The method of any one of claims 106-109, wherein the effective amount of the SRA737 compound is 150 mg/day and the second effective amount of gemcitabine is selected from the group consisting of: 50 mg/m²/day, 100 mg/m²/day, 150 mg/m²/day, 200 mg/m²/day, 250 mg/m²/day, and 300 mg/m²/day.

116. The method of any one of claims 106-109, wherein the effective amount of the SRA737 compound is 300 mg/day and the second effective amount of gemcitabine is selected from the group consisting of: 50 mg/m²/day, 100 mg/m²/day, 150 mg/m²/day, 200 mg/m²/day, 250 mg/m²/day, and 300 mg/m²/day.

117. The method of any one of claims 106-109, wherein the effective amount of the SRA737 compound is 500 mg/day and the second effective amount of gemcitabine is selected from the group consisting of: 50 mg/m²/day, 100 mg/m²/day, 150 mg/m²/day, 200 mg/m²/day, 250 mg/m²/day, and 300 mg/m²/day.

118. The method of any one of claims 106-109, wherein the effective amount of the SRA737 compound is 600 mg/day and the second effective amount of gemcitabine is selected from the group consisting of: 50 mg/m²/day, 100 mg/m²/day, 150 mg/m²/day, 200 mg/m²/day, 250 mg/m²/day, and 300 mg/m²/day.

119. The method of any one of claims 106-109, wherein the effective amount of the SRA737 compound is 700 mg/day and the second effective amount of gemcitabine is selected from the group consisting of: 50 mg/m²/day, 100 mg/m²/day, 150 mg/m²/day, 200 mg/m²/day, 250 mg/m²/day, and 300 mg/m²/day.

120. The method of any one of claims 106-109, wherein the effective amount of the SRA737 compound is 800 mg/day and the second effective amount of gemcitabine is selected from the group consisting of: 50 mg/m²/day, 100 mg/m²/day, 150 mg/m²/day, 200 mg/m²/day, 250 mg/m²/day, and 300 mg/m²/day.

121. The method of any one of claims 106-109, wherein the effective amount of the SRA737 compound is 900 mg/day and the second effective amount of gemcitabine is selected from the group consisting of: 50 mg/m²/day, 100 mg/m²/day, 150 mg/m²/day, 200 mg/m²/day, 250 mg/m²/day, and 300 mg/m²/day.

122. The method of any one of claims 106-109, wherein the effective amount of the SRA737 compound is 1000 mg/day and the second effective amount of gemcitabine is selected from the group consisting of: 50 mg/m²/day, 100 mg/m²/day, 150 mg/m²/day, 200 mg/m²/day, 250 mg/m²/day, and 300 mg/m²/day.

123. The method of any of claims 106-122, wherein the cancer is urothelial carcinoma.

124. The method of claim 123, wherein the urothelial carcinoma is selected from the group consisting of: (a) unresectable urothelial carcinomas of the bladder, upper urinary tract, or urethra, and (b) metastatic urothelial carcinomas of the bladder, upper urinary tract, or urethra.

125. The method of any one of claims 106-122, wherein the cancer is HGSOC.

126. The method of claim 125, wherein a tumor associated with the HGSOC is identified as having somatic or germline BRCA1 and BRCA2 wild-type status

127. The method of any one of claims 106-122, wherein the cancer is small cell lung cancer.

128. The method of any one of claims 106-122, wherein the cancer is soft tissue sarcoma.

129. The method of claim 128, wherein the soft tissue sarcoma is selected from the group consisting of: undifferentiated pleiomorphic sarcoma, malignant fibrous histiocytoma (MFH)/high-grade spindle cell sarcoma, pleomorphic liposarcomas, leiomyosarcoma, and dedifferentiated liposarcoma.

130. The method of any one of claims 106-122, wherein the cancer is cervical or anogenital cancer.

131. The method of claim 130, wherein the cervical or anogenital cancer is selected from the group consisting of: advanced/metastatic squamous cell carcinoma of the anus, penis, vagina, and vulva.

132. The method of any one of claims 1-131, wherein the growth inhibition of the tumor associated with the cancer is a minimum growth inhibition of at least 10%, at least 20%, at least 30%, at least 40%, at least 50%, at least 60%, at least 70%, at least 80%, or at least 90% relative to an untreated tumor.

133. The method of any one of claims 1-132, wherein the method results in a regression of a tumor associated with the cancer relative to a baseline measurement.

134. The method of claim 133, wherein the regression is a 30% regression of the tumor associated with the cancer relative to the baseline measurement.

135. The method of claim 133, wherein the regression is a complete regression of the tumor associated with the cancer relative to the baseline measurement.

136. The method of any one of claims 1-135, wherein the method results in cytotoxicity of a tumor associated with the cancer.

137. The method of any one of claims 1-136, wherein the method results in a partial response, a complete response, or a stable disease in the subject relative to a baseline measurement.

138. The method of any one of claims 1-13636, wherein the method results in a partial response in the subject relative to a baseline measurement.

139. The method of any one of claims 1-136, wherein the method results in a complete response in the subject relative to a baseline measurement.

140. The method of any one of claims 1-136, wherein the method results in a stable disease in the subject relative to a baseline measurement.

141. The method of any one of claims 1-140, wherein the method results in a plasma Cₘᵢₙ of at least 100 ng/ml of the SRA737 compound for at least 24 hours in the subject after administration.

142. The method of any one of claims 1-140, wherein the method results in a plasma Cₘᵢₙ of at least 100 nM of the SRA737 compound for at least 24 hours in the subject after administration.

143. The method of any one of claims 1-142, wherein the method results in a plasma AUC₀₋₂₄ of at least 100 ng·h/mL, at least 300 ng·h/mL, at least 600 ng·h/mL, at least 800 ng·h/mL, at least 1000 ng·h/mL, at least 1600 ng·h/mL, at least 2300 ng·h/mL, at least 2500 ng·h/mL, at least 3000 ng·h/mL, at least 3500 ng·h/mL, at least 8000 ng·h/mL, at least 12000 ng·h/mL, at least 15000 ng·h/mL, at least 18000 ng·h/mL, at least 20000 ng·h/mL, at least 25000 ng·h/mL, or at least 29000 ng·h/mL of the SRA737 compound in the subject after administration.

144. The method of any one of claims 1-142, wherein the method results in a plasma AUC₀₋₁₂ of at least 400 ng·h/mL, at least 500 ng·h/mL, at least 600 ng·h/mL, at least 1600 ng·h/mL, at least 2600 ng·h/mL, at least 4500 ng·h/mL, at least 5000 ng·h/mL, at least 8000 ng·h/mL, at least 8000 ng·h/mL, at least 1000 ng·h/mL of the SRA737 compound in the subject after administration.

145. The method of any one of claims 1-142, wherein the method results in a plasma Cₘₐₓ of at least 500 ng/mL, at least 600 ng/mL, at least 800 ng/mL, at least 100 ng/mL, at least 150 ng/mL, at least 175 ng/mL, at least 350 ng/mL, at least 990 ng/mL, at least 1980 ng/mL, at least 2000 ng/mL, or at least 3228 ng/mL of the SRA737 compound in the subject after administration.

146. The method of any one of claims 1-142, wherein the method results in a plasma Cₘₐₓ of less than 500 ng/mL, less than 600 ng/mL, less than 800 ng/mL, less than 100 ng/mL, less than 150 ng/mL, less than 175 ng/mL, less than 350 ng/mL, less than 990 ng/mL, less than 1980 ng/mL, less than 2000 ng/mL, or less than 3228 ng/mL of the SRA737 compound in the subject after administration.

147. The method of any one of claims 1-146, wherein the method results in a plasma Cₘₐₓ between 500 and 3200 ng/mL of the SRA737 compound in the subject after administration.

148. The method of any one of claims 1-146, wherein the method results in a plasma Cₘₐₓ between 500 and 2400 ng/mL of the SRA737 compound in the subject after administration.

149. The method of any one of claims 1-146, wherein the method results in a plasma Cₘₐₓ between 500 and 650 ng/mL of the SRA737 compound in the subject after administration.

150. The method of any one of claims 1-146, wherein the method results in a plasma Cₘₐₓ between 500 and 550 ng/mL of the SRA737 compound in the subject after administration.

151. The method of any one of claims 1-146, wherein the method results in a plasma Cₘₐₓ between 500 and 5500 ng/mL of the SRA737 compound in the subject after administration.

152. The method of any one of claims 1-146, wherein the method results in a plasma Cₘₐₓ between 500 and 4000 ng/mL of the SRA737 compound in the subject after administration.

153. The method of any one of claims 1-152, wherein the subject has fasted prior to administering the effective amount of the SRA737 compound.

154. The method of claim 153, wherein the subject has fasted 30 minutes or more, 1 hour or more, 2 hours or more, 3 hours or more, or 4 hours or more prior to administering the effective amount of the SRA737 compound.

155. The method of claim 154, wherein the subject has fasted 2 hours or more prior to administering the effective amount of the SRA737 compound.

156. The method of any one of claims 1-155, further comprising the subject fasting following administering the effective amount of the SRA737 compound.

157. The method of claim 156, wherein the subject fasts 30 minutes or more, 1 hour or more, 2 hours or more, 3 hours or more, or 4 hours or more following administering the effective amount of the SRA737 compound.

158. The method of claim 156, wherein the subject fasts 1 hour or more following administering the effective amount of the SRA737 compound.

159. A method of activating an innate immune signaling pathway in a cell, the method comprising:
contacting the cell with an effective amount of at least one of, or all of, or any combination of gemcitabine, SRA737, and an immune checkpoint inhibitor,
wherein the activation of the innate immune signaling pathway synergizes with the immune checkpoint inhibitor to reduce growth of a tumor.

160. The method of claim 159, wherein the innate immune signaling pathway is Stimulator of Interferon Genes (STING) pathway or the interferon signaling pathway.

161. A method of inhibiting a Chk1 activity in a cell, the method comprising contacting the cell with an effective amount of at least one of, or all of, or any combination thereof gemcitabine, SRA737, and an immune checkpoint inhibitor, wherein inhibition of the Chk1 activity synergizes with the immune checkpoint inhibitor to reduce growth of a tumor.

162. A method of modulating expression of immune signaling cell surface markers within a cell, the method comprising contacting the cell with a first effective amount of SRA737 and a first effective amount of an immune checkpoint inhibitor, wherein the modulation of the expression of the immune signaling cell surface markers synergizes with the immune checkpoint inhibitor to reduce growth of a tumor.

163. A composition comprising gemcitabine, SRA737, and an immune checkpoint inhibitor.

164. The composition of claim 163, wherein the immune checkpoint inhibitor is selected from the group consisting of an anti-CTLA-4 antibody, an anti-PD-L1 antibody, an anti-PD1 antibody, and an IDO1 inhibitor.

165. A pharmaceutical composition comprising the combination of claim 163 or claim 164, and at least one pharmaceutically acceptable carrier or excipient.

166. A kit comprising the combination of claim 163 or claim 164 or the pharmaceutical composition of claim 33 and instructions for use.

167. The method of any one of claims 1-166, wherein the subject is human.
